(19) United States
(12) Patent Application Publication
(10) Pub. No.: US 2024/0318145 A1
(43) Pub. Date: Sep. 26, 2024

(54) STRUCTURALLY COMPLETE ORGANOIDS

(71) Applicant: Children's Hospital Medical Center, Cincinnati, OH (US)

(72) Inventors: James Macormack Wells, Cincinnati, OH (US); Alexandra Kay Eicher, Cincinnati, OH (US)

(21) Appl. No.: 18/573,954

(22) PCT Filed: Jun. 30, 2022
(86) PCT No.: PCT/US2022/035689

§ 371 Date: Dec. 22, 2023

Related U.S. Application Data
(60) Provisional application No. 63/221,916, filed on Jul. 14, 2021.
(60) Provisional application No. 63/202,998, filed on Jul. 2, 2021.

(51) Int. Cl. C12N 5/071

(52) U.S. Cl. CPC C12N 5/0697; C12N 2500/38; C12N 2501/11; C12N 2501/115; C12N 2501/119; C12N 2501/155; C12N 2501/415; C12N 2501/727; C12N 2503/00; C12N 2506/45; C12N 2513/00

(57) ABSTRACT

Disclosed herein are compositions of gastrointestinal organoids comprising cells originating from all three primary germ layers and methods of making and use thereof. These gastrointestinal organoids exhibit complex cellular organization and functions resembling naturally occurring organ tissue, and serve as excellent three dimensional models for studying gastrointestinal physiology.

### CROSS-REFERENCE TO RELATED APPLICATIONS

This application claims priority to and benefit of U.S. Provisional Patent Application 63/202,998, filed Jul. 2, 2021, and U.S. Provisional Patent Application 63/221,916, filed Jul. 14, 2021, the contents of each of which are incorporated herein by reference in their entirety for all purposes.

### STATEMENT REGARDING FEDERALLY SPONSORED R&D

This invention was made with government support under U19 AI116491, P01 HD093363, UG3 DK119982, U01 DK103117, P30 DK078392, U18 EB021780, 1F31DK118823-01, and T32-ES007250-29 awarded by the National Institutes of Health. The government has certain rights to the invention.

### FIELD OF THE INVENTION

Aspects of the present disclosure relate generally to organoid compositions comprising structurally complete organization of cellular types derived from all three primary germ layers and methods of making and use thereof.

### BACKGROUND

All organs of the gastrointestinal (GI) tract are assembled from cells derived from the three primary germ layers during embryonic development. These diverse cell types are required for the proper execution of the GI tract's complex functions. For example, key functions of the stomach to chemically and mechanically break down orally ingested nutrients depend on a complex interaction of the epithelium to produce acid and proteases, the smooth muscle to contract and relax, and the enteric nerves to provide input and coordinate both of these processes. These three main components of the stomach develop separately from the three primary germ layers with the endoderm forming the epithelial lining, the mesoderm contributing to the stromal cells and smooth muscle layers, and the ectoderm giving rise to the enteric nervous system (ENS), yet come together to form a complete and complex layered structure that is functional by the time of birth. Then, each germ layer plays essential roles in postnatal function. The gastric ENS, as an intrinsic postganglionic network of excitatory and inhibitory neurons, along with the vagus nerve, coordinates the epithelial release of acid and proteases and the relaxation of smooth muscle needed for gastric emptying.

Three dimension gastrointestinal organoids differentiated from stem cells such as induced pluripotent stem cells promise great utility in many aspects of physiology research, pharmaceutical screening, and personalized medicine (as organoids can be produced from patient-derived induced pluripotent stem cells). Previous methods of producing gastrointestinal organoids involve the step wise differentiation of stem cells into definitive endoderm and gut endoderm lineages. The resultant organoids exhibit an organized epithelial and mesenchymal layer. Notably, however, these organoids lack a robust enteric nervous system, which arise from the ectoderm. Furthermore, while mesenchyme is present, variability is observed in different gastrointestinal organoid types. There is a present need for improved methods for producing organoids that more closely resemble that of naturally occurring tissue.

### SUMMARY

Disclosed herein are methods for preparing a gastrointestinal organoid from the three primary germ layers. In some embodiments, the methods comprise contacting gut endoderm spheroids with splanchnic mesoderm cells (SM) and enteric neural crest cells (ENCCs) to form a cell mixture; and culturing the cell mixture under conditions sufficient to differentiate the cell mixture into a gastrointestinal organoid comprising epithelium, mesenchyme, and a functional enteric nervous system (ENS).

Also disclosed herein are methods for preparing Brunner's gland-like organoids. In some embodiments, the methods comprise contacting posterior foregut endoderm spheroids with ENCCs; and culturing the posterior foregut endoderm spheroids and ENCCs under conditions sufficient to differentiate the cell mixture into the Brunner's gland-like organoids; where the presence of ENCCs promotes a more posterior fate for the posterior foregut endoderm spheroids; and where the Brunner's gland-like organoids comprise a glandular epithelium.

Also disclosed herein are the gastrointestinal organoids and Brunner's gland-like organoids produced according to any one of the methods disclosed herein.

Also disclosed herein are methods of screening, such as with a compound of interest, using any one of the gastrointestinal organoids or Brunner's gland-like organoids disclosed herein.

Exemplary embodiments of the present disclosure are provided in the following numbered embodiments:
1. A method for preparing a gastrointestinal organoid from the three primary germ layers, comprising:
contacting gut endoderm spheroids with splanchnic mesoderm cells (SM) and enteric neural crest cells (ENCCs) to form a cell mixture; and
culturing the cell mixture under conditions sufficient to differentiate the cell mixture into a gastrointestinal organoid comprising epithelium, mesenchyme, and a functional enteric nervous system (ENS).
2. The method of embodiment 1, wherein one or more of the gut endoderm spheroids, the SM, or the ENCCs have been derived from pluripotent stem cells.
3. The method of embodiment 1 or 2, wherein the gut endoderm spheroids have been derived from definitive endoderm cells.
4. The method of embodiment 3, wherein the definitive endoderm cells have been derived from pluripotent stem cells.
5. The method of any one of embodiments 1-4, wherein the gut endoderm spheroids are spontaneously formed gut endoderm spheroids that develop during differentiation of definitive endoderm cells into gut endoderm.
6. The method of any one of embodiments 1-5, wherein the SM and ENCCs are not contacted with a suspension of single gut endoderm cells or aggregated gut endoderm spheroids that are produced by aggregating a suspension of single gut endoderm cells.
7. The method of any one of embodiments 1-6, wherein the gut endoderm spheroids and ENCCs are not contacted with cardiac mesenchyme, septum transversum, or gastric/esophageal mesenchyme cells.
8. The method of any one of embodiments 1-7, wherein the SM have been derived from pluripotent stem cells according to a method comprising:
a) contacting the pluripotent stem cells with a TGF-b pathway activator, a Wnt pathway activator, an FGF pathway activator, a BMP pathway activator, and a PI3K pathway inhibitor for a first period to differentiate the pluripotent stem cells to middle primitive streak cells;
b) contacting the middle primitive streak cells with a TGF-b pathway inhibitor, a Wnt pathway inhibitor, and a BMP pathway activator for a second period to differentiate the middle primitive streak cells to lateral plate mesoderm cells; and
c) contacting the lateral plate mesoderm cells with a TGF-b pathway inhibitor, Wnt pathway inhibitor, an FGF pathway activator, a BMP pathway activator, and retinoic acid for a third period to differentiate the lateral plate mesoderm cells to SM.
9. The method of embodiment 8, wherein the first period is 1, 2, or 3 days, preferably 1 day, the second period is 1, 2, or 3 days, preferably 1 day, and the third period is 1, 2, 3, 4, or 5 days, preferably 2 days.
10. The method of any one of embodiments 1-9, wherein the ENCCs have been derived from pluripotent stem cells according to a method comprising:
a) contacting the pluripotent stem cells with an FGF pathway activator and an EGF pathway activator, preferably EGF, for a first period and the FGF pathway activator, the EGF pathway activator, and retinoic acid for a second period to differentiate the pluripotent stem cells to neurospheres comprising the ENCCs;
b) culturing the neurospheres on an extracellular matrix, preferably fibronectin, under conditions to allow the ENCCs to migrate from the neurospheres as single cells; and
c) collecting the ENCCs that have migrated from the neurospheres as the single cells, thereby producing the ENCCs.
11. The method of embodiment 10, wherein the first period is 3, 4, 5, 6, 7, or 8 days, preferably 5 days, and the second period is 1, 2, 3, or 4 days, preferably 1 day.
12. The method of any one of embodiments 1-11, wherein the gut endoderm spheroids are contacted with the SM at a ratio of about 250, about 500, about 1000, about 1500, about 2000, about 2500, about 3000, about 3500, about 4000, about 4500, or about 5000 SM per gut endoderm spheroid, or any ratio within a range defined by any two of the aforementioned ratios of SM to gut endoderm spheroid.
13. The method of any one of embodiments 1-12, wherein the gut endoderm spheroids are contacted with the SM at a ratio of about 1 to 1, 1.5 to 1, 2 to 1, 2.5 to 1, or 3 to 1 SM to the total number of gut endoderm cells in the gut endoderm spheroid, or any ratio within a range defined by any two of the aforementioned ratios of SM to the total number of gut endoderm cells in the gut endoderm spheroid.
14. The method of any one of embodiments 1-13, wherein the gut endoderm spheroids are contacted with the ENCCs at a ratio of about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 ENCCs per gut endoderm spheroid, or any ratio within a range defined by any two of the aforementioned ratios of ENCCs to gut endoderm spheroid.
15. The method of any one of embodiments 1-14, wherein the gut endoderm spheroids are contacted with the ENCCs at a ratio of about 1 to 1, 1 to 1.25, 1 to 1.5, 1 to 2, 1 to 2.5, or 1 to 3 ENCCs to the total number of gut endoderm cells in the gut endoderm spheroid, or any ratio within a range defined by any two of the aforementioned ratios of ENCCs to the total number of gut endoderm cells in the gut endoderm spheroid.
16. The method of any one of embodiments 1-15, wherein the SM or ENCCs, or both, are in suspension of single cells.
17. The method of any one of embodiments 1-16, wherein the gut endoderm spheroids are contacted with the SM and the ENCCs by low speed centrifugation.
18. The method of any one of embodiments 1-17, wherein the cell mixture is cultured in an extracellular matrix or a derivative or mimic thereof, preferably Matrigel.
19. The method of any one of embodiments 1-18, wherein the gut endoderm spheroids are foregut endoderm spheroids.
20. The method of embodiment 19, wherein the foregut endoderm spheroids are posterior foregut endoderm spheroids, and the gastrointestinal organoid is a gastric organoid.
21. The method of embodiment 20, wherein the posterior foregut endoderm spheroids have been derived from definitive endoderm cells according to a method comprising contacting the definitive endoderm cells with an FGF pathway activator, a BMP pathway inhibitor, and a Wnt pathway activator for a first period and the FGF pathway activator, the BMP pathway activator, the Wnt pathway activator, and retinoic acid for a second period, thereby differentiating the definitive endoderm cells into the posterior foregut endoderm spheroids.
22. The method of embodiment 21, wherein the first period is 1, 2, 3, 4, or 5 days, preferably 3 days, and the second period is 1, 2, or 3 days, preferably 1 day.
23. The method of any one of embodiments 20-22, wherein the gastric organoid is an antral gastric organoid and the conditions sufficient to differentiate the cell mixture to the antral gastric organoid comprises contacting the cell mixture with a BMP pathway inhibitor, an EGF pathway activator, and retinoic acid for a third period and the EGF pathway activator for a fourth period.
24. The method of embodiment 23, wherein the third period is 1, 2, 3, 4, or 5 days, preferably 3 days and the fourth period is 1-16 days.
25. The method of embodiment 23 or 24, wherein the antral gastric organoid comprises PDX1 expression, surface mucous cells expressing MUC5AC, gland mucous cells expressing MUC6, or endocrine cells expressing ghrelin, serotonin, histamine, and gastrin, or any combination thereof.
26. The method of any one of embodiments 23-25, wherein the antral gastric organoid comprises a neural plexus comprising choline acetyltransferase+(CHAT+) and dopaminergic (TH+) neurons in close proximity to the epithelium and/or endocrine cells.
27. The method of any one of embodiments 20-22, wherein the gastric organoid is a fundic gastric organoid and the conditions sufficient to differentiate the cell mixture to the fundic gastric organoid comprises contacting the cell mixture with a BMP pathway inhibitor, a Wnt pathway activator, an EGF pathway activator, and retinoic acid for a third period, and the Wnt pathway activator and the EGF pathway activator for a fourth period.
28. The method of embodiment 27, wherein the third period is 1, 2, 3, 4, or 5 days, preferably 3 days, and the fourth period is 1-16 days.
29. The method of embodiment 27 or 28, wherein the fundic gastric organoid is further contacted with a BMP pathway activator and a MEK pathway inhibitor to induce parietal cell differentiation.
30. The method of any one of embodiments 27-29, wherein the fundic gastric organoid comprises ATP4B+GIF+ parietal cells, PGA3 expression, and lacks PDX1 and gastrin.
31. The method of any one of embodiments 20-30, wherein the gastric organoid comprises about 50% or at least 50% mesenchyme.
32. The method of any one of embodiments 20-31, wherein the mesenchyme of the gastric organoid is capable of differentiating into αSMA+ smooth muscle cell.
33. The method of any one of embodiments 20-32, wherein the gastric organoid comprises the gastric epithelial marker CLDN18 and lacks the intestinal epithelial marker CDH17.
34. The method of any one of embodiments 20-33, wherein the gastric organoid exhibits spontaneous contractile oscillations.
35. The method of embodiment 19, wherein the foregut endoderm spheroids are anterior foregut endoderm spheroids, and the gastrointestinal organoid is an esophageal organoid.
36. The method of embodiment 35, wherein the anterior foregut endoderm spheroids have been derived from definitive endoderm cells according to a method comprising contacting the definitive endoderm cells with an FGF pathway activator and a BMP pathway inhibitor for a first period, thereby differentiating the definitive endoderm cells into the anterior foregut endoderm spheroids.
37. The method of embodiment 36, wherein the first period is 1, 2, 3, 4, or 5 days, preferably 3 days.
38. The method of any one of embodiments 35-37, wherein the conditions sufficient to differentiate the cell mixture to the esophageal organoid comprises contacting the cell mixture with an FGF pathway activator, a BMP pathway inhibitor, and an EGF pathway activator for a second period, and the EGF pathway activator for a third period.
39. The method of embodiment 38, wherein the second period is 1, 2, 3, 4, 5, 6, 7, 8, 9, 10, 11, 12, 13, or 14 days, preferably 7 days, and the third period is 3-58 days.
40. The method of any one of embodiments 35-39, wherein the esophageal organoid comprises about 25% or at least 25% mesenchymal cells.
41. The method of any one of embodiments 35-40, wherein the esophageal organoid comprises about 4% or at least 4% mesenchymal cells expressing FOXF1.
42. The method of any one of embodiments 35-41, wherein the esophageal organoid comprises a TUJ1+ neuronal plexus associated within a FOXF1+ mesenchymal layer.
43. The method of any one of embodiments 1-42, wherein one or more of the gut endoderm spheroids, SM, or ENCCs comprise a detectable marker.
44. The method of embodiment 43, wherein the detectable marker is a fluorescent protein or a luminescent protein.
45. The method of any one of embodiments 1-44, further comprising transplanting the gastrointestinal organoid into a mammal, such as a mouse, such as an immunocompromised mouse.
46. The method of embodiment 45, wherein the gastrointestinal organoid is transplanted to the kidney capsule of the mammal.
47. The method of embodiment 45 or 46, wherein the transplanted gastrointestinal organoid grows about 50×, 150×, 200×, 250×, 300×, 400×, 500×, 600×, 700×, 800×, 900×, 1000×, 1100×, 1200×, 1300×, 1400×, or 1500×, or at least 50×, 150×, 200×, 250×, 300×, 400×, 500×, 600×, 700×, 800×, 900×, 1000×, 1100×, 1200×, 1300×, 1400×, or 1500× in volume following transplantation and/or comprises αSMA+ smooth muscle cells, enteric neurons and epithelium.
48. The method of any one of embodiments 1-47, wherein one or more of the gut endoderm spheroids, SM, or ENCCs comprise one or more altered genes.
49. The method of embodiment 48, wherein the one or more altered genes comprise a gene that is involved in a gastrointestinal disease.
50. The method of embodiment 49, wherein the alteration of the gene that is involved in the gastrointestinal disease induces the gastrointestinal organoid to exhibit the gastrointestinal disease or abrogates the gastrointestinal disease in the gastrointestinal organoid.
51. A gastrointestinal organoid produced by the method of any one of embodiments 1-50.
52. The gastrointestinal organoid of embodiment 51, wherein the gastrointestinal organoid comprises a muscularis mucosa, submucosa, and muscularis externa.
53. The gastrointestinal organoid of embodiment 52, wherein the gastrointestinal organoid comprises plexi of enteric neurons within the submucosa and muscularis externa.
54. A method of preparing Brunner's gland-like organoids, comprising:
contacting posterior foregut endoderm spheroids with ENCCs; and
culturing the posterior foregut endoderm spheroids and ENCCs under conditions sufficient to differentiate the cell mixture into the Brunner's gland-like organoids;
wherein the presence of ENCCs promotes a more posterior fate for the posterior foregut endoderm spheroids; and
wherein the Brunner's gland-like organoids comprise a glandular epithelium.
55. The method of embodiment 54, wherein the posterior foregut endoderm spheroids are not contacted with SM.
56. The method of embodiment 54 or 55, wherein the posterior foregut endoderm spheroids and/or ENCCs have been derived from pluripotent stem cells.
57. The method of any one of embodiments 54-56, wherein the posterior foregut endoderm spheroids have been derived from definitive endoderm cells.
58. The method of embodiment 57, wherein the definitive endoderm cells have been derived from pluripotent stem cells.
59. The method of any one of embodiments 54-58, wherein the posterior foregut endoderm spheroids are spontaneously formed posterior foregut endoderm spheroids that develop during differentiation of definitive endoderm cells into gut endoderm.
60. The method of any one of embodiments 54-59, wherein the ENCCs are not contacted with a suspension of single posterior foregut endoderm cells or aggregated posterior foregut endoderm spheroids that are produced by aggregating a suspension of single posterior foregut endoderm cells.
61. The method of any one of embodiments 54-60, wherein the posterior foregut endoderm spheroids and ENCCs are not contacted with cardiac mesenchyme, septum transversum, or gastric/esophageal mesenchyme cells.
62. The method of any one of embodiments 54-61, wherein the posterior foregut endoderm spheroids have been derived from definitive endoderm cells according to a method comprising contacting the definitive endoderm cells with an FGF pathway activator, a BMP pathway inhibitor, and a Wnt pathway activator for a first period and an FGF pathway activator, a BMP pathway inhibitor, a Wnt pathway activator, and retinoic acid for a second period, thereby differentiating the definitive endoderm cells into the posterior foregut endoderm spheroids.
63. The method of embodiment 62, wherein the first period is 1, 2, 3, 4, or 5 days, preferably 3 days, and the second period is 1, 2, or 3 days, preferably 1 day.
64. The method of any one of embodiments 54-63, wherein the ENCCs have been derived from pluripotent stem cells according to a method comprising:
a) contacting the pluripotent stem cells with an FGF pathway activator and an EGF pathway activator for a third period and with the FGF pathway activator, the EGF pathway activator, and retinoic acid for a fourth period to differentiate the pluripotent stem cells to neurospheres comprising the ENCCs;
b) culturing the neurospheres on an extracellular matrix, preferably fibronectin, under conditions to allow the ENCCs to migrate from the neurospheres as a single cells; and
c) collecting the ENCCs that have migrated from the neurospheres as the single cells, thereby producing the ENCCs.
65. The method of embodiment 64, wherein the third period is 1, 2, 3, 4, or 5 days, preferably 3 days, and the fourth period is 1, 2, 3, or 4 days, preferably 2 days.
66. The method of any one of embodiments 54-65, wherein the posterior foregut endoderm spheroids are contacted with the ENCCs at a ratio of about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 ENCCs per foregut endoderm spheroid, or any ratio within a range defined by any two of the aforementioned ratios of ENCCs to posterior foregut endoderm spheroid.
67. The method of any one of embodiments 54-66, wherein the posterior foregut endoderm spheroids are contacted with the ENCCs by low speed centrifugation.
68. The method of any one of embodiments 54-67, wherein the cell mixture is cultured in an extracellular matrix or a derivative or mimic thereof, preferably Matrigel.
69. The method of any one of embodiments 54-68, wherein the conditions sufficient to differentiate the cell mixture to the Brunner's gland-like organoid comprises contacting the cell mixture with a BMP pathway inhibitor, an EGF pathway activator, and retinoic acid, for a fifth period and, optionally, an EGF pathway activator for a sixth period.
70. The method of embodiment 69, wherein the fifth period is 1, 2, 3, 4, or 5 days, preferably 3 days and the sixth period is 1-16 days.
71. The method of any one of embodiments 54-70, wherein the glandular epithelium of the Brunner's gland-like organoid:
a) expresses PDX1, MUC6, and GLP-1R;
b) lacks expression of CLDN18, CDH17, SOX2, MUC2, and MUC5AC;
c) expresses lower levels of CDX2 relative to duodenal epithelium; or
d) secretes serotonin, ghrelin, histamine, and somatostatin;
or any combination thereof.
72. The method of any one of embodiments 54-71, wherein the posterior foregut endoderm spheroids and/or ENCCs comprise one or more altered genes.
73. The method of embodiment 72, wherein the one or more altered genes comprise a gene that is involved in a gastrointestinal disease.
74. The method of embodiment 73, wherein the alteration of the gene that is involved in the gastrointestinal disease induces the gastrointestinal organoid to exhibit the gastrointestinal disease or abrogates the gastrointestinal disease in the gastrointestinal organoid.
75. A Brunner's gland-like organoid produced by the method of any one of embodiments 54-74.
76. A method of screening, comprising contacting the gastrointestinal organoid of any one of embodiments 51-53 or the Brunner's gland-like organoid of embodiment 75 with a compound of interest and assessing a change in phenotype in the gastrointestinal organoid or the Brunner's gland-like organoid.
77. The method of embodiment 76, wherein the gastrointestinal organoid or the Brunner's gland-like organoid is derived from stem cells obtained from a subject.
78. The method of embodiment 77, wherein the subject comprises a disease and the change in phenotype is associated with an improvement of the disease.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A-F depict embodiments of incorporation of hPSC-derived splanchnic mesenchyme into human antral gastric organoids (hAGOs). FIG. 1A depicts a schematic depicting the method of deriving and incorporating GFP+ splanchnic mesenchyme (SM) into hAGOs. SM was derived from an hPSC line that constitutively expresses GFP. FIG. 1B depicts representative immunostaining of day 4 splanchnic (left) and cardiac (right) mesenchymal monolayers co-stained with FOXF1 and ISL1. FIG. 1C depicts quantification of FOXF1+(left) and ISL1+(right) cells within day 4 splanchnic and cardiac mesenchymal monolayers (n=3 fields from one differentiation, *p<0.05, Student's t-test). FIG. 1D depicts brightfield images of hAGOs grown for four weeks in vitro with and without recombination with exogenous GFP-labeled SM co-stained with mesenchymal marker FOXF1. Higher magnification images are shown to the right. FIG. 1E depicts quantification of FOXF1+ mesenchymal contribution (n=11-18 sections from at least 3 organoids per condition, *** p<0.001, Student's t-test). FIG. 1F depicts representative images of four week in vitro hAGOs with and without recombined SM stained with smooth muscle marker αSMA and gastric epithelial marker CLDN18.

FIG. 2A-D depict embodiments of three germ layer recombinants forming human gastric tissue with innervated layers of smooth muscle and glandular epithelium. FIG. 2A depicts a schematic depicting the generation of three germ layer recombinants using foregut endoderm, splanchnic mesenchyme (SM) and enteric neural crest cells (ENCCs). FIG. 2B depicts morphological comparison between hAGO transplants with and without SM and ENCCs (top) and representative images of 10 week in vivo hAGOs stained with αSMA mesenchyme (bottom). ENS is labeled with GFP and counterstained with epithelial marker ECAD. FIG. 2C depicts marker analysis of gastric epithelial patterning and cell types that develop in three germ layer transplanted hAGOs. MUC5AC (top left) and MUC6 (top middle) mark surface pit and gland mucous cells, respectively. Endocrine cells were identified with the hormones ghrelin (top right), serotonin (bottom left), histamine (bottom middle), and gastrin (right bottom). GFP labels the recombined ENS and the epithelium is labeled with CLDN18 (top) and ECAD (bottom). FIG. 2D depicts marker analysis of neuronal differentiation in three germ layer transplanted hAGOs. GFP positive ENS is co-stained with choline acetyltransferase (CHAT, left) and tyrosine hydroxylase (TH, right).

FIG. 3A-N depict embodiments of a comparison of engineered antral and fundic organoid tissue with the human stomach. Histological and immunofluorescence analysis of tissue from 38 week (FIG. 3A) and adult human (FIG. 3B) stomach taken from the antral/fundic boundary, three germ layer transplanted hAGO (FIG. 3C), and hFGO (FIG. 3D). The three germ layers are labeled with neuronal marker TUJ1 or GFP, smooth muscle αSMA, and epithelial marker ECAD. FIG. 3E depicts analysis of gastric epithelial patterning and cell differentiation of three germ layer transplanted hAGOs (top) and hFGOs (bottom). PDX and the hormone gastrin (GAST) (left and middle) are enriched in the hAGOs. Parietal cells (ATP4B, left), endocrine cells expressing the hormone ghrelin (GHRL, middle left), and chief cells expressing fundic-specific pepsinogen A3 (PGA3, middle right) are enriched in the hFGOs. Pepsinogen C (PGC, middle right) is in both types of organoids. MUC5AC is a surface mucous marker and GIF labels parietal cells (right). Epithelium is labeled in ECAD. FIG. 3F depicts representative images of three germ layer hAGO and hFGO transplants stained for ATP4A and ATP4B, which label parietal cells in both types of gastric organoids. FIG. 3G-N depicts quantification of PDX1, ATP4B, gastrin, ghrelin, MUC5AC, PGC, PGA3, and PGC/PGA3 in three germ layer hAGO and hFGO transplants. Significance denoted as *p<0.05, ** p<0.01, and *** p<0.001 determined by Student's t-test or one-way ANOVA with Tukey's Multiple Comparison.

FIG. 4A-F depict embodiments of antral three germ layer organoids having a functional ENS that regulates gastric tissue contractions. FIG. 4A depicts isometric force contractions in tissues isolated from three individual transplanted hAGO +SM +ENS. Contractile activity was triggered using electrical field stimulation (EFS). FIG. 4B depicts neuronal (TUJ1) and interstitial cells of Cajal (ICC) (c-KIT) stained in 13 week in vivo hAGO +SM +ENS graft.

FIG. 4C depicts activation of muscarinic receptors induced contractions in tissues isolated from a transplanted hAGO +SM +ENS. Increasing doses of bethanechol were added to the tissues at times indicated by the arrows. FIG. 4D depicts inhibition of the muscarinic receptor with scopolamine induced muscle relaxation and calculated maximal and minimal tissue tension of tissues from hAGO +SM +ENS. FIG. 4E depicts inhibition of ENS activation with the neurotoxin tetrodotoxin (TTX) abrogating EFS-mediated contractions. Change in area under the curve following a control EFS stimulation is measured for one minute after stimulation, followed by TTX treatment, and a final EFS stimulation in hAGO +SM +ENS. FIG. 4F depicts functionally testing the role of nitrergic and cholinergic neuronal activity in smooth muscle contractions. Change in area under the curve induced by EFS stimulation and following treatment with the nitrergic inhibitor L-NAME and the cholinergic inhibitor Atropine. All data was normalized to tissue mass; n=3 for all from one differentiation.

FIG. 5A-E depict embodiments of ENS cells promoting in vitro growth and patterning of gastric mesenchyme. FIG. 5A depicts a schematic illustrating the method of recombining hAGOs with ENCCs at Day 6 and Day 9 of the hAGO protocol. FIG. 5B depicts representative images of four week in vitro hAGOs with (bottom) and without (top) ENS recombined at Day 6 of the hAGO protocol stained with TUJ1 neurons, FOXF1 mesencyme, and ECAD epithelium. Higher magnification images are shown to the right. FIG. 5C depicts representative images of four week in vitro hAGOs with (bottom) and without (top) ENS recombined on either Day 6 (left) or Day 9 (right) of the hAGO protocol, demonstrating an increase in FOXF1+ mesenchyme. FIG. 5D depicts quantification of FOXF1+ mesenchymal contribution (n=16-24 sections from at least 3 organoids, *p<0.05, *** p<0.01, Student's t-test).

FIG. 5E depicts relative expression of key gastric mesenchymal genes (BARX1, BAPX1, FGF10, ISL1, SIX2) in hAGOs +ENS when compared to hAGOs-ENS (n=4-12 wells, with a minimum of 3 organoids per well, from 5 individual differentiations, *p<0.05, Student's t-test).

FIG. 6A-B depict embodiments of ENCCs promoting hAGO engraftment and epithelial growth. FIG. 6A depicts representative low magnification images of gross organoids of ECAD+ epithelium from transplanted hAGOs with and without ENS following recombination at Day 6 or Day 9. FIG. 6B depicts quantification of organoid engraftment and epithelial growth from transplanted hAGOs with and without ENCCs. 24%(5 out of 21) of hAGO +ENCC recombined at Day 9 had complex glandular epithelium.

FIG. 7A-D depict embodiments of identification of hAGO +ENCC glandular epithelium as Brunner's Glands. FIG. 7A depicts glandular epithelium expressing the pan gastrointestinal markers PDX1 and GATA4 but not expressing gastric epithelial marker CLDN18 or intestinal epithelial marker CDH17 in transplanted hAGOs recombined with ENCCs on Day 9. FIG. 7B-D depict marker analysis of organoid epithelium at different time points following transplantation. FIG. 7B depicts images showing that after 6 weeks of growth in vivo, hAGOs with ENCCs recombined at Day 6 had a simple epithelium expressing the gastric markers SOX2 (inset) and CLDN18 but not the intestinal marker CDX2. The glandular epithelium from Day 9 recombinants did not express these gastric or intestinal markers. FIG. 7C depicts a comparison of antral and duodenal markers to known markers of Brunner's glands and how these align with observed protein expression profile of complex epithelial growths from hAGOs +ENS Day 9 recombined grafts. PDX1, GATA4, CLDN18, CDH17, SOX2, CDX2, MUC6, MUC5AC, GLP-1R markers were determined from previously published data (“+”). CLDN18, SOX2, CDX2, MUC5AC markers of Brunner's glands were determined experimentally on human tissue samples of Brunner's glands (“*”). “ND” represents markers that have not been determined. FIG. 7D depicts images showing that at 11 weeks post-transplant, the glandular epithelium in organoids expressed MUC6 (left) and GLP-1R (middle), similar to human Brunner's glands. The simple epithelium (arrowhead) expressed MUC5AC (right) while the complex glandular epithelium (arrow) did not.

FIG. 8A-C depict embodiments of data showing that splanchnic mesenchymal recombination yielded the most added exogenous mesenchyme while still retaining endogenous mesenchyme. FIG. 8A depicts brightfield images of 4 week in vitro hAGOs recombined with varying concentrations of splanchnic and septum transversum (STM) mesenchyme on day 6 of hAGO protocol. Visual qualitative assessment of 4 week in vitro hAGOs lead to utilizing splanchnic mesenchyme at a ratio of 50,000 cells/well of approximately 20-30 hAGO spheroids. This equates to an approximately 2:1 ratio of splanchnic mesenchymal cells to hAGO epithelial cells. FIG. 8B depicts brightfield images of hAGOs grown for four weeks in vitro with and without recombination with exogenous GFP-labeled gastric-esophageal mesenchyme (GEM) co-stained with mesenchymal marker FOXF1. Higher magnification images are shown to the right. FIG. 8C depicts quantification of various mesenchymal recombination techniques, including day 6 mesenchymal recombination (left) of either GFP+ splanchnic (SM or cardiac (CM) mesenchyme and day 9 mesenchymal recombination (right) of either GFP+ GEM or STM mesenchyme (n=at least 6 sections from at least 3 organoids per condition, ** p<0.01, Student's t-test).

FIG. 9A-H depict embodiments of three germ layer in vitro and in vivo hAGOs and hFGOs containing GFP+ splanchnic mesenchyme and RFP+ENS. FIG. 9A depicts brightfield and fluorescent images of four week in vitro hAGO +GFP SM +RFP ENS, and epithelial ECAD. Higher magnification are shown on the bottom row. FIG. 9B depicts quantification of GFP+ mesenchyme, RFP+ neural, and ECAD+ epithelial populations within four week in vitro hAGOs (n=8 sections from at least 3 organoids). FIG. 9C depicts representative images of gross in vitro and post transplantation hAGOs with and without incorporation of SM and GFP-labeled ENS. GFP neurons formed networks around grafts post transplantation. FIG. 9D depicts quantification of mesenchymal populations within four week in vitro hAGOs. FIG. 9E depicts representative images of the epithelial (1), proximal muscularis mucosa (2), submucosa, and distal muscularis externa (4) layer thickness from hAGOs 12 weeks post transplantation, 38 week old human fetal stomach, and adult stomach. FIG. 9F depicts quantification of the cell layers shown in FIG. 9E. FIG. 9G depicts representative images of gross in vitro and post-transplantation hFGOs with and without incorporation of SM and GFP-labeled ENS. GFP neurons formed networks around grafts post-transplantation. FIG. 9H depicts representative histological and immunofluorescent comparison of hFGOs with and without added SM and GFP-ENS as well as with and without added BMP4 and MEK pathway inhibitor PD03 to stimulate parietal cell differentiation. Neurons are labeled with TUJ1 (middle), smooth muscle with αSMA (middle), antrum identity with PDX1 (right), and parietal cells with ATP4B (right). Epithelium is labeled with ECAD. Fluorescence intensity is the same as transplanted three germ layer hFGOs (FIG. 3D-E).

FIG. 10A-H depict embodiments of constructing three germ layer organoids in vitro applicable to human esophageal organoids (hEOs). FIG. 10A depicts brightfield and GFP-fluorescent images of 1 month in vitro hEOs. GFP cells label exogenous hPSC-derived SM. Immunofluorescent images of representative hEOs depicting GFP+, FOXF1+ mesenchymal, and Vimentin+(VIM) mesenchymal cells. FIG. 10B depicts quantification of different mesenchymal populations within 1 month in vitro hEOs. FOXF1+ expressing cells without GFP mark endogenous mesenchyme, while both GFP+ groups represent exogenous SM (n=16-18 sections from at least 3 organoids per condition, ** p<0.01, *** p<0.001, Student's t-test). FIG. 10C depict brightfield images of 2 month in vitro hEOs+/−ENS showing a visible expansion of additional cells within hEOs incorporated with ENS. FIG. 10D depict immunofluorescent images of 1 month in vitro hEOs depicting TUJ1+ enteric neurons surrounding the KRT5+ and ECAD+ epithelium of hEOs+ENS. Higher magnification images are shown to the right. FIG. 10E depicts relative expression of neuronal-specific genes including tubulin genes, TUJ1 and MAP2, and filament gene Nestin within 1 month hEOs+ENS (n=3, representative of 3 individual experiments, ** p<0.01, *** p<0.001, Student's t-test). FIG. 10F depicts brightfield images of 1 month in vitro hEOs+/−SM+/−ENS showing a visible expansion of additional cells within hEOs incorporated with ENS and immunofluorescent images of 1 month in vitro hEOs depicting TUJ1+ enteric neurons and FOXF1+ mesenchyme surrounding the KRT8+ epithelium of hEOs +SM+ENS. Higher magnification images are shown to the right. FIG. 10G depict brightfield and fluorescent images of 1 month in vitro hEOs +GFP SM +RFP ENS. Higher magnification images are shown on the bottom row. ECAD marks the epithelium. FIG. 10H depicts quantification of GFP mesenchyme, RFP neural, and ECAD+ epithelial populations within 1 month in vitro hEOs (n=12 sections from at least 3 organoids).

FIG. 11A-J depict embodiments of hPSC-derived ENCCs differentiated into neuroglial subtypes when engineered into hAGOs without exogenous mesenchyme. FIG. 11A depicts a schematic depicting a method of deriving and innervating hAGOs. FIG. 11B depicts representative brightfield (left) and GFP fluorescent (right) images of four week in vitro hAGOs with and without GFP+ ENS. FIG. 11C depict wholemount immunofluorescence of four week in vitro hAGO +ENS labeled with TUJ1+ neurons. FIG. 11D depict immunofluorescent images of TUJ1+ neurons (top) and S100b+ glial cells (bottom) co-expressed with GFP labeled ENCCs.

FIG. 11E depicts quantification of the neuroglial composition co-expressing GFP (n=6, *** p<0.001, Student's t-test). FIG. 11F depicts immunofluorescent images of specific neuronal subtypes, including inhibitory neurons (nNOS) and synaptophysin (SYNAP), dopaminergic neurons (TH), and sensory neurons (CALB1) in hAGOs +ENS. Representative images and quantification of TUJ1+ neurons (FIG. 11G-H) and nNOS+ inhibitory neurons (FIG. 11I-J) within four week in vitro hAGOs +ENS (top) and e13.5 WT murine stomach (bottom) (n>2). Epithelium is labeled with ECAD. Right panels are higher magnification insets of left panels.

FIG. 12A-F depict embodiments of ENS cells supporting in vivo growth and survival of hAGOs. FIG. 12A depict a schematic illustrating the method of transplanting hAGOs +ENS. FIG. 12B depicts quantification of epithelial growth from transplanted hAGOs with and without ENS (n=46-48 transplants per condition from 6 individual differentiations). FIG. 12C depict representative brightfield (left) and GFP fluorescent (right) images of transplanted hAGOs with and without GFP+ ENS following in vivo transplantation (n=29). FIG. 12D depicts brightfield (left) and immunofluorescent (right) images of ECAD+ epithelium from in vivo hAGOs with or without ENS cystic grafts. Representative images of differentiated antral epithelial (FIG. 12E) and mesenchymal and neuronal cell types (FIG. 12F) in hAGOs +ENS following in vivo growth. In FIG. 12E, endocrine cells (arrow) are marked with gastrin, ghrelin, somatostatin, and serotonin, and surface mucous cells are marked by MUC5AC. In FIG. 12F, mesenchymal cells are marked with FOXF1+ with smooth muscle marked with αSMA. Lineage-traced hPSC-derived ENCCs are marked by GFP and differentiated inhibitory neurons are marked with nNOS. Sections were counterstained with epithelial marker ECAD and nuclear DAPI.

FIG. 13A-D depict embodiments of transplanted hAGO grafts +ENS containing appropriate neuroglial cell types that are able to efflux calcium. FIG. 13A depict immunofluorescent images of in vivo hAGOs +ENS showing presence of TUJ1+ neural and S100b+ glial cells as well as differentiated neuronal subtypes marked by peripherin and nNOS. ECAD marks the epithelium. FIG. 13B depicts wholemount immunohistochemistry of in vivo hAGO +ENS showing a 3D network formation of TUJ1+ neurons within αSMA+ smooth muscle layers. FIG. 13C depicts brightfield images of the in vivo hAGO +ENS grafts used to obtain live images of GCaMP neuronal firing. FIG. 13D depicts GFP fluorescent static images taken from live-imaged movie depicting firing of two individual neurons, indicated by the arrow and arrowhead.

FIG. 14A-B depict embodiments of defining Brunner's gland epithelium using combinatorial marker expression analysis of human Brunner's glands. FIG. 14A depicts H&E (left) and immunofluorescent (right) images of adult human Brunner's glands labeled with intestinal epithelial marker CDH17. FIG. 14B depicts immunofluorescent comparison of adult human antrum (top) and duodenum and Brunner's glands (bottom). The gastric epithelial cell types are labeled with CLDN18, SOX2, MUC5AC, PGA3, and PGC. Intestinal cell types are labeled with markers CDX2 and MUC2. Endocrine hormone GAST (middle left) was observed in all regions. Only PGC and GAST were consistently observed in Brunner's glands. Epithelium was labeled with ECAD or β-catenin.

FIG. 15A-D depicts embodiments of Brunner's gland-like epithelium, which only develop from hAGOs innervated by ENCCs untreated with Noggin and retinoic acid. FIG. 15A depicts relative expression of BMP ligands (BMP4 and BMP7) at different points of ENCC differentiation. hPSCs and Day 6 neurospheres (NSs) were used to compare to ENCCs. FIG. 15B depict relative expression of BMP ligands with and without NOG and RA treatment. FIG. 15C depicts representative images of organoids with ECAD+ epithelium from transplanted hAGOs +ENS following recombination at either Day 6 or Day 9 of the hAGO protocol. FIG. 15D depicts representative images of organoids with ECAD+ epithelium and human nuclei expression from transplanted hAGOs +ENCC at day 9 of hAGO protocol; higher magnification is shown on the right.

### DETAILED DESCRIPTION

The development of human organoid model systems has provided new avenues for patient-specific clinical care and disease modeling. However, all organoid systems are missing important cell types that, in the embryo, get incorporated into organ tissues during development. Based on the concept of how embryonic organs are assembled, an organoid assembly approach is disclosed herein, starting with cells from the three primary germ layers; enteric neuroglial, mesenchymal, and epithelial precursors, all separately derived from human pluripotent stem cells. From these, human gastrointestinal tissue can be generated, where the tissue comprises differentiated glands surrounded by layers of smooth muscle containing functional enteric neurons that control contractions of the engineered tissue. Gastric organoids produced by this highly tractable system were used to identify essential roles for the enteric nervous system in the growth and regional identity of the gastric epithelium and mesenchyme and for glandular morphogenesis of the antral stomach. This approach of starting with separately-derived germ layer components was applied to building more complex fundic and esophageal tissue, suggesting this as a new paradigm for tissue engineering.

During organ development, the ENS, mesenchyme, and epithelium communicate with each other in a temporally dynamic manner to regulate regional identity, morphogenesis, and differentiation of progenitors into specific cell types. For example, enteric neural crest cells (ENCCs), which are the neural progenitors of the ENS, actively migrate to the foregut tube in response to signals from the surrounding mesenchyme at the same time as this mesenchyme is differentiating into multiple, organized layers of smooth muscle.

Work with chick embryos have identified specific signaling pathways that mediate reciprocal signaling between germ layers. One common reciprocal signaling module involves sonic hedgehog (Shh), which is secreted by the epithelium of several developing organs and regulates expression of bone morphogenetic protein (BMP) in adjacent mesenchyme. BMP activation then mediates secondary responses such as patterning the developing gut tube mesenchyme and inducing epithelial cell fates like the pyloric sphincter at the junctions of developing organs. Epithelial Shh is also known to indirectly regulate ENCC proliferation and differentiation through manipulation of key extracellular matrix proteins within the gut mesenchyme. Additional studies in chick embryos have shown that ENCCs are required for and regulate the growth, pattering, and maturation of developing stomach mesenchyme. Finally, recent work using both chick and mouse embryos have also shown that both epithelial Shh-induced BMP signaling and ENCC-produced BMP antagonists work in spatiotemporal concert to regulate the radial position of the gut's smooth muscle layers.

Congenital disorders arising from improper ENS and smooth muscle development include neuropathies that can impact the function of the proximal GI tract. This can result, for example, in dysregulation of motility and gastroesophageal reflux disease, collectively described as abnormal gastric emptying. Another more common example of a gastric dysfunction that develops postnatally is gastroparesis. This involves an inability of the pyloric sphincter to relax in coordination with gastric contraction, preventing gastric contents from exiting the stomach which causes bloating and nausea. While the cause of this disorder is unknown, recent work using mouse embryos suggest it may be the result of hypoganglionosis or inflammatory degradation of intrinsic nNOS-expressing inhibitory neurons. Surprisingly little is known about gastric ENS development in any model system, and development of a functional human gastric model system could accelerate not only the study of environmental and genetic factors impacting gastric motility, but also the discoveries of new therapies to improve gastrointestinal function.

While animal models have been invaluable to study gastrointestinal development in vivo and disease, there are major structural and functional differences in gastrointestinal organs between different species. For example, rodents have a forestomach that does not exist in humans. The avian stomach contains a proventriculus, which is a proximal glandular compartment, somewhat paralleling the human fundus, and a gizzard, which is a more distal grinding compartment that is vastly different than the human antrum. There are also developmental differences at the molecular level; Hedgehog signaling appears to play opposite roles in the development of GI smooth muscle between chick and mouse embryos. Each of the existing model systems have unique experimental strengths and weaknesses to study how the gastrointestinal tract forms from the three separate germ layers. Chick embryos are easy to manipulate in vitro, but are not a good genetic model. In contrast, mice a strong genetic model, but germ layer specific studies in vivo are technically demanding or impossible.

As disclosed herein, human pluripotent stem cell (hPSC)-derived splanchnic mesenchyme and ENCCs were incorporated into developing human gastrointestinal organoids, such as antral and fundic gastric organoids (hAGOs and hFGOs, respectively), to recapitulate normal gastric development. The resulting three germ layer organoids were composed of epithelial glands surrounded by multiple layers of functionally innervated smooth muscle. This tractability of this approach was used to study germ layer communication during gastrointestinal development. It was found that human ENCCs promote mesenchymal development glandular morphogenesis and that the presence of adequate amount of mesenchyme is needed for maintaining regional identity.

By understanding and applying the key signaling pathways known to regulate the development of different cell types, many protocols have been developed to direct the differentiation of hPSCs into germ layer-specific fates, including endoderm-derived epithelial cells, mesoderm-derived mesenchymal cells, and ectoderm-derived neural cells. Those principles have been extended herein. To our knowledge, this co-culture of three unique cell types that grew together to form three germ layer organoids is the best hPSCs-derived approximation of bona fide human stomach tissues. The concept of assembling organoids from separately-derived germ layer progenitors was also applied to both fundus and esophagus.

Congenital diseases in humans often affects several organs and can be due to impacts on multiple germ layers. These three germ layer organoids represent new model systems to study both the effects of patient-specific mutations on multiple organs and how gene mutations impact individual germ layers, similar to a cell specific Cre approaches in mice. This approach can be used to study the impact of patient mutations on human PSC-derived ENCCs, on human PSC-derived epithelial cell types, and mutations that largely effect mesenchyme. Analyses of the organoids described herein can be used to identify previously unappreciated patient pathologies that could inform improved clinical care. However, to effectively understand congenital disorders, a better understanding of the processes of epithelial-mesenchyme-ENS communication during normal organ development is needed. The tractability of this system seems ideally suited to interrogate such signaling pathways mediating this crosstalk. The data provided herein suggests that ENCCs impact the growth and patterning of endogenous mesenchyme. Moreover, without addition of exogenous splanchnic mesenchyme, ENCCs re-pattern gastric epithelium to a more posterior identity similar to Brunner's glands. However, when ENCCs are added with a robust population of mesenchyme, together these germ layers maintain gastric patterning and promote gastric gland morphogenesis.

The ability to manipulate signaling pathways at will and in a germ layer-specific manner in vitro is a powerful way to dissect the molecular basis of organ development. For example, it is known that the WNT and BMP signaling pathways control the anterior-posterior and dorso-ventral patterning of the developing GI tract in model organisms and in hPSC-derived colonic organoids. In the gastro-duodenal boundary WNT-mediated crosstalk between the epithelium and mesenchyme are essential for establishing and maintaining a molecular boundary between the gastric and duodenal epithelium. Evidence from chick studies show that ENCCs regulate the anterior-posterior patterning of stomach, and the data here show that ENCCs express BMP ligands and can posteriorize human gastric epithelium, and that inhibiting BMP signaling prevents the posteriorizing effects of ENCCs.

One striking feature of posteriorized gastric organoids is their ability to form Brunner's gland-like structures following transplantation and growth in vivo. Little is known about the embryonic development of these glands in any species, nor what markers define them. Brunner's glands normally form in the proximal duodenum close to the pyloric sphincter and lie just below epithelium. Herein, a marker profile of human Brunner's glands is identified: no expression of the gastric markers SOX2, CLDN18, MUC5AC, no expression of the duodenal markers CDH17, low expression of CDX2, and positive expression for PDX1, the gastric mucin MUC6 and the duodenal marker GLP-1R. Gastric organoids that are mispatterned by ENCCs form a glandular epithelium with a marker profile consistent with Brunner's glands. Human PSC-derived Brunner's gland organoids are a new model system to study development of this glandular system and identify the role of ENCCs in patterning the gastro-duodenal region.

The findings herein highlight that the only context in which formation of normal gastric tissue is observed is when foregut spheroids are combined with a carefully controlled amount of SM and ENCCs. Adding SM alone results in organoids with poorly organized smooth muscle and a simple epithelium, and adding ENCCs alone results in mispatterned epithelium and the formation of Brunner's glands. However, recombining robust populations of SM and ENCCs results in well-organized smooth muscle, an organized neuroglial plexus, and the formation of properly patterned gastric glands with chief and parietal cells. Moreover, the neuroglial plexus forms a functional link with the smooth muscle to regulate rhythmic gastric contractions. Therefore, communication between all three germ layers is important for proper assembly and morphogenesis of stomach tissue.

A possible mechanism to explain why gastric gland morphogenesis requires both ENS and mesenchyme comes from studies of intestinal and lung development. In the intestine of mice and chicks, mesenchymal clusters and smooth muscle regulate villus morphogenesis and lung branches. Additional studies in chick showed how BMP signaling from both the epithelium and neural cells regulates the radial position of developing smooth muscle layers. It follows then that innervation of smooth muscle layers in the human gastric antrum may promote development of organized smooth muscle and glandular morphogenesis. The data herein suggest that ENCCs require a robust population of mesenchyme to promote gastric gland morphogenesis. However, addition of ENCCs alone promotes formation of Brunner's gland-like epithelium in some transplants, suggesting that the addition of mesenchyme is important both to maintain gastric identity and/or synergize with the signals coming from the ENCCs. This seems even more plausible when one considers the close proximity and physical connection of enteric nerves with both the smooth muscle and the epithelial cells within stomach glands, both of which are necessary for proper stomach function. In the case of submandibular glands in mouse, signals from the ENS maintains the epithelial progenitor pools and supports branching morphogenesis in vitro.

In summary, three germ layer organoids can be generated that are morphologically, cellularly, and functionally similar to human stomach tissues. Engineered gastric tissue has glands with surface and pit mucous cells, as wells as chief and parietal cells. Oriented layers of smooth muscle that were innervated by functional enteric nerves can be observed. This highly manipulable system can be used to begin to define cellular communications that happen during development of the human stomach and can serve as a powerful model of gastric diseases. Given that this technology is broadly translatable to other organs, it is possible that engineered tissue might be a source of material for reconstruction of congenital disorders and acute injuries of the upper GI tract.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, Mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox 15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof. Other methods of producing induced pluripotent stem cells as conventionally known in the art are also envisioned.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “differentiation” or “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Cell Differentiation

In some embodiments, known methods for producing downstream cell types from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells or induced pluripotent stem cells. Embryonic stem cells can be derived from the embryonic inner cell mass or from the embryonic gonadal ridges. Embryonic stem cells or germ cells can originate from a variety of animal species including, but not limited to, various mammalian species including humans.

In some embodiments, human embryonic stem cells are used to produce the germ layer cell types used herein. In some embodiments, human embryonic germ cells are used to produce definitive endoderm, gut endoderm, gut endoderm spheroids, splanchnic mesoderm, enteric neural crest cells, or any combination thereof. In some embodiments, iPSCs are used to produce definitive endoderm, gut endoderm, gut endoderm spheroids, splanchnic mesoderm, enteric neural crest cells, or any combination thereof. In some embodiments, human iPSCs (hiPSCs) are used to produce definitive endoderm, gut endoderm, gut endoderm spheroids, splanchnic mesoderm, enteric neural crest cells, or any combination thereof.

In some embodiments, the pluripotent stem cells are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the pluripotent stem cells are treated with one or more small molecule compounds, activators, inhibitors, or growth factors at a concentration that is, is about, is at least, is at least about, is not more than, or is not more than about, 10 ng/ml, 20 ng/mL, 50 ng/mL, 75 ng/mL, 100 ng/mL, 120 ng/mL, 150 ng/mL, 200 ng/mL, 500 ng/ml, 1000 ng/mL, 1200 ng/mL, 1500 ng/ml, 2000 ng/ml, 5000 ng/ml, 7000 ng/mL, 10000 ng/mL, or 15000 ng/ml, or any concentration that is within a range defined by any two of the aforementioned concentrations, for example, 10 ng/ml to 15000 ng/ml, 100 ng/ml to 5000 ng/mL, 500 ng/mL to 2000 ng/mL, 10 ng/mL to 2000 ng/mL, or 1000 ng/mL to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the pluripotent stem cells are cultured in growth media that supports the growth of stem cells. In some embodiments, the pluripotent stem cells are cultured in stem cell growth media. In some embodiments, the stem cell growth media is RPMI 1640, DMEM, DMEM/F12, or Advanced DMEM/F12. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, iPSCs are expanded in cell culture. In some embodiments, iPSCs are expanded in Matrigel. In some embodiments, the iPSCs are expanded in cell culture comprising a ROCK inhibitor (e.g. Y-27632).

In some embodiments, proteins, activators, or inhibitors of the FGF, Wnt, BMP, or retinoic acid pathways, or any combination thereof, are used to mimic development in culture to obtain various cell types used herein that are differentiated from pluripotent stem cells. In some embodiments, cellular constituents associated with the FGF, Wnt, BMP, or retinoic acid (RA) signaling pathways, for example, natural inhibitors, antagonists, activators, or agonists of the pathways can be used to result in inhibition or activation of the FGF, Wnt, BMP, or retinoic acid signaling pathways. In some embodiments, siRNA and/or shRNA targeting cellular constituents associated with the FGF, Wnt, BMP, or retinoic acid signaling pathways are used to inhibit or activate these pathways. Furthermore, the methods disclosed herein may also involve the use of an EGF pathway activator acting as a mitogen, which promotes proliferation and growth of desired cell populations. Modulation of the TGF-b and PI3K pathways are also involved in the preparation of mesoderm lineage cells, including lateral plate mesoderm and splanchnic mesoderm cells.

In some embodiments, pluripotent stem cells, definitive endoderm, gut endoderm, foregut endoderm, hindgut endoderm, lateral plate mesoderm, splanchnic mesoderm, enteric neural crest cells, or any differentiated cells thereof, are contacted with a Wnt pathway activator or Wnt pathway inhibitor. In some embodiments, the Wnt pathway activator comprises a Wnt protein. In some embodiments, the Wnt protein comprises a recombinant Wnt protein. In some embodiments, the Wnt pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt pathway activator comprises a GSK3 pathway inhibitor. In some embodiments, the Wnt pathway activator comprises CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway inhibitor comprises C59, PNU 74654, KY-02111, PRI-724, FH-535, DIF-1, or XAV939, or any combination thereof. In some embodiments, the cells are not treated with a Wnt pathway activator or Wnt pathway inhibitor. The Wnt pathway activator or Wnt pathway inhibitor provided herein may be used in combination with any of the other growth factors, pathway activators, or pathway inhibitors provided herein.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic, and FGF2 is also known as basic fibroblast growth factor (bFGF). Members FGF11, FGF12, FGF13, and FGF14, also known as FGF homologous factors 1-4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF 15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects. In some embodiments, it will be understood by one of skill in the art that any of the FGFs can be used in conjunction with a protein from the Wnt signaling pathway. In some embodiments, the FGF used is one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, FGF23.

In some embodiments, pluripotent stem cells, definitive endoderm, gut endoderm, foregut endoderm, hindgut endoderm, lateral plate mesoderm, splanchnic mesoderm, enteric neural crest cells, or any differentiated cells thereof, are contacted with an FGF pathway activator. In some embodiments, the FGF pathway activator comprises an FGF protein. In some embodiments, the FGF protein comprises a recombinant FGF protein. In some embodiments, the FGF pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF pathway activator. The FGF pathway activator provided herein may be used in combination with any of the other growth factors, pathway activators, or pathway inhibitors provided herein.

In some embodiments, pluripotent stem cells, definitive endoderm, gut endoderm, foregut endoderm, hindgut endoderm, lateral plate mesoderm, splanchnic mesoderm, enteric neural crest cells, or any differentiated cells thereof, are contacted with a BMP pathway activator or BMP pathway inhibitor. In some embodiments, the BMP pathway activator comprises a BMP protein. In some embodiments, the BMP protein is a recombinant BMP protein. In some embodiments, the BMP pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP pathway inhibitor comprises Noggin, RepSox, LY364947, LDN193189, SB431542, or any combination thereof. In some embodiments, the cells are not treated with a BMP pathway activator or BMP pathway inhibitor. The BMP pathway activator or BMP pathway inhibitor provided herein may be used in combination with any of the other growth factors, pathway activators, or pathway inhibitors provided herein.

In some embodiments, pluripotent stem cells, definitive endoderm, gut endoderm, foregut endoderm, hindgut endoderm, lateral plate mesoderm, splanchnic mesoderm, enteric neural crest cells, or any differentiated cells thereof, are contacted with a retinoic acid pathway activator. In some embodiments, the retinoic acid pathway activator comprises retinoic acid, all-trans retinoic acid, 9-cis retinoic acid, CD437, EC23, BS 493, TTNPB, or AM580, or any combination thereof. In some embodiments, the cells are not treated with a retinoic acid pathway activator. The retinoic acid pathway activator provided herein may be used in combination with any of the other growth factors, pathway activators, or pathway inhibitors provided herein.

In some embodiments, pluripotent stem cells, definitive endoderm, gut endoderm, foregut endoderm, hindgut endoderm, lateral plate mesoderm, splanchnic mesoderm, enteric neural crest cells, or any differentiated cells thereof, are contacted with an EGF pathway activator. In some embodiments, the EGF pathway activator is EGF. In some embodiments, the cells are not treated with an EGF pathway activator. The EGF pathway activator provided herein may be used in combination with any of the other growth factors, pathway activators, or pathway inhibitors provided herein.

In some embodiments, pluripotent stem cells, definitive endoderm, gut endoderm, foregut endoderm, hindgut endoderm, lateral plate mesoderm, splanchnic mesoderm, enteric neural crest cells, or any differentiated cells thereof, are contacted with a TGF-beta (TGF-b) pathway activator or TGF-beta pathway inhibitor. In some embodiments, the TGF-beta family comprises bone morphogenetic protein (BMP), growth and differentiation factor (GDF), anti-Müllerian hormone, Activin, and Nodal pathways. In some embodiments, the TGF-beta pathway activator comprises TGF-beta 1, TGF-beta 2, TGF-beta 3, Activin A, Activin B, Nodal, a BMP, IDE1, IDE2, or any combination thereof. In some embodiments, the TGF-beta pathway inhibitor comprises A8301, RepSox, LY365947, SB431542, or any combination thereof. In some embodiments, the cells are not treated with a TGF-beta pathway activator or TGF-beta pathway inhibitor. The TGF-beta pathway activator or TGF-beta pathway inhibitor provided herein may be used in combination with any of the other growth factors, pathway activators, or pathway inhibitors provided herein.

In some embodiments, pluripotent stem cells, definitive endoderm, gut endoderm, foregut endoderm, hindgut endoderm, lateral plate mesoderm, splanchnic mesoderm, enteric neural crest cells, or any differentiated cells thereof, are contacted with a PI3K pathway activator or PI3K pathway inhibitor. In some embodiments, the PI3K pathway activator comprises 740 Y-P, or erucic acid, or both. In some embodiments, the PI3K pathway inhibitor comprises wortmannin, LY294002, hibiscone C, PI-103, IC-87114, ZSTK474, AS-605240, PIK-75, PIK-90, PIK-294, PIK-293, AZD6482, PF-04691502, GSK1059615, quercetin, pluripotin, flurbiprofen, GDC-0941, dactolisib, pictilisib, idelalisib, buparlisib, rigosertib, copanlisib, duvelisib, alpelisib, or any combination thereof. In some embodiments, the cells are not treated with a PI3K pathway activator or PI3K pathway inhibitor. The PI3K pathway activator or PI3K pathway inhibitor provided herein may be used in combination with any of the other growth factors, pathway activators, or pathway inhibitors provided herein.

In some embodiments, for any of the small molecule compounds, pathway activators, pathway inhibitors, or growth factors, the cells are contacted for a time that is, is about, is at least, is at least about, is not more than, or is not more than about, 1 hour, 2 hours, 3 hours, 4 hours, 5 hours, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 1 hour to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, for any of the small molecule compounds, pathway activators, pathway inhibitors, or growth factors, the cells (e.g. pluripotent stem cells, definitive endoderm, gut endoderm, foregut endoderm, hindgut endoderm, lateral plate mesoderm, splanchnic mesoderm, enteric neural crest cells, or any differentiated cells thereof) are contacted in culture such that the concentration of any of the small molecule compounds, signaling pathway activators, signaling pathway inhibitors, or growth factors is at a concentration that is, is about, is at least, is at least about, is not more than, or is not more than about, 10 ng/ml, 20 ng/mL, 50 ng/mL, 75 ng/mL, 100 ng/mL, 120 ng/mL, 150 ng/mL, 200 ng/mL, 500 ng/ml, 1000 ng/mL, 1200 ng/ml, 1500 ng/mL, 2000 ng/mL, 5000 ng/mL, 7000 ng/mL, 10000 ng/mL, or 15000 ng/ml, or any concentration that is within a range defined by any two of the aforementioned concentrations, for example, 10 ng/ml to 15000 ng/ml, 100 ng/ml to 5000 ng/mL, 500 ng/ml to 2000 ng/mL, 10 ng/mL to 2000 ng/mL, or 1000 ng/ml to 15000 ng/mL. In some embodiments, for any of the small molecule compounds, pathway activators, pathway inhibitors, or growth factors, the cells (e.g. pluripotent stem cells, definitive endoderm, gut endoderm, foregut endoderm, hindgut endoderm, lateral plate mesoderm, splanchnic mesoderm, enteric neural crest cells, or any differentiated cells thereof) are contacted in culture such that the concentration of any of the small molecule compounds, pathway activators, pathway inhibitors, or growth factors is at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM. In some embodiments, concentration of small molecule compounds, activators, inhibitors, or growth factors is maintained at a constant level throughout the treatment. In some embodiments, concentration of the small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, cells are differentiated via a “one step” process. For example, one or more molecules that can differentiate pluripotent stem cells into DE culture (e.g., Activin A) are combined with additional molecules that can promote directed differentiation of DE culture (e.g., FGF4, Wnt, Noggin, RA) to directly treat pluripotent stem cells.

In some embodiments, stem cells are treated with one or more growth factors to differentiate to definitive endoderm cells. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, Wnt3a or combinations of any of these growth factors. In some embodiments, the stem cells are contacted with Activin A. In some embodiments, the stem cells are contacted with Activin A and BMP4.

In some embodiments, the iPSCs are differentiated into definitive endoderm cells. In the iPSCs are differentiated into definitive endoderm cells by contacting the iPSCs with Activin A, BMP4, or both. In some embodiments, the iPSCs are contacted with a concentration of Activin A that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration of Activin A within a range defined by any two of the aforementioned concentrations, for example, 10 to 200 ng/mL, 10 to 100 ng/ml, 100 to 200 ng/mL, or 50 to 150 ng/mL. In some embodiments, the iPSCs are contacted with a concentration of BMP4 that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, or 200 ng/ml, or any concentration of BMP4 within a range defined by any two of the aforementioned concentrations, for example, 1 to 200 ng/ml, 1 to 100 ng/mL, 25 to 200 ng/mL, 1 to 80 ng/mL, or 25 to 100 ng/mL.

In some embodiments, the PSCs are differentiated into definitive endoderm cells. In some embodiments, the PSCs are differentiated into gut endoderm cells. In some embodiments, the PSCs are differentiated into foregut or hindgut endoderm cells. In some embodiments, the PSCs are differentiated into lateral plate mesoderm cells. In some embodiments, the PSCs are differentiated into splanchnic mesoderm cells. In some embodiments, the PSCs are differentiated into enteric neural crest cells.

In some embodiments, any of the cells disclosed herein may be cryopreserved for later use. The cells can be cryopreserved according to methods generally known in the art.

### Gastrointestinal Organoid Compositions

Generating various types of gastrointestinal organoids from pluripotent stem cells, and precursor thereof, such as definitive endoderm, gut endoderm, foregut endoderm, or hindgut endoderm, are generally known in the art. Exemplary methods may be found in PCT publications WO 2011/140441, WO 2015/183920, WO 2016/061464, WO 2017/192997, WO 2018/106628, WO 2018/200481, WO 2019/074793, WO 2020/160371, WO 2021/030373, and WO 2020/243633, each of which is hereby expressly incorporated by reference in its entirety.

Methods of producing splanchnic mesoderm from pluripotent stem cells can be found in PCT publication WO 2021/041443, which is hereby expressly incorporated by reference in its entirety.

Methods of producing enteric neural crest cells from pluripotent stem cells and aggregating them with gastrointestinal organoids can be found in PCT publication WO 2016/061464, which is hereby expressly incorporated by reference in its entirety.

Herein are methods of preparing gastrointestinal organoids using cells derived from the three primary germ layers by combining these germ layer cells during culture, differentiation, and maturation of the organoid. The gastrointestinal organoids produced according to these methods exhibit properties that closely resemble naturally occurring gastrointestinal tissue, including intact epithelium and mesenchyme with smooth muscle layers and innervation. In some embodiments, the gastrointestinal organoid resembles one or more gastrointestinal tissues, such as esophageal, gastric, intestinal, or colonic tissue. In some embodiments, the gastrointestinal organoid is an esophageal, gastric, intestinal, or colonic organoid.

Disclosed herein are methods of preparing a gastrointestinal organoid from the three primary germ layers. In some embodiments, the methods comprise contacting gut endoderm spheroids with splanchnic mesoderm cells (SM) and enteric neural crest cells (ENCCs) to form a cell mixture and culturing the cell mixture under conditions sufficient to differentiate the cell mixture into a gastrointestinal organoid comprising epithelium, mesenchyme, and a functional enteric nervous system (ENS). In some embodiments, one or more of the gut endoderm spheroids, the SM, or the ENCCs have been derived from pluripotent stem cells. In some embodiments, the gut endoderm spheroids, SM, and the ENCCs have been derived from pluripotent stem cells. In some embodiments, the gut endoderm spheroids have been derived from definitive endoderm cells. In some embodiments, the definitive endoderm cells have been derived from pluripotent stem cells. In some embodiments, the gut endoderm spheroids are spontaneously formed gut endoderm spheroids that develop during differentiation of definitive endoderm cells into gut endoderm. In some embodiments, the SM and ENCCs are not contacted with a suspension of single gut endoderm cells or aggregated gut endoderm spheroids that are produced by aggregating a suspension of single gut endoderm cells. In some embodiments, the gut endoderm spheroids and ENCCs are not contacted with cardiac mesenchyme, septum transversum, or gastric/esophageal mesenchyme cells. In some embodiments, the SM or ENCCs, or both, are in suspension of single cells. In some embodiments, the gut endoderm spheroids are contacted with the SM and the ENCCs by low speed centrifugation. In some embodiments, the cell mixture is cultured in an extracellular matrix or a derivative or mimic thereof, preferably Matrigel. In some embodiments, one or more of the gut endoderm spheroids, SM, or ENCCs comprise a detectable marker. In some embodiments, the detectable marker is a fluorescent protein or a luminescent protein. In some embodiments, one or more of the gut endoderm spheroids, SM, or ENCCs comprise one or more altered genes. In some embodiments, the one or more altered genes comprise a gene that is involved in a gastrointestinal disease. In some embodiments, the alteration of the gene that is involved in the gastrointestinal disease induces the gastrointestinal organoid to exhibit the gastrointestinal disease or abrogates the gastrointestinal disease in the gastrointestinal organoid.

In some embodiments, the gut endoderm spheroids are contacted with the SM at a ratio that is, is about, is at least, is at least about, is not more than, or is not more than about, 250, about 500, about 1000, about 1500, about 2000, about 2500, about 3000, about 3500, about 4000, about 4500, or about 5000 SM per gut endoderm spheroid, or any ratio within a range defined by any two of the aforementioned ratios of SM to gut endoderm spheroid. In some embodiments, the gut endoderm spheroids are contacted with the SM at a ratio that is, is about, is at least, is at least about, is not more than, or is not more than about, 1 to 1, 1.5 to 1, 2 to 1, 2.5 to 1, or 3 to 1 SM to the total number of gut endoderm cells in the gut endoderm spheroid, or any ratio within a range defined by any two of the aforementioned ratios of SM to the total number of gut endoderm cells in the gut endoderm spheroid

In some embodiments, the gut endoderm spheroids are contacted with the ENCCs at a ratio that is, is about, is at least, is at least about, is not more than, or is not more than about, 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 ENCCs per gut endoderm spheroid, or any ratio within a range defined by any two of the aforementioned ratios of ENCCs to gut endoderm spheroid. In some embodiments, the gut endoderm spheroids are contacted with the ENCCs at a ratio that is, is about, is at least, is at least about, is not more than, or is not more than about, 1 to 1, 1 to 1.25, 1 to 1.5, 1 to 2, 1 to 2.5, or 1 to 3 ENCCs to the total number of gut endoderm cells in the gut endoderm spheroid, or any ratio within a range defined by any two of the aforementioned ratios of ENCCs to the total number of gut endoderm cells in the gut endoderm spheroid.

In some embodiments, the gut endoderm spheroids are foregut endoderm spheroids, which can give rise to foregut gastrointestinal lineages. In some embodiments, the gut endoderm spheroids are hindgut endoderm spheroids, which can give rise to hindgut gastrointestinal lineages.

In some embodiments, gastric organoids can be prepared from the methods disclosed herein. In some embodiments, the foregut endoderm spheroids are posterior foregut endoderm spheroids, and the gastrointestinal organoid is a gastric organoid. In some embodiments, the posterior foregut endoderm spheroids have been derived from definitive endoderm cells according to a method comprising contacting the definitive endoderm cells with an FGF pathway activator, a BMP pathway inhibitor, and a Wnt pathway activator for a first period and the FGF pathway activator, the BMP pathway activator, the Wnt pathway activator, and retinoic acid for a second period, thereby differentiating the definitive endoderm cells into the posterior foregut endoderm spheroids. In some embodiments, the first period is 1, 2, 3, 4, or 5 days, preferably 3 days, and the second period is 1, 2, or 3 days, preferably 1 day. In some embodiments, the FGF pathway activator is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF20, FGF21, FGF22, or FGF23, or any combination thereof. In some embodiments, the FGF pathway activator is FGF4. In some embodiments, the BMP pathway inhibitor is Noggin, RepSox, LY364947, LDN193189, SB431542, or any combination thereof. In some embodiments, the BMP pathway inhibitor is Noggin. In some embodiments, the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, a GSK3 pathway inhibitor, CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway activator is CHIR99021. In some embodiments, the gastric organoid comprises about 50% or at least 50% mesenchyme. In some embodiments, the mesenchyme of the gastric organoid is capable of differentiating into αSMA+ smooth muscle cell. In some embodiments, the gastric organoid comprises the gastric epithelial marker CLDN18 and lacks the intestinal epithelial marker CDH17. In some embodiments, the gastric organoid exhibits spontaneous contractile oscillations.

In some embodiments, the gastric organoid is an antral gastric organoid, and the conditions sufficient to differentiate the cell mixture to the antral gastric organoid comprises contacting the cell mixture with a BMP pathway inhibitor, an EGF pathway activator, and retinoic acid for a third period and the EGF pathway activator for a fourth period. In some embodiments, the third period is 1, 2, 3, 4, or 5 days, preferably 3 days and the fourth period is 1-16 days. In some embodiments, the BMP pathway inhibitor is Noggin, RepSox, LY364947, LDN193189, SB431542, or any combination thereof. In some embodiments, the BMP pathway inhibitor is Noggin. In some embodiments, the EGF pathway activator is EGF. In some embodiments, antral gastric organoid comprises PDX1 expression, surface mucous cells expressing MUC5AC, gland mucous cells expressing MUC6, or endocrine cells expressing ghrelin, serotonin, histamine, and gastrin, or any combination thereof. In some embodiments, the antral gastric organoid comprises a neural plexus comprising choline acetyltransferase+(CHAT+) and dopaminergic (TH+) neurons in close proximity to the epithelium and/or endocrine cells.

In some embodiments, the gastric organoid is a fundic gastric organoid, and the conditions sufficient to differentiate the cell mixture to the fundic gastric organoid comprises contacting the cell mixture with a BMP pathway inhibitor, a Wnt pathway activator, an EGF pathway activator, and retinoic acid for a third period, and the Wnt pathway activator and the EGF pathway activator for a fourth period. In some embodiments, the third period is 1, 2, 3, 4, or 5 days, preferably 3 days, and the fourth period is 1-16 days. In some embodiments, the BMP pathway inhibitor is Noggin, RepSox, LY364947, LDN193189, SB431542, or any combination thereof. In some embodiments, the BMP pathway inhibitor is Noggin. In some embodiments, the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, a GSK3 pathway inhibitor, CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway activator is CHIR99021. In some embodiments, the EGF pathway activator is EGF. In some embodiments, the fundic gastric organoid is further contacted with a BMP pathway activator and a MEK pathway inhibitor to induce parietal cell differentiation. In some embodiments, the BMP pathway activator is BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP pathway activator is BMP4. In some embodiments, the MEK pathway inhibitor is PD0325091. In some embodiments, the fundic gastric organoid comprises ATP4B+GIF+ parietal cells, PGA3 expression, and lacks PDX1 and gastrin.

In some embodiments, esophageal organoids can be prepared from the methods disclosed herein. In some embodiments, the foregut endoderm spheroids are anterior foregut endoderm spheroids, and the gastrointestinal organoid is an esophageal organoid. In some embodiments, the anterior foregut endoderm spheroids have been derived from definitive endoderm cells according to a method comprising contacting the definitive endoderm cells with an FGF pathway activator and a BMP pathway inhibitor for a first period, thereby differentiating the definitive endoderm cells into the anterior foregut endoderm spheroids. In some embodiments, the first period is 1, 2, 3, 4, or 5 days, preferably 3 days. In some embodiments, the conditions sufficient to differentiate the cell mixture to the esophageal organoid comprises contacting the cell mixture with an FGF pathway activator, a BMP pathway inhibitor, and an EGF pathway activator for a second period, and the EGF pathway activator for a third period. In some embodiments, the second period is 1, 2, 3, 4, 5, 6, 7, 8, 9, 10, 11, 12, 13, or 14 days, preferably 7 days, and the third period is 3-58 days. In some embodiments, the FGF pathway activator is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF20, FGF21, FGF22, or FGF23, or any combination thereof. In some embodiments, the FGF pathway activator used to differentiate the definitive endoderm to anterior foregut endoderm spheroids is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF20, FGF21, FGF22, or FGF23, or any combination thereof. In some embodiments, the FGFP pathway activator used to differentiate the definitive endoderm to anterior foregut endoderm spheroids is FGF4. In some embodiments, the FGF pathway activator used to differentiate the cell mixture to the esophageal organoid is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF20, FGF21, FGF22, or FGF23, or any combination thereof. In some embodiments, the FGF pathway activator used to differentiate the cell mixture to the esophageal organoid is FGF10. In some embodiments, the BMP pathway inhibitor is Noggin, RepSox, LY364947, LDN193189, SB431542, or any combination thereof. In some embodiments, the BMP pathway inhibitor is Noggin. In some embodiments, the EGF pathway activator is EGF. In some embodiments, the esophageal organoid comprises about 25% or at least 25% mesenchymal cells. In some embodiments, the esophageal organoid comprises about 4% or at least 4% mesenchymal cells expressing FOXF1. In some embodiments, the esophageal organoid comprises a TUJ1+ neuronal plexus associated within a FOXF1+ mesenchymal layer.

In some embodiments, intestinal organoids can be prepared from the methods disclosed herein. In some embodiments, the gut endoderm spheroids are midgut/hindgut (anterior hindgut) endoderm spheroids, and the gastrointestinal organoid is an intestinal organoid (i.e. small intestine). In some embodiments, the hindgut endoderm spheroids have been prepared according to methods generally known in the art.

In some embodiments, colonic organoids can be prepared from the methods disclosed herein. In some embodiments, the gut endoderm spheroids are hindgut (posterior hindgut) endoderm spheroids, and the gastrointestinal organoid is a colonic organoid. In some embodiments, the hindgut endoderm spheroids have been prepared according to methods generally known in the art.

Any of the methods of producing gastrointestinal organoids may further comprise transplanting the gastrointestinal organoid into a mammal, such as a mouse, such as an immunocompromised mouse. In some embodiments, the gastrointestinal organoid is transplanted to the kidney capsule of the mammal. In some embodiments, the transplanted gastrointestinal organoid grows about 50×, 150×, 200×, 250×, 300×, 400×, 500×, 600×, 700×, 800×, 900×, 1000×, 1100×, 1200×, 1300×, 1400×, or 1500× or at least 50×, 150×, 200×, 250×, 300×, 400×, 500×, 600×, 700×, 800×, 900×, 1000×, 1100×, 1200×, 1300×, 1400×, or 1500× in volume following transplantation and/or comprises αSMA+ smooth muscle cells, enteric neurons and epithelium.

Also disclosed herein are the gastrointestinal organoids produced by any one of the methods disclosed herein. In some embodiments, the gastrointestinal organoid comprises a muscularis mucosa, submucosa, and muscularis externa. In some embodiments, the gastrointestinal organoid comprises plexi of enteric neurons within the submucosa and muscularis externa.

Also disclosed herein are methods of preparing Brunner's gland-like organoids. In some embodiments, the methods comprise contacting posterior foregut endoderm spheroids with ENCCs; and culturing the posterior foregut endoderm spheroids and ENCCs under conditions sufficient to differentiate the cell mixture into the Brunner's gland-like organoids; where the presence of ENCCs promotes a more posterior fate for the posterior foregut endoderm spheroids; and where the Brunner's gland-like organoids comprise a glandular epithelium. In some embodiments, the posterior foregut endoderm spheroids are not contacted with SM. In some embodiments, the posterior foregut endoderm spheroids and/or ENCCs have been derived from pluripotent stem cells. In some embodiments, the posterior foregut endoderm spheroids have been derived from definitive endoderm cells. In some embodiments, the definitive endoderm cells have been derived from pluripotent stem cells. In some embodiments, the posterior foregut endoderm spheroids are spontaneously formed posterior foregut endoderm spheroids that develop during differentiation of definitive endoderm cells into gut endoderm. In some embodiments, the ENCCs are not contacted with a suspension of single posterior foregut endoderm cells or aggregated posterior foregut endoderm spheroids that are produced by aggregating a suspension of single posterior foregut endoderm cells. In some embodiments, the posterior foregut endoderm spheroids and ENCCs are not contacted with cardiac mesenchyme, septum transversum, or gastric/esophageal mesenchyme cells. In some embodiments, the posterior foregut endoderm spheroids are contacted with the ENCCs at a ratio of about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 ENCCs per foregut endoderm spheroid, or any ratio within a range defined by any two of the aforementioned ratios of ENCCs to posterior foregut endoderm spheroid. In some embodiments, the posterior foregut endoderm spheroids are contacted with the ENCCs by low speed centrifugation. In some embodiments, the cell mixture is cultured in an extracellular matrix or a derivative or mimic thereof, preferably Matrigel. In some embodiments, the conditions sufficient to differentiate the cell mixture to the Brunner's gland-like organoid comprises contacting the cell mixture with a BMP pathway activator, an EGF pathway activator, and retinoic acid for a fifth period and, optionally, an EGF pathway 0.52-activator for a sixth period. In some embodiments, the fifth period is 1, 2, 3, 4, or 5 days, preferably 3 days and the sixth period is 1-16 days. In some embodiments, the BMP pathway inhibitor is Noggin, RepSox, LY364947, LDN193189, SB431542, or any combination thereof. In some embodiments, the BMP pathway inhibitor is Noggin. In some embodiments, the EGF pathway activator is EGF. In some embodiments, the glandular epithelium of the Brunner's gland-like organoid: a) expresses PDX1, MUC6, and GLP-1R; b) lacks expression of CLDN18, CDH17, SOX2, MUC2, and MUC5AC; c) expresses lower levels of CDX2 relative to duodenal epithelium; or d) secretes serotonin, ghrelin, histamine, and somatostatin; or any combination thereof. In some embodiments, the posterior foregut endoderm spheroids and/or ENCCs comprise one or more altered genes. In some embodiments, the one or more altered genes comprise a gene that is involved in a gastrointestinal disease. In some embodiments, the alteration of the gene that is involved in the gastrointestinal disease induces the gastrointestinal organoid to exhibit the gastrointestinal disease or abrogates the gastrointestinal disease in the gastrointestinal organoid.

In some embodiments, the posterior foregut endoderm spheroids have been derived from definitive endoderm cells according to a method comprising contacting the definitive endoderm cells with an FGF pathway activator, a BMP pathway inhibitor, and a Wnt pathway activator for a first period and an FGF pathway activator, a BMP pathway inhibitor, a Wnt pathway activator, and retinoic acid for a second period, thereby differentiating the definitive endoderm cells into the posterior foregut endoderm spheroids. In some embodiments, the first period is 1, 2, 3, 4, or 5 days, preferably 3 days, and the second period is 1, 2, or 3 days, preferably 1 day. In some embodiments, the FGF pathway activator is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF20, FGF21, FGF22, or FGF23, or any combination thereof. In some embodiments, the FGF pathway activator is FGF4. In some embodiments, the BMP pathway inhibitor is Noggin, RepSox, LY364947, LDN193189, SB431542, or any combination thereof. In some embodiments, the BMP pathway inhibitor is Noggin. In some embodiments, the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, a GSK3 pathway inhibitor, CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway activator is CHIR99021.

Also disclosed herein are the Brunner's gland-like organoids produced by any one of the methods disclosed herein.

As applied to any of the methods disclosed herein, the SM have been derived from pluripotent stem cells according to a method comprising a) contacting the pluripotent stem cells with a TGF-b pathway activator, a Wnt pathway activator, an FGF pathway activator, a BMP pathway activator, and a PI3K pathway inhibitor for a first period to differentiate the pluripotent stem cells to middle primitive streak cells; b) contacting the middle primitive streak cells with a TGF-b pathway inhibitor, a Wnt pathway inhibitor, and a BMP pathway activator for a second period to differentiate the middle primitive streak cells to lateral plate mesoderm cells; and c) contacting the lateral plate mesoderm cells with a TGF-b pathway inhibitor, Wnt pathway inhibitor, an FGF pathway activator, a BMP pathway activator, and retinoic acid for a third period to differentiate the lateral plate mesoderm cells to SM. In some embodiments, the first period is 1, 2, or 3 days, preferably 1 day, the second period is 1, 2, or 3 days, preferably 1 day, and the third period is 1, 2, 3, 4, or 5 days, preferably 2 days. In some embodiments, the TGF-b pathway activator is TGF-beta 1, TGF-beta 2, TGF-beta 3, Activin A, Activin B, Nodal, a BMP, IDE1, IDE2, or any combination thereof. In some embodiments, the TGF-b pathway activator is Activin A. In some embodiments, the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, a GSK3 pathway inhibitor, CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway activator is CHIR99021. In some embodiments, the FGF pathway activator is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF20, FGF21, FGF22, or FGF23, or any combination thereof. In some embodiments, the FGF pathway activator is FGF2. In some embodiments, the BMP pathway activator is BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP pathway activator is BMP4. In some embodiments, the PI3K pathway inhibitor is wortmannin, LY294002, hibiscone C, PI-103, IC-87114, ZSTK474, AS-605240, PIK-75, PIK-90, PIK-294, PIK-293, AZD6482, PF-04691502, GSK1059615, quercetin, pluripotin, flurbiprofen, GDC-0941, dactolisib, pictilisib, idelalisib, buparlisib, rigosertib, copanlisib, duvelisib, alpelisib, or any combination thereof. In some embodiments, the PI3K pathway inhibitor is PIK-90. In some embodiments, the TGF-b pathway inhibitor is A8301, RepSox, LY365947, SB431542, or any combination thereof. In some embodiments, the TGF-b pathway inhibitor is A8301. In some embodiments, the Wnt pathway inhibitor is C59, PNU 74654, KY-02111, PRI-724, FH-535, DIF-1, or XAV939, or any combination thereof. In some embodiments, the Wnt pathway inhibitor is C59.

As applied to any of the methods disclosed herein, the ENCCs have been derived from pluripotent stem cells according to a method comprising a) contacting the pluripotent stem cells with an FGF pathway activator and an EGF pathway activator, preferably EGF, for a first period and with the FGF pathway activator, the EGF pathway activator, and retinoic acid for a second period to differentiate the pluripotent stem cells to neurospheres comprising the ENCCs; b) culturing the neurospheres on an extracellular matrix, preferably fibronectin, under conditions to allow the ENCCs to migrate from the neurospheres as single cells; and c) collecting the ENCCs that have migrated from the neurospheres as the single cells, thereby producing the ENCCs. In some embodiments, the first period is 3, 4, 5, 6, 7, or 8 days, preferably 5 days, and the second period is 1, 2, 3, or 4 days, preferably 1 day. In some embodiments, the FGF pathway activator is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF20, FGF21, FGF22, or FGF23, or any combination thereof. In some embodiments, the FGF pathway activator is FGF2. In some embodiments, the EGF pathway activator is EGF.

Also disclosed herein are methods of screening. In some embodiments, the methods comprise contacting any one of the gastrointestinal organoids or Brunner's gland-like organoids disclosed herein with a compound of interest and assessing a change in phenotype in the gastrointestinal organoid or the Brunner's gland-like organoid. In some embodiments, the gastrointestinal organoid or the Brunner's gland-like organoid is derived from stem cells obtained from a subject. In some embodiments, the subject comprises a disease and the change in phenotype is associated with an improvement of the disease.

Table 1 depicts different cellular recombination parameters tested for producing hAGOs with all three germ layers. Success is defined by production of the final product hAGO exhibiting the structural and functional properties described herein, including the presence of a complex enteric nervous system, innervated smooth muscle, contractile activity, and glandular epithelium. Failure is defined by the production of an organoid composition lacking one or more of the above aspects. For other mesoderm cell types tried, cardiac, septum transversum, and gastric/esophageal mesenchyme resulted in failure to produce an organoid composition exhibiting one or more of the above aspects. All attempts using Day 9 hAGO resulted in failure. Epithelial cells in different configurations were tested: as spontaneous spheroids that form during definitive endoderm differentiation, aggregated spheroids, and suspensions of single cells generated by dissociation of spontaneous gut spheroids and/or or monolayers. Spontaneous gut spheroids contain about 1,000 cells per spontaneous gut spheroid. Aggregated spheroids contain about 3,000 epithelial cells per aggregated spheroid.

TABLE 1
Correlation of Different Cellular Recombination Parameters

| Outcome | Success | Failure | Failure | Failure | Failure | Failure | Failure |
|---|---|---|---|---|---|---|---|
| What Day of hAGO | Day 6 | Day 6 | Day 6 | Day 6 | Day 6 | Day 6 | Day 9 |
| protocol | | | | | | | |
| With Neural Cells | Yes | No | Yes | Yes | Yes | Yes | Yes |
| With Splanchnic | Yes | Yes | No | No | Yes | Yes | Yes |
| Mesoderm Cells | | | | | | | |
| With Other | No | No | No | Yes | No | No | No |
| Mesoderm Cell | | | | | | | |
| Types | | | | | | | |
| With Spontaneous | Yes | Yes | Yes | Yes | No | No | Yes |
| Epithelial Spheroids | | | | | | | |
| With Aggregated | No | No | No | No | Yes | No | No |
| Epithelial Spheroids | | | | | | | |
| With Single | No | No | No | No | No | Yes | No |
| Epithelial Cells | | | | | | | |
| Ratio of Cell Types | 2.5:1.5-2.5:1 | 2.5:1.5-2.5 | 1.5-2.5:1 | 2.5:1.5-2.5:1 | 2.5:4.5-7.5:1 | 3:2:1 | 2.5:1.5-2.5:1 |
| (mesenchyme:epithelium:neural) | | | | | | | |

### EXAMPLES

### Example 1. Deriving Mesenchyme from hPSCs and Incorporation into Gastric Organoids

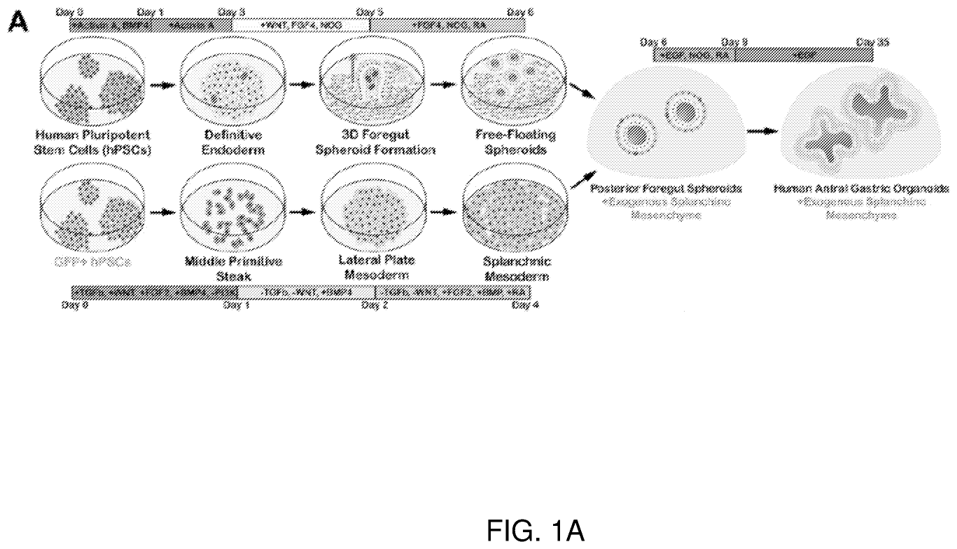

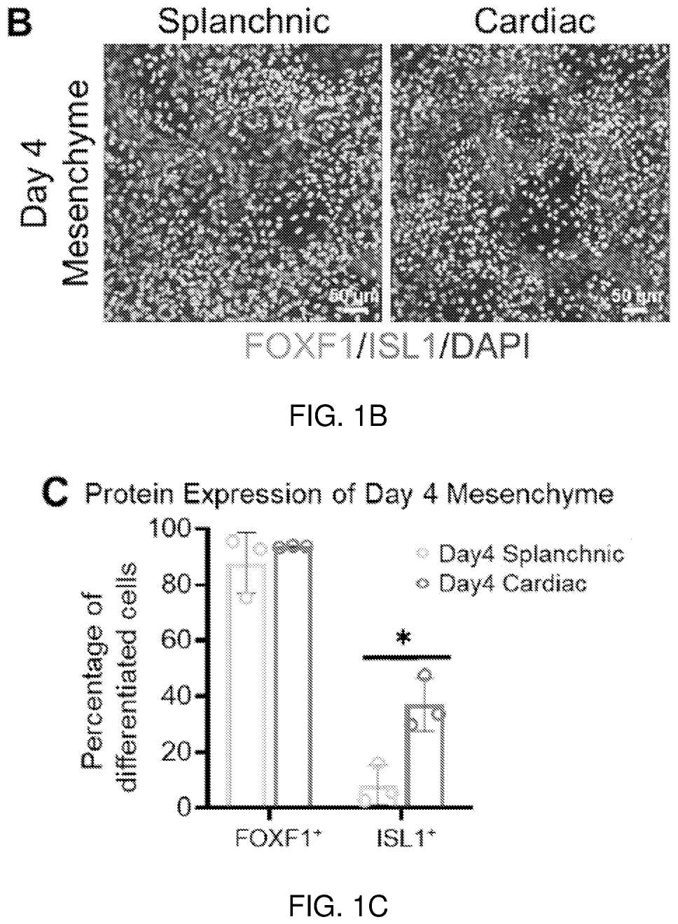

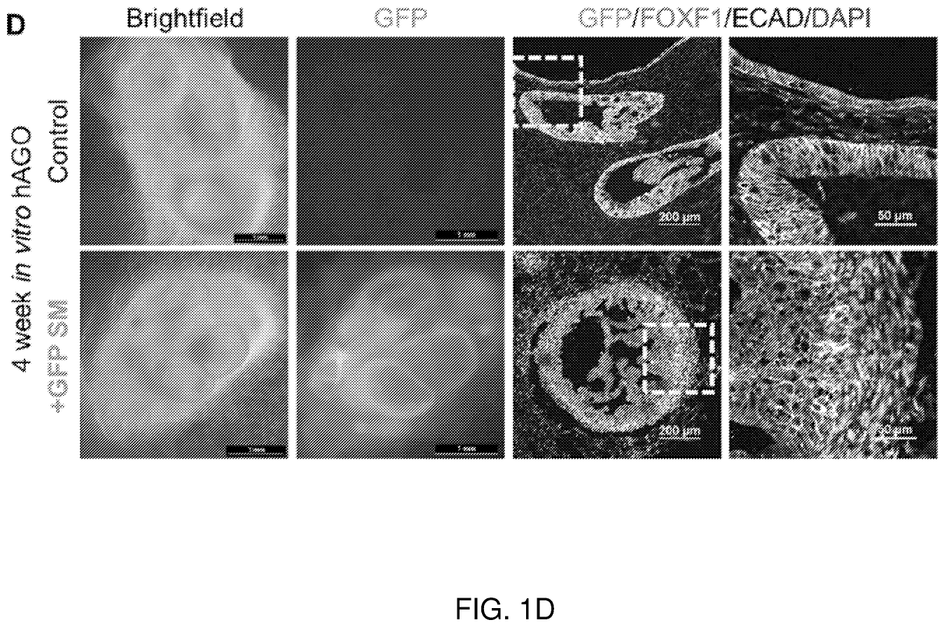

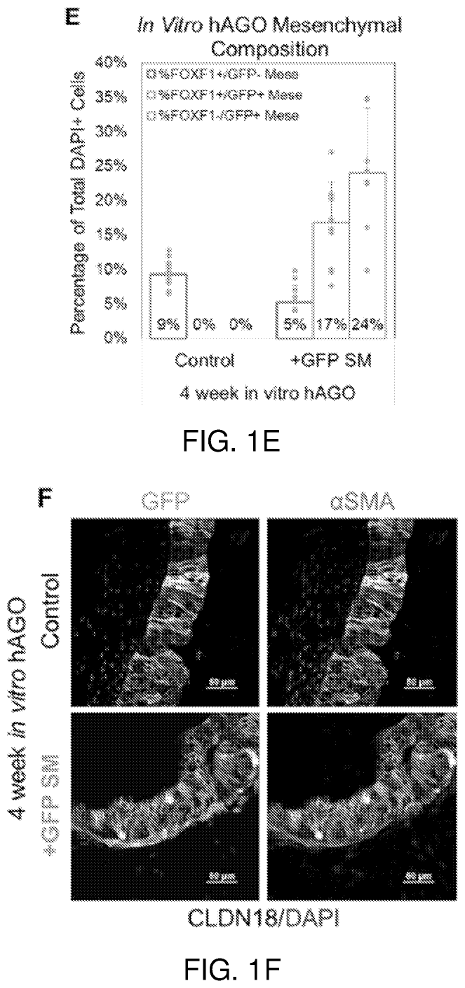

One of the first and most important steps in GI development is the assembly of epithelium and mesenchyme into a primitive gut tube. Establishing this basic epithelial-mesenchymal structure is essential for all subsequent stages of GI development. While PSC-derived human gastric organoids have a full complement of epithelial cell types, they do not intrinsically develop a robust mesenchyme. Therefore, an approach was developed to generate GI mesenchyme from hPSCs that could be incorporated into gastric organoids. Previous work identified a method to direct the differentiation of hPSCs into splanchnic mesenchyme (SM), the source of gastric mesenchyme. This method was based on the signaling pathways that drive the normal development of GI mesenchyme and yields a robust population of SM. Briefly, hPSCs were differentiated into lateral plate mesoderm (LPM) with TGFβ inhibition, WNT inhibition, and BMP activation as previously published (FIG. 1A). As LPM can give rise to both cardiac mesoderm and SM, the LPM was treated with retinoic acid (RA) to induce a SM fate, resulting in loss of cardiac markers and an increase in expression of SM markers like FOXF1 (FIG. 1B-C). Methods of producing splanchnic mesenchyme are explored in PCT publication WO 2021/041443, which is hereby expressly incorporated by reference in its entirety.

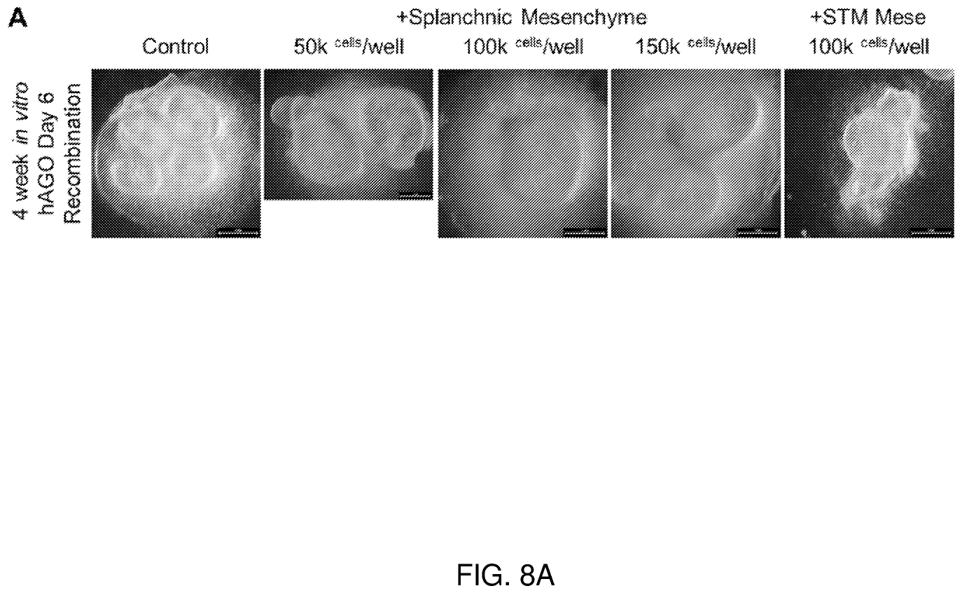

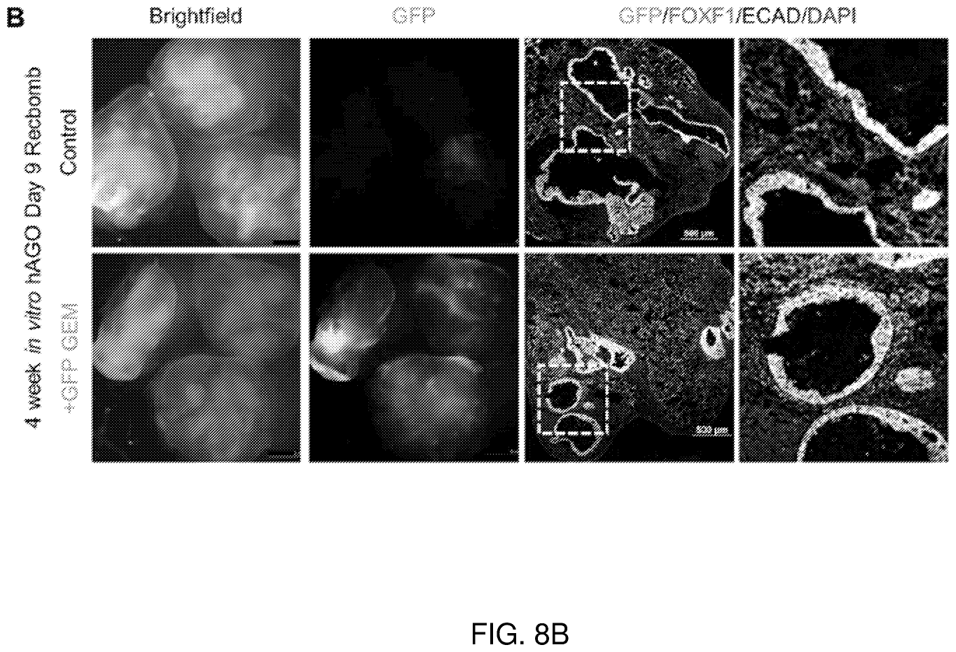

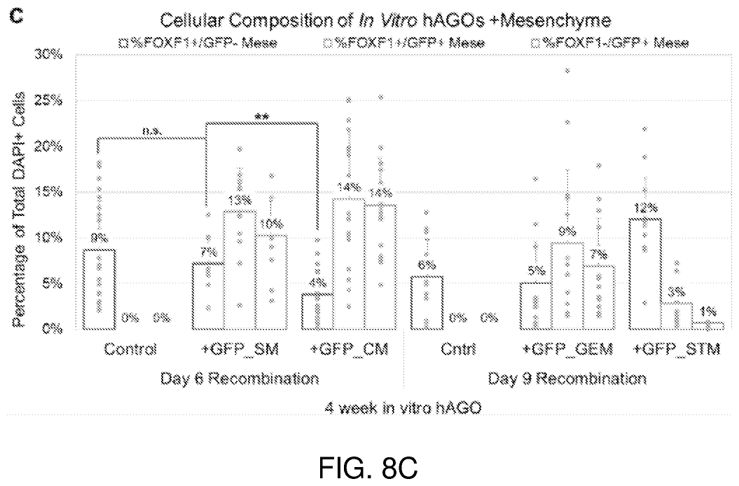

Several approaches to incorporate mesenchyme into developing gastric organoids were investigated, including combining mesenchyme and epithelium at different epithelial developmental stages (e.g. at either day 6 or day 9 of the hAGO protocol), testing a single cell aggregation method versus using intact epithelial organoids, and utilizing differently patterned mesenchymal populations, including splanchnic, cardiac, septum transversum, and gastric-esophageal mesenchyme. To monitor this mesenchymal incorporation in real time, the mesenchyme was derived from an hPSC line with constitutive GFP expression. It was observed that starting with a single cell suspension of splanchnic mesenchyme that was aggregated with intact posterior foregut spheroids on day 6 of the hAGO protocol (FIG. 1A) resulted in optimal mesenchymal incorporation that yielded the most added exogenous mesenchyme while still retaining the small portion of endogenous mesenchyme (FIG. 8A-C). To determine this, incorporation of varying concentrations of SM and septum transversum (STM) mesenchyme on day 6 of the hAGO protocol were initially compared. Visual qualitative assessment of brightfield images of 4 week in vitro hAGOs recombined with either SM or STM mesenchyme showed that recombining SM with hAGO spheroids at a concentration of 50,000 cells/well of approximately 20-30 hAGO spheroids (equating to an approximate 2:1 ratio of SM cells to hAGO epithelial cells) resulted in end timepoint hAGOs +SM that retained an epithelium of visually similar size to hAGO controls (FIG. 8A). Subsequently, 4 week in vitro hAGOs that were recombined with either SM on day 6 of the hAGO protocol or regionally pattered gastric-esophageal mesenchyme (GEM) on day 9 of the hAGO protocol were compared (FIG. 1D, FIG. 8B). After 4 weeks growth in vitro, hAGO +SM had a robust and uniform layer of GFP+ mesenchymal cells expressing the early SM marker FOXF1 surrounding the gastric epithelium (FIG. 1D), while hAGO +GEM still showed a nonuniform layer of GFP+ mesenchyme (FIG. 8B). In gastric organoids+SM, a third to a half of all cells were mesenchyme, representing a 3-5-fold increase over control organoids without added mesenchyme (FIG. 1E, 8C). However, in hAGOs +GEM, only about a fourth of all cells were mesenchyme (FIG. 8C). This was even less for hAGOs +STM (FIG. 8C). Overall, mesenchymal populations of SM and CM recombined on day 6 of the hAGO protocol yielded more GFP+ and GFP+/FOXF1+ mesenchyme in 4 week in vitro hAGO cultures than populations of GEM and STM that were recombined on day 9 of the hAGO protocol (FIG. 8C). Then, between SM and CM populations recombined on day 6, hAGOs +SM retained more endogenous FOXF1+ mesenchyme than hAGOs +CM (FIG. 8C). Taken together, it was determined that SM recombined with posterior foregut spheroids on day 6 of the hAGO protocol resulted in mesenchymal incorporation that yielded the high populations of both endogenous and exogenous mesenchyme. Finally, in hAGOs +SM, very rare GFP+ mesenchymal cells that showed the capacity to differentiate in vitro into αSMA+ smooth muscle were observed (FIG. 1F). Otherwise, mesenchymal cells did not differentiate in vitro into αSMA+ smooth muscle and this process only occurred after transplantation onto a vascular bed in immunocompromised mice.

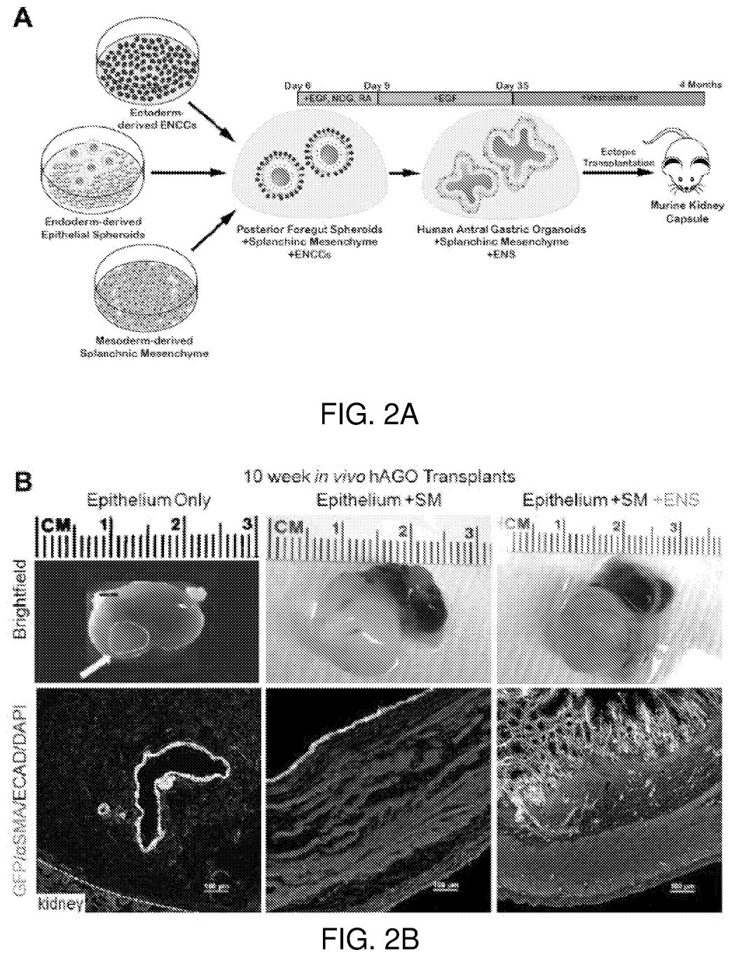

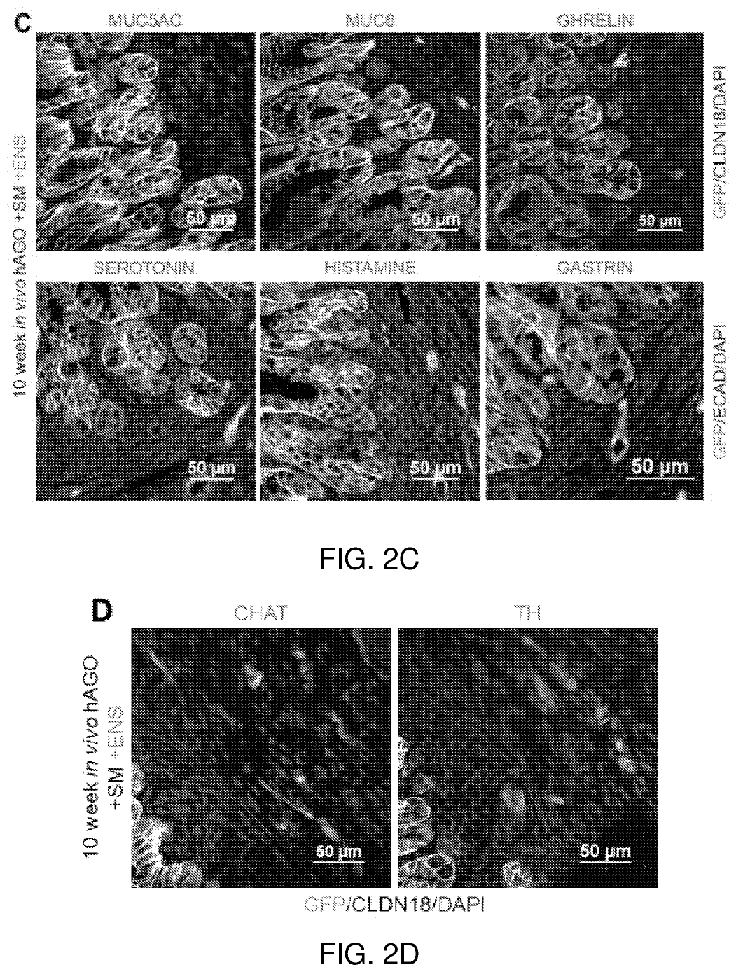

Similarly, other aspects of GI organoid growth and morphogenesis are also limited in vitro but upon transplantation, intestinal and colonic organoids continue their growth and maturation. To fully investigate which organoid engineering approach would yield the most characteristic stomach tissue containing glandular units of simple columnar epithelium surrounded by multiple layers of differently oriented smooth muscle layers, all organoids were transplanted into mice and grown for an additional 10 weeks. Only one organoids of a few mm in size was transplanted into each mouse. Most hAGOs without added mesenchyme did not survive (˜60%) and those that did were small containing only a simple, one-cell layer of thin cuboidal-like epithelium that did not fully differentiate (FIG. 2B). In contrast, hAGOs engineered with exogenous mesenchyme had a high survival rate (100%) and grew from a few millimeters to 0.5-1.5 cm in diameter, exhibiting up to 1000× increase in volume in some cases. The mesenchyme differentiated into layers of poorly organized αSMA+ smooth muscle that surround a simple layer of hAGO epithelium (FIG. 2B). This data suggests that the incorporation of exogenous mesenchyme promotes engraftment and growth of hAGOs but does not result in normal gastric tissue with glandular units of simple columnar epithelium.

### Example 2. Engineering Three Germ Layer Human Gastric Tissue

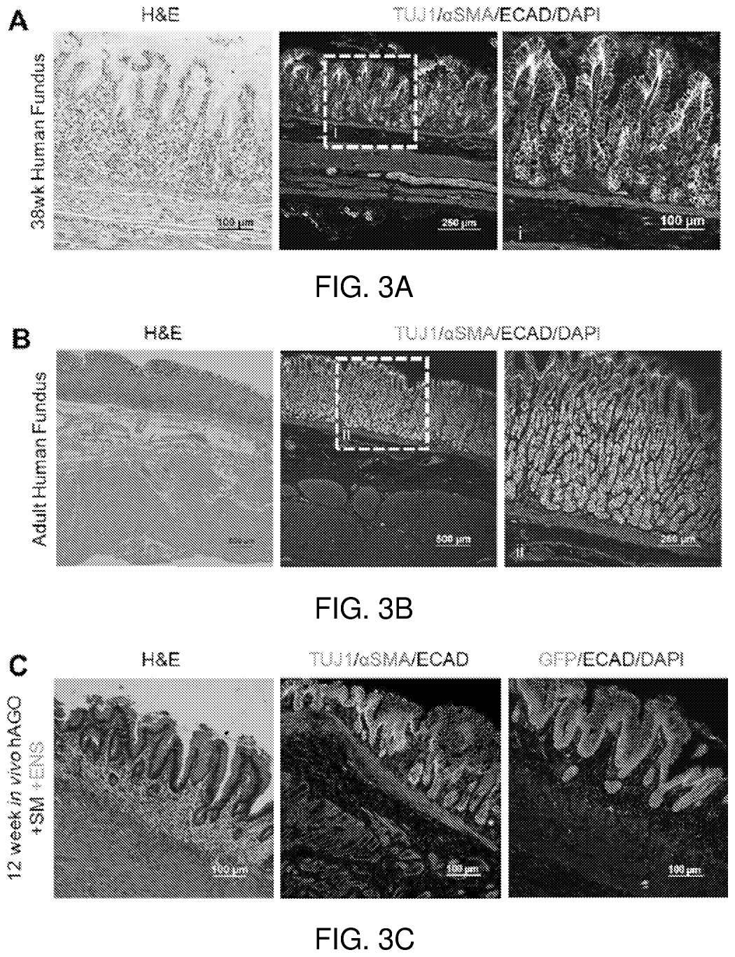

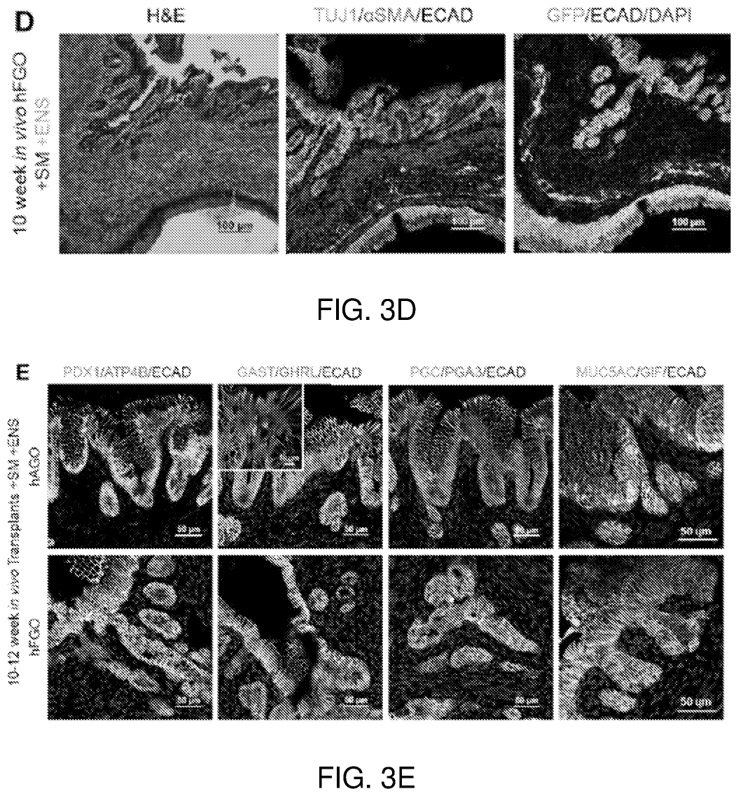

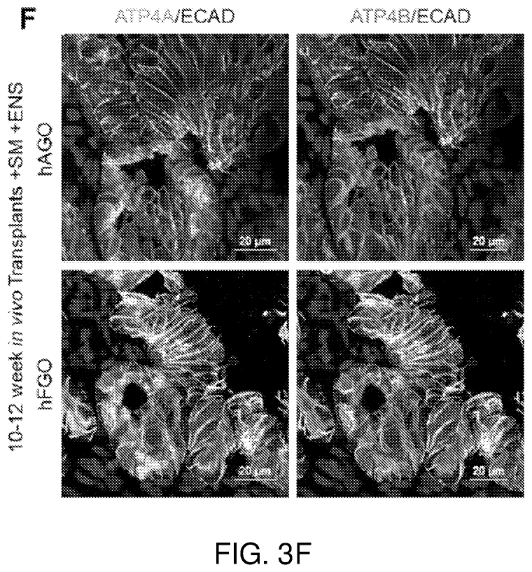

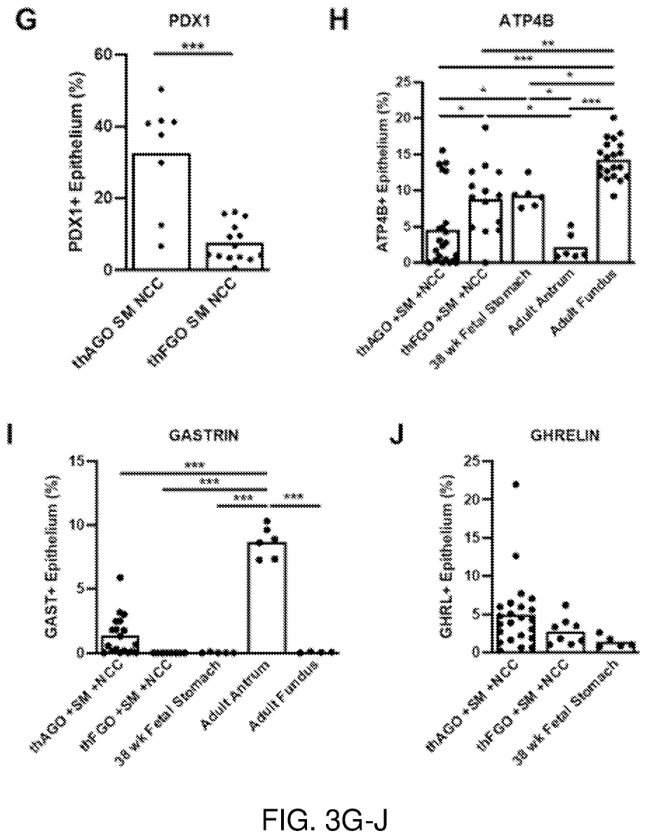

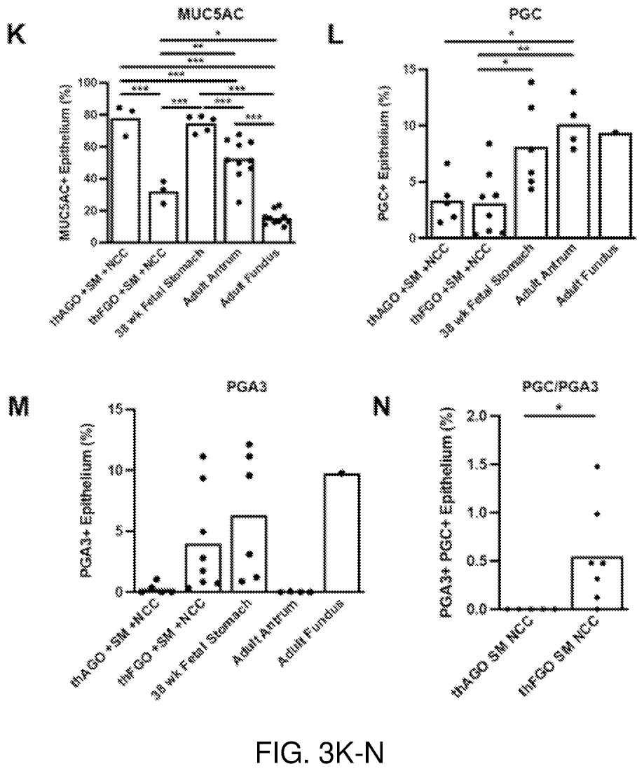

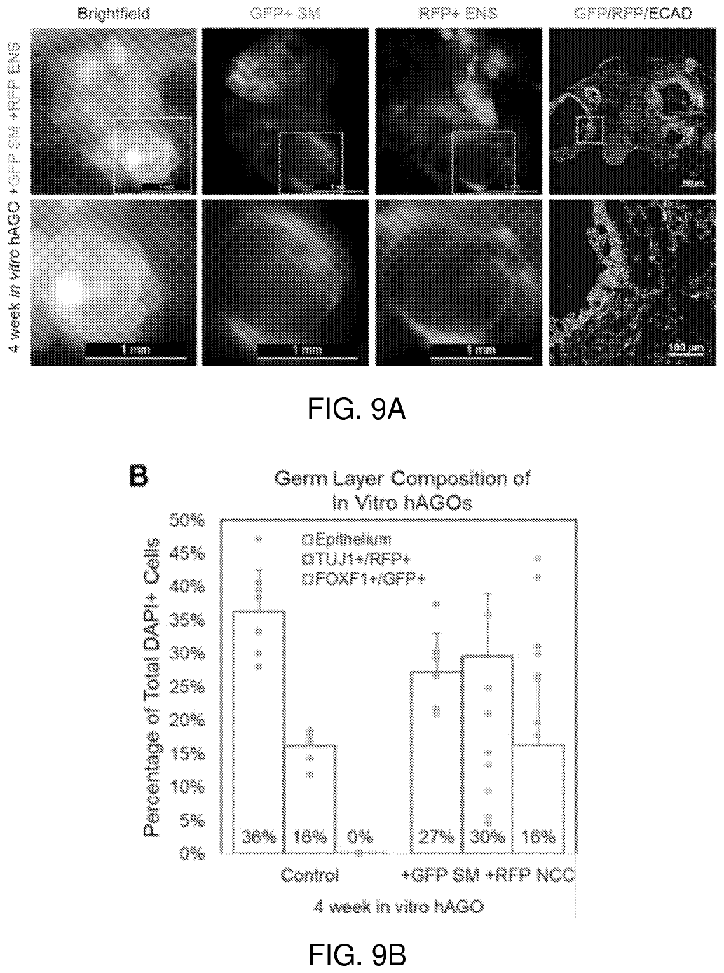

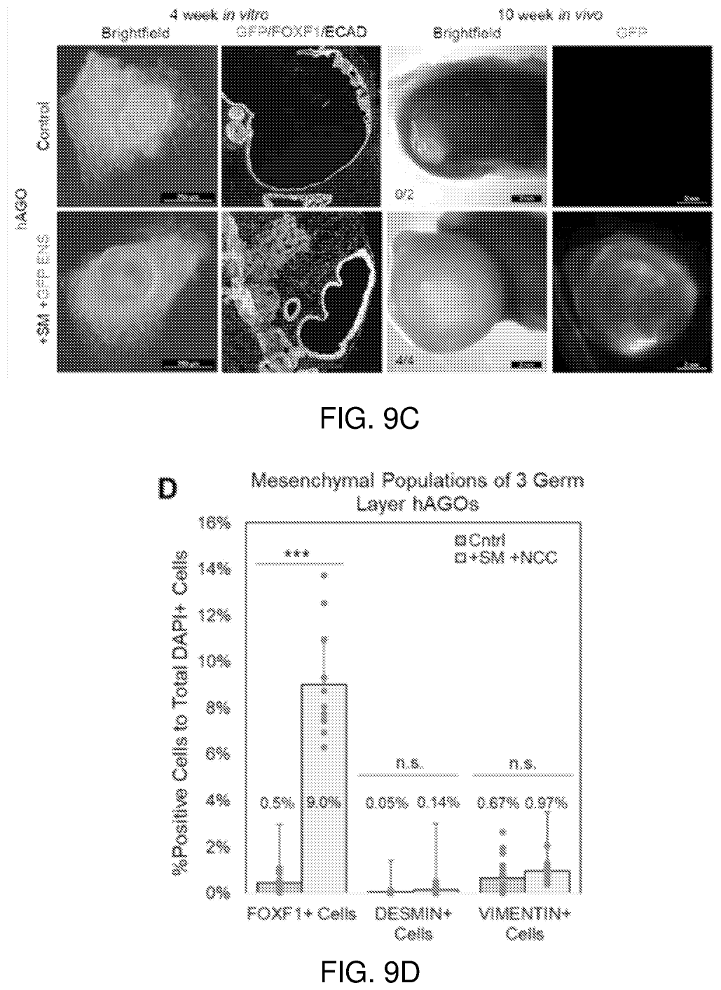

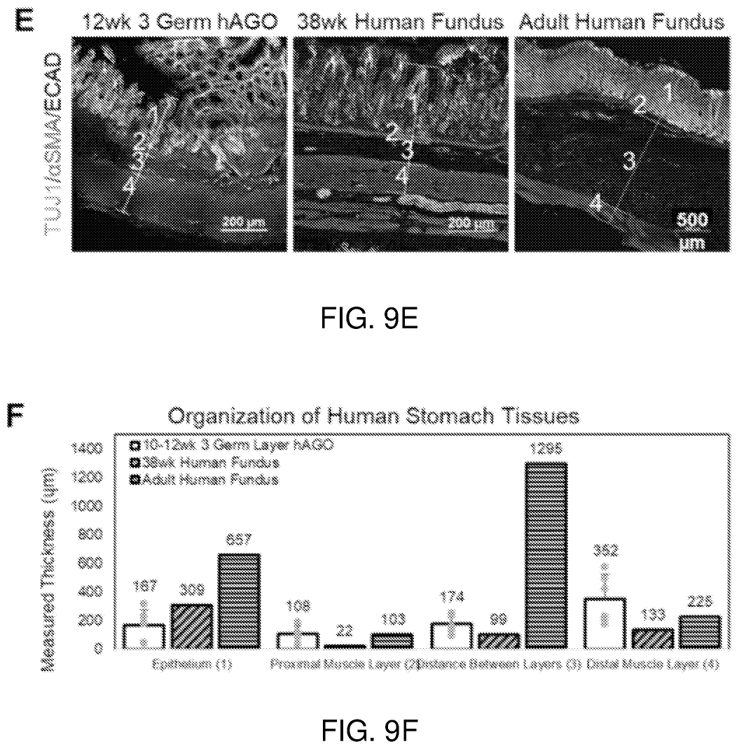

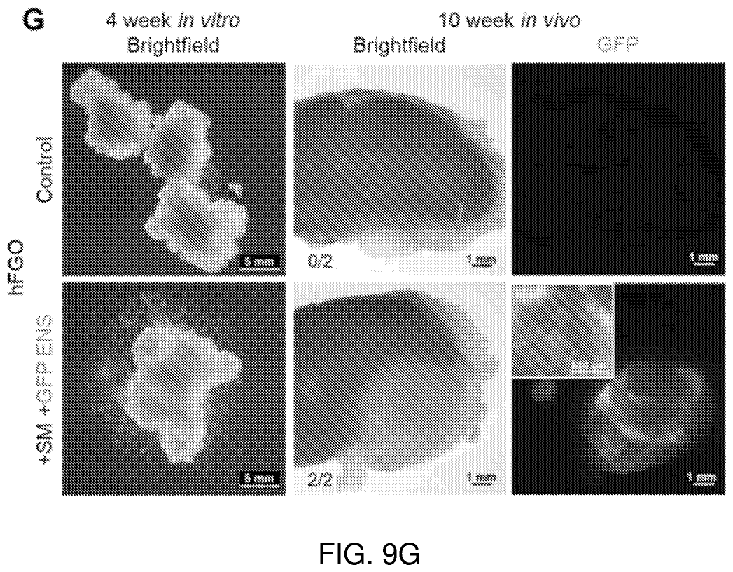

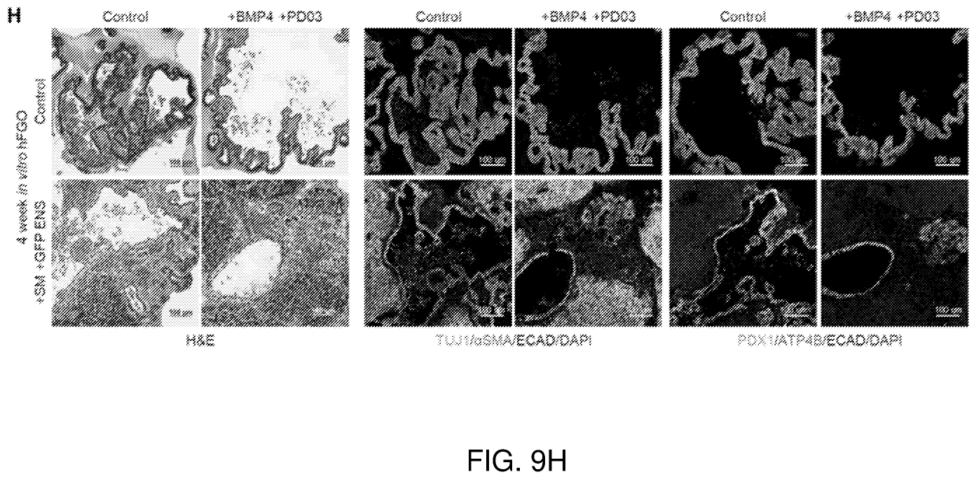

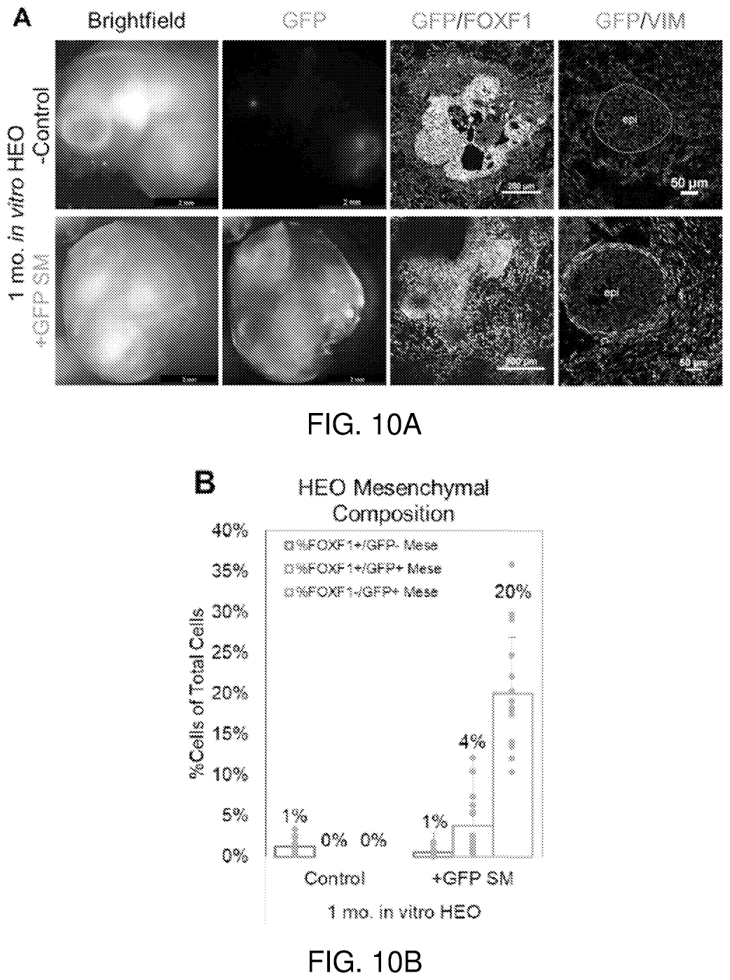

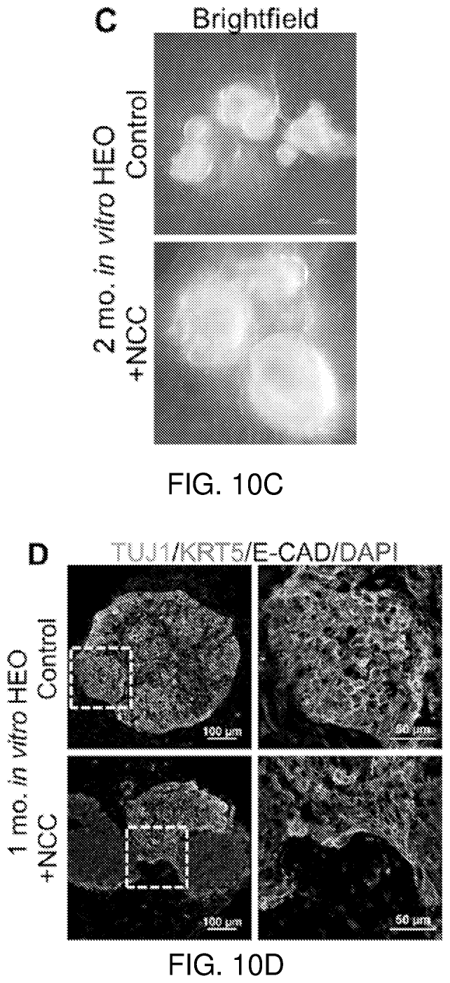

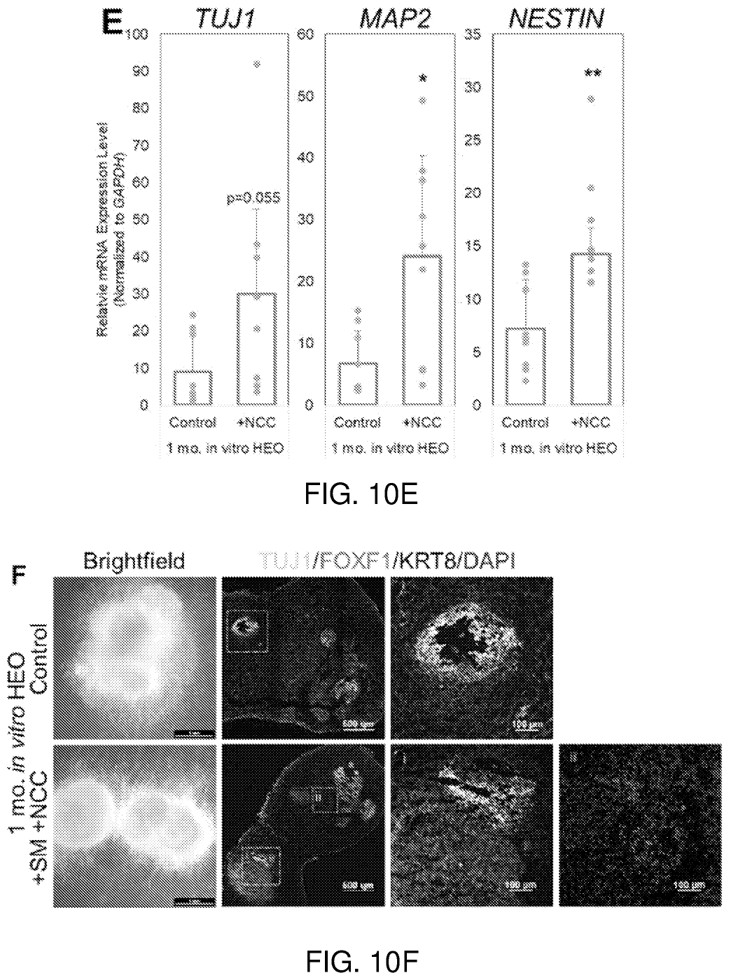

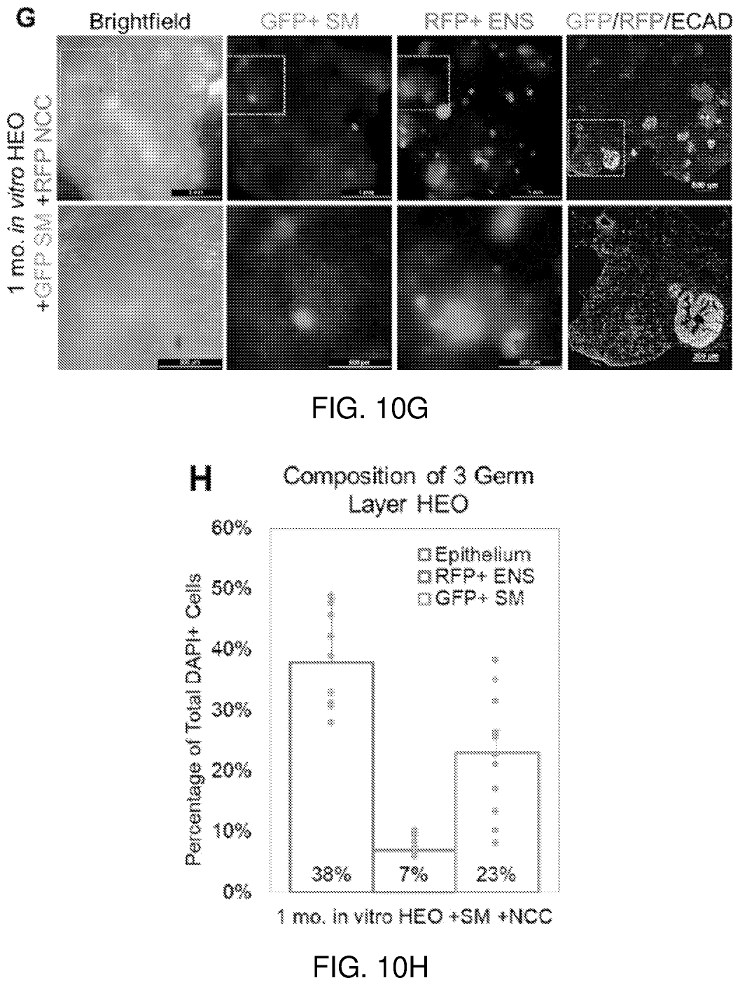

While SM improved the growth of hAGOs, with the smooth muscle was poorly organized and there was no evidence of epithelial development into the glandular structures that normally form during human stomach organogenesis. Therefore, it was investigated if incorporation of ectodermally-derived ENCCs, in combination with SM, into hAGOs would result in normal stomach development (FIG. 2A). ENCCs migrate into the developing gut tube early in development and form a neuroglial plexus called the enteric nervous system (ENS). To recapitulate the spatiotemporal dynamics of this developmental process, ENCCs were differentiated as previously described and were aggregated with hAGO spheroids, along with SM. By using RFP-labeled ENCCs and GFP-labeled SM, conditions were identified that allowed for the incorporation of both germ layers into hAGOs in vitro (FIGS. 9A and 10G). To allow for organoid growth and maturation, the recombined hAGOs were transplanted into mice for 10-12 weeks. While both hAGOs +SM and hAGO +SM +ENS transplants grew to over 1 cm in diameter, only hAGO +SM +ENS recombinants formed the stereotypic glandular structures found in the human stomach (FIGS. 2B and 3A-B). In addition, several distinct layers of highly organized smooth muscle that were orientated into distinct layers similar to the organization of the muscularis mucosa, submucosa, and muscularis externa, containing the inner circular and outer longitudinal layers of smooth muscle, of the stomach, were observed (FIG. 2B, 3A-B, 9E-F). The 12 week three germ layer hAGOs are more similar in organization to 38 week human stomach tissue (FIG. 3A) when compared to adult human stomach tissue (FIGS. 3B and 9E-F). Embedded within the smooth muscle fibers was a network of enteric neurons arranged in characteristic plexi (FIG. 2B). The first, more proximal, plexus layer in the 12 week three germ layer hAGO lies in between the more proximal muscularis mucosa-like muscle layer and the more distal, muscularis externa-like muscle layer, essentially within a submucosal-like space. This plexus layer then is spatially similar to the submucosal neuronal plexus of human stomach tissue. The second, more distal, plexus layer is embedded within the muscularis externa-like muscle layer, mimicking the organization of the myenteric plexus. This organization is highly similar to in vivo 38 week stomach tissue (FIG. 3A, 9E-F).

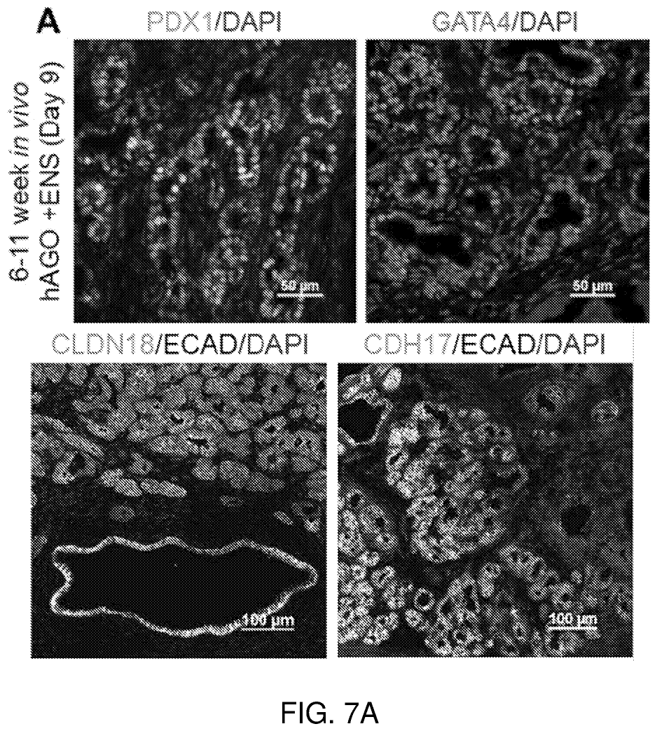

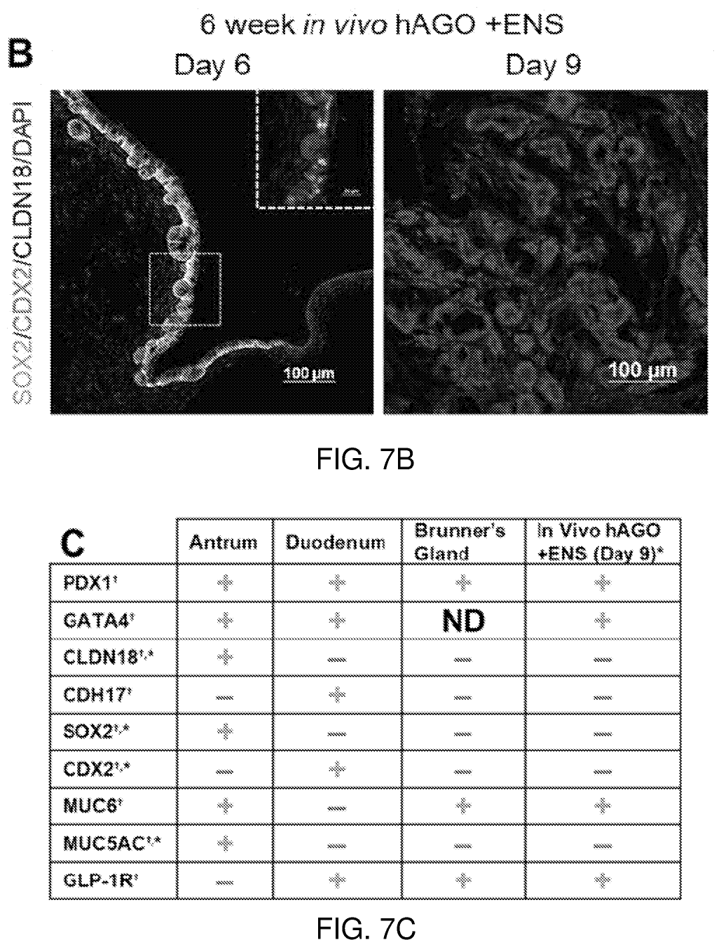

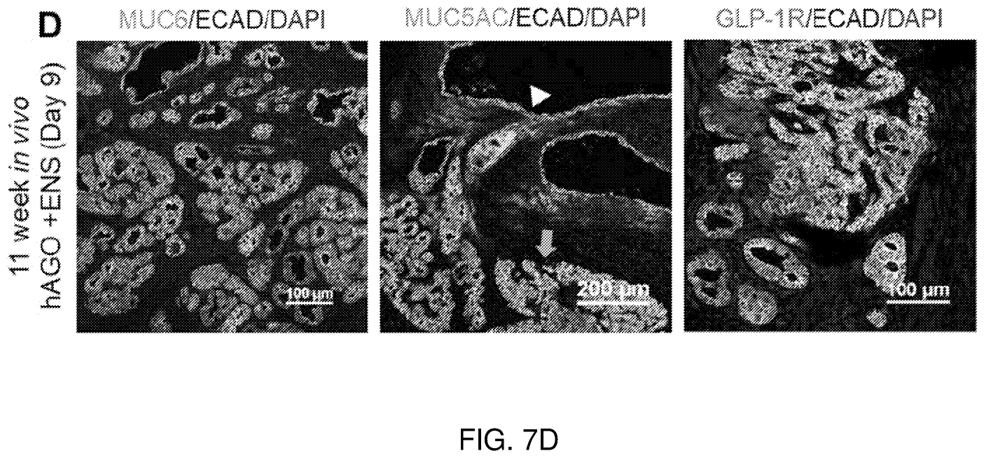

The epithelium of hAGO +SM +ENS transplants expressed the gastric epithelial marker CLDN18 and lacked the intestinal epithelial marker CDH17, confirming the gastric identity of the organoids (FIG. 7A). hAGO glands contained all of the expected cell types normally found in the antrum of the stomach including surface mucous cells (MUC5AC), gland mucous cells (MUC6), and endocrine cells expressing ghrelin, serotonin, histamine, and gastrin (FIGS. 2C and 3E). The neurons (GFP+) formed a network of fibers that resembled a plexus and was embedded within the layers of smooth muscle. GFP+ choline acetyltransferase+(ChAT+) and dopaminergic (TH+) neurons in close proximity to the glandular epithelium and endocrine cells were observed (FIG. 2D). This association in vivo is important, as neurotransmitters control secretion of a variety of stomach hormones including ghrelin and gastrin.

### Example 3. Generating Fundic Tissues Containing Three Germ Layers

One of the most prominent domains of the human stomach is the corpus, which contains fundic (oxyntic) glands with acid producing parietal cells and chief cells that secrete digestive enzymes. The glands are also in close proximity to enteric neurons that, along with gastric endocrine cells, help to regulate acid production. It was investigated if the three germ layer recombinant approach could also be used to engineer human fundic tissue with the above properties. Early stage hFGOs were generated and recombined with SM and GFP-labeled ENCCs. After four weeks, both SM and ENCCs incorporated into hFGOs (FIG. 9G), similar to hAGOs (FIG. 9A-D). Fundic identity was confirmed by the presence of ATP4B+ parietal cells (FIG. 3E-F).

Next, it was investigated whether, like hAGOs, incorporation of ENCCs and SM also promoted growth, morphogenesis, and maturation of hFGOs engrafted under the murine kidney capsule (FIG. 9G). A comparison of three germ layer hAGOs and hFGOs grown in vivo for 10-12 weeks showed that they both grew up to a centimeter in size with a histology much more similar to 38 week (FIG. 3A) and adult (FIG. 3B) human antral tissues, with glandular epithelium surrounded by multiple layers of innervated smooth muscle (FIG. 3C-D). In general, the extent of glandular morphogenesis of hFGOs was less than that of hAGOs (FIG. 3C-D). Both hAGOs and hFGOs maintained their regional identity after transplantation, with hAGOs expressing higher levels of PDX1 and the antral-specific endocrine hormone gastrin, with hFGOs lacking these markers (FIG. 3E). Moreover, the proportions of cell types that normally distinguish the human corpus/fundic from the antrum also distinguished hAGOs from hFGOs. Specifically, hFGOs contained more ATP4B+/GIF+ parietal cells than the hAGOs, and hFGOs had fundic-specific PGA3+ chief cells in addition to progastricsin (PGC)-expressing chief cells that are observed in both regions of the stomach (FIG. 3E, 9H). Formation of chief and parietal in vitro requires the use of BMP and MEK inhibitors; however, transplanted hFGOs required no additional factors for robust parietal and chief cell differentiation (FIGS. 3E and 9G-H), demonstrating that the signaling processes that control gastric cell type specification occur normally in engineered tissue. As observed in human stomach biopsies, engineered antral tissue does contain parietal cells, but at lower numbers than are found in fundic glands (FIG. 3E, 9H). Furthermore, the cellular localization of ATP4B to the apical membrane suggests these parietal cells are more mature than their in vitro counterparts. Quantification of distinct layer thicknesses of hAGO compared to human fundus tissue is depicted in FIG. 9F. Together, these data confirm that engineering mesenchyme and ENS cells into hAGOs and hFGOs results in the formation of gastric tissues that resemble human stomach tissue.

### Example 4. Antral Three Germ Layer Organoids Exhibit Functional Muscle Contraction

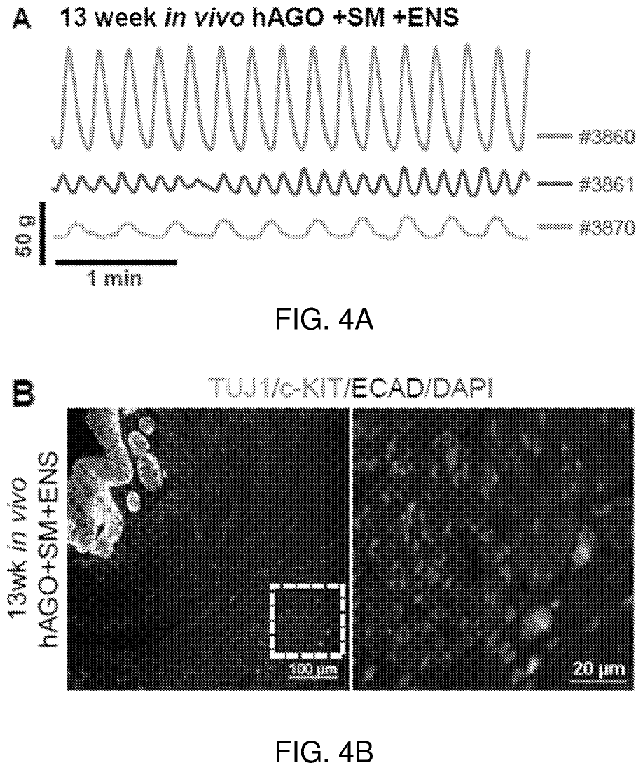

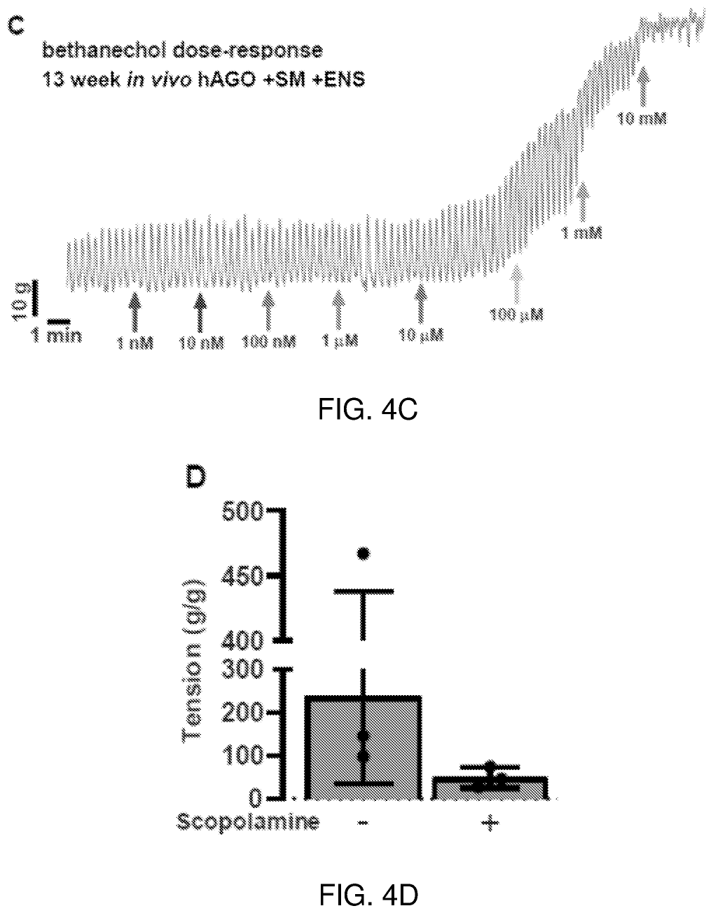

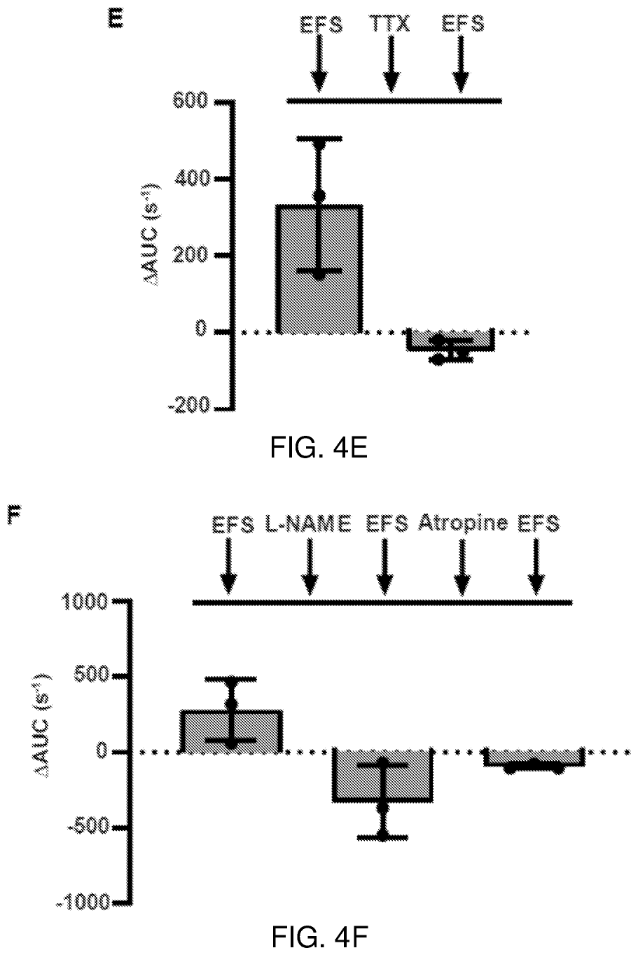

The stomach plays an essential role in the mechanical breakdown of food and in emptying it into the duodenum. This gastric motility involves the ENS, which functionally controls smooth muscle contractions. To investigate if the ENS and smooth muscle in the three germ layer hAGOs formed a functional neuromuscular unit, tissue strips were isolated from transplanted hAGOs and placed in an organ bath chamber system to monitor contractility. After an equilibration period, spontaneous contractile oscillations were observed from tissues derived from 3 separate hAGO +SM +ENCC transplants (FIG. 4A). The presence of regular phasic contractions indicated that intramuscular interstitial cells of Cajal (ICCs) were present within the organoids. This was further supported by the presence of mesenchymal clusters expressing KIT Proto-Oncogene, Receptor Tyrosine Kinase (c-KIT), a marker of ICCs, that were in close association with GFP+ and TUJ1+ neuroglia cells (FIG. 4B), indicating cooperative coordination of the contractions. These mesenchymal clusters arranged within the muscularis externa-like muscle layers of the three germ layer hAGO transplants. Smooth muscle force contraction was interrogated with a dose response to bethanechol, a muscarinic receptor agonist that directly stimulates smooth muscle contractions (FIG. 4C). The contractility increased in response to bethanechol in a dose-dependent manner, demonstrating the presence of functional smooth muscle. Moreover the contractions were able to be reversed and muscle relaxation induced with addition of scopolamine, a muscarinic antagonist. Quantification showed that the normalized mean maximal tension was 236.9 g/g, while the mean minimal tension was 49.3 g/g (FIG. 4D), indicative of functional muscle tissue in the in vivo engrafted hAGOs.

It was investigated if the ENS that was engineered into hAGOs was functionally capable of controlling gastric tissue contractions. Electrical field stimulation (EFS) of tissues is an experimental means to trigger neuronal firing and subsequent smooth muscle contraction. EFS pulses were administered to hAGO tissue and resulted in an increase in contractile activity, indicating that the ENS was regulating smooth muscle. To show that there was a functional connection between the ENS and smooth muscle, ENS activity was inhibited with the neurotoxin tetrodotoxin (TTX), which abolished the ability of EFS to stimulate contractile activity (FIG. 4E). Lastly, the involvement of nitrergic and cholinergic neuronal activity in regulating smooth muscle contractions was investigated. Nitric oxide synthetase (nNOS)-expressing neurons were inhibited with NG-nitro-l-arginine methyl ester (L-NAME), a nitric oxide synthesis inhibitor, and cholinergic neurons were inhibited using atropine, an acetylcholine (Ach) receptor antagonist. Contractile activity was measured following control stimulation and stimulation after compound exposure, and was expressed as the change in the area under the curve (AUC) immediately before and after each EFS stimulation (FIG. 4F). These data provide insight into the proportions of nitrergic and cholinergic neuronal activity compared to the total ENS activity (control EFS) and show that gastric tissue contractions involved both nitrergic and cholinergic neuronal activities.

### Example 5. Three Germ Layer Esophageal Organoids

To test whether the approach of combining tissue from three germ layers was broadly applicable to engineering other organs, SM and ENCCs were incorporated into developing human esophageal organoids (HEOs). Like hAGOs and hFGOs, GFP-labeled SM was added HEOs that are initially largely epithelial (FIG. 10A-B). After four weeks, in vitro HEOs+SM had a robust layer of GFP+ mesenchyme surrounding the epithelium (FIG. 10A) with a high percent of these co-expressing FOXF1 or the more differentiated marker vimentin (VIM) (FIG. 10A). Quantification showed that control HEOs only contain ˜1% of endogenous mesenchyme while HEOs+SM contain ˜25% mesenchymal cells (FIG. 10B). Interestingly, the addition of exogenous GFP+ mesenchyme facilitated the expansion of endogenous FOXF1+/GFP-mesenchyme in the cultures, suggesting cell-cell interactions that promote the growth and development of both the organoid epithelium and mesenchyme.

Next, ENCCs were incorporated into HEOs with or without SM (FIG. 10C-H). After 1 month of in vitro culture, the ENCCs in HEOs without SM had differentiated into TUJ1/MAP2/NESTINM+ enteric neurons that aggregated tightly around the epithelium and did not organize into a neuronal plexus (FIG. 10D-E). In contrast, when both ENCCs and splanchnic mesenchyme were recombined with HEO epithelium, robust co-development of TUJ1+ neuronal plexus associated within FOXF1+ mesenchymal layer was observed (FIG. 10F-H). Overall, these findings show that different human GI organ tissues can be engineered by combining progenitors from all three germ layers and emphasize the importance of reciprocal cell-cell communication between the epithelial, mesenchymal and ENCCs for proper assembly and function of embryonic organs.

### Example 6. ENCC Differentiation into ENS Neuroglial Cell Types does not Require the Addition of Exogenous Mesenchyme

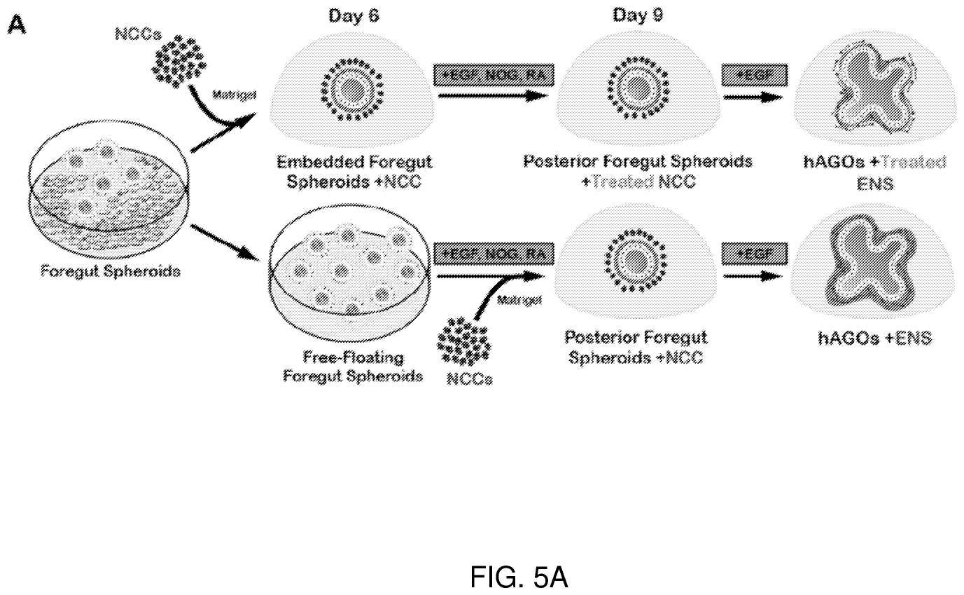

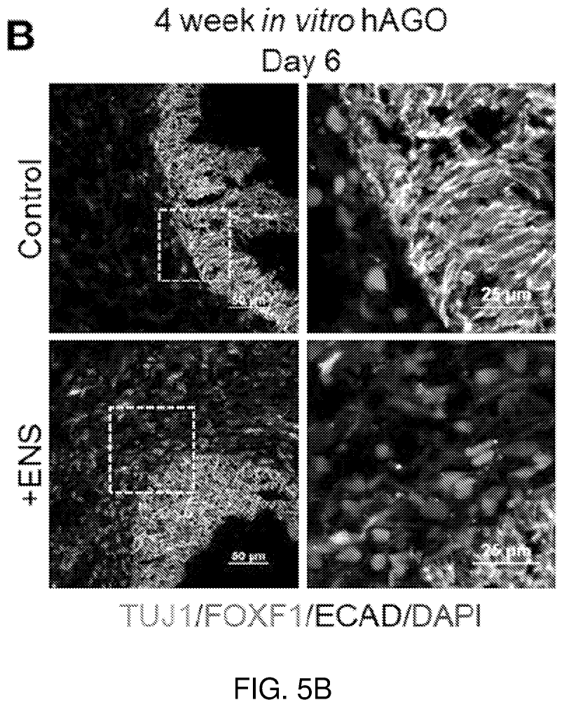

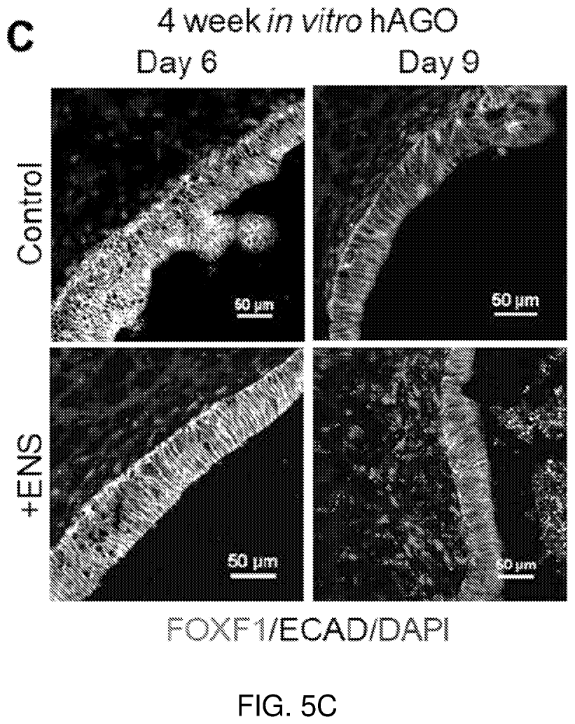

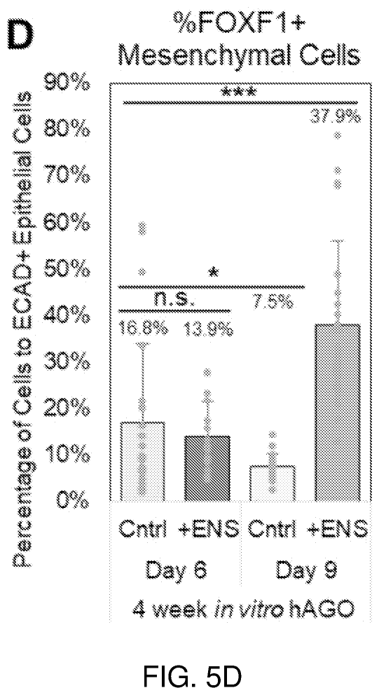

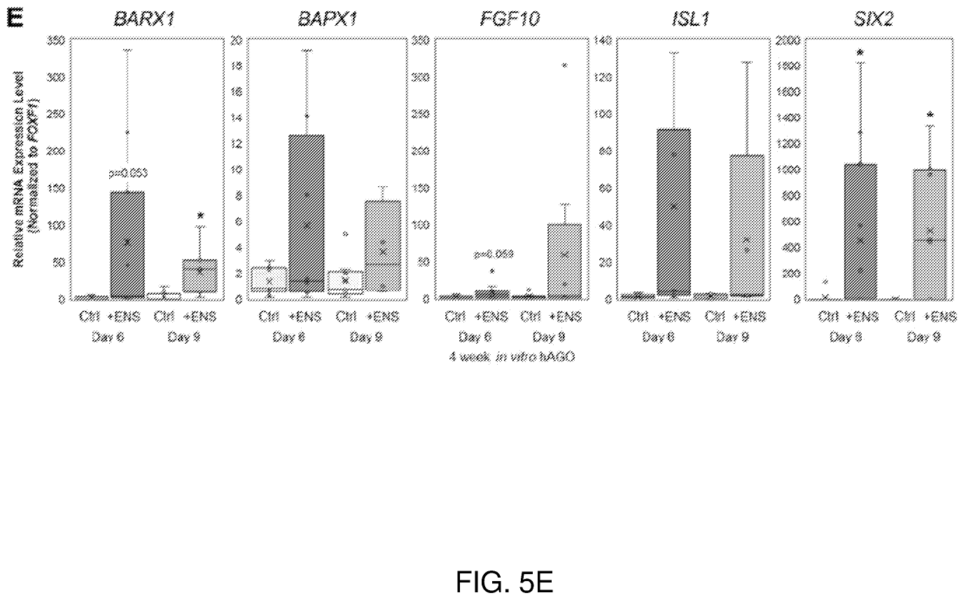

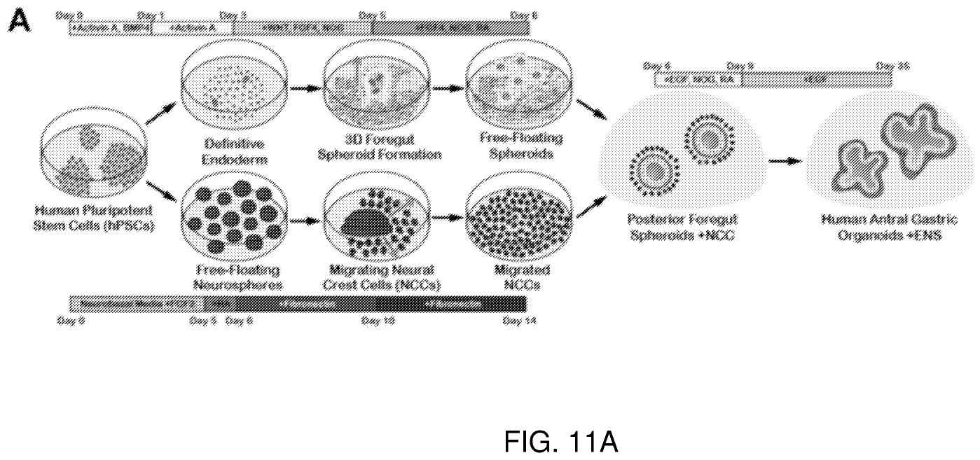

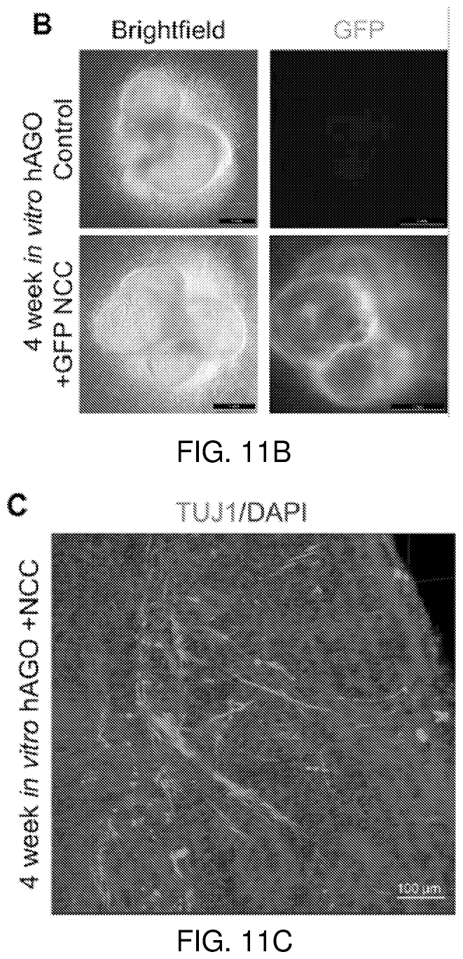

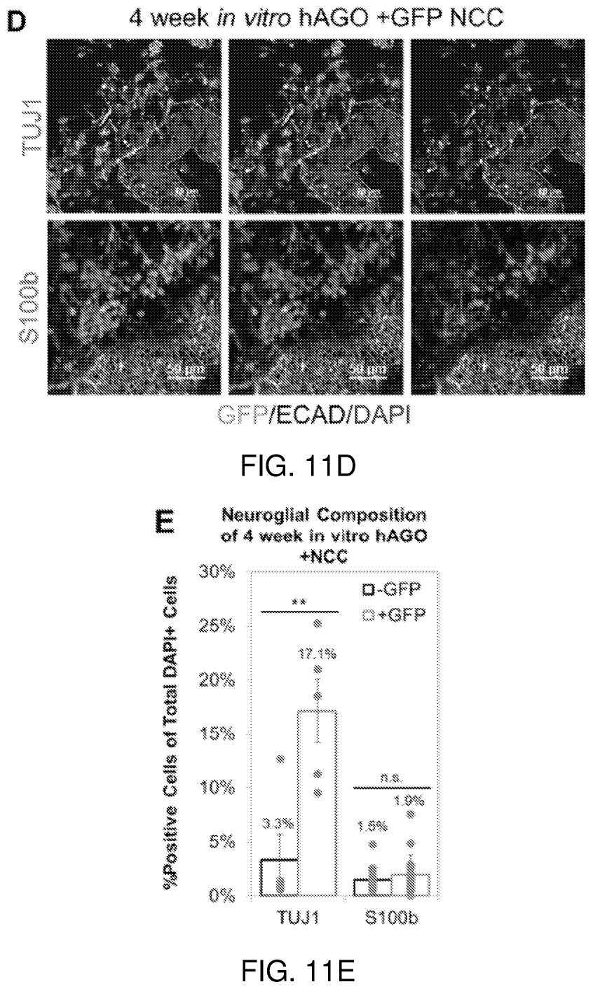

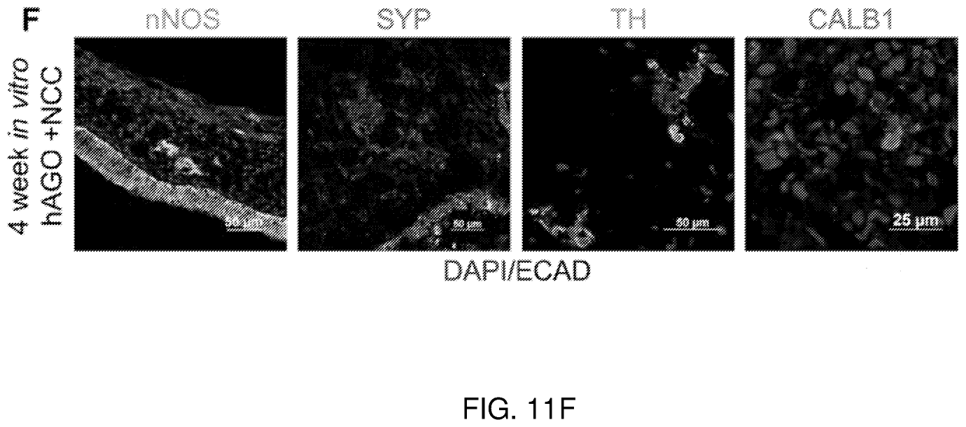

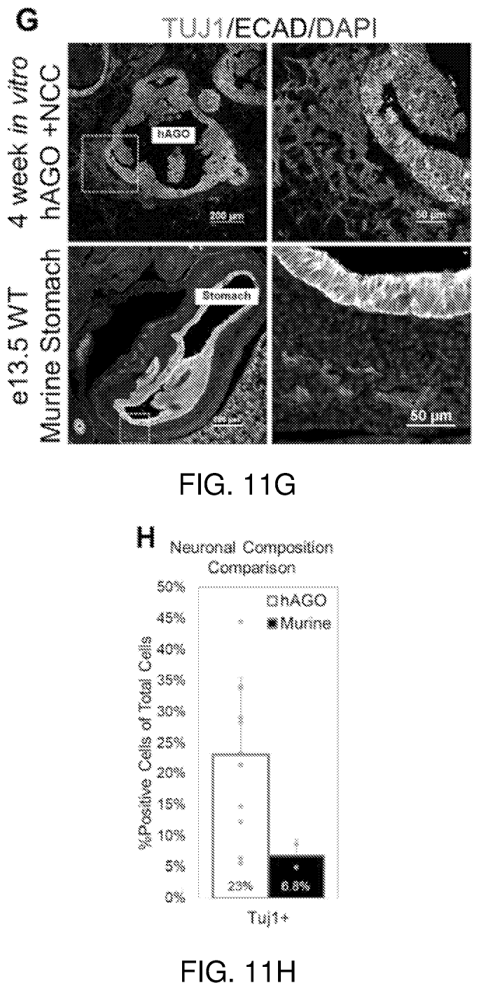

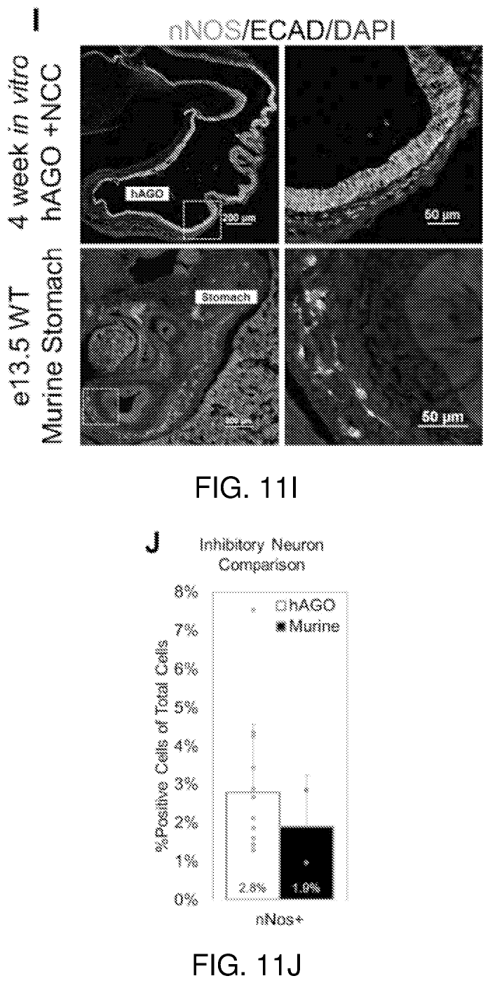

One of the most powerful aspects of this system is the ability to study interactions between cell types of different germ layers that drive normal tissue formation. For example, the presence of ENCCs was important for the development of both the smooth muscle and the gastric epithelium. Without ENCCs, mesenchyme formed a small layer of disorganized smooth muscle and the gastric epithelium failed to undergo glandular morphogenesis. How ENCCs impact the development of other germ layers was interrogated by taking advantage of the ability to add or remove germ layers at will. First, ENCCs were recombined with hAGOs at two different timepoints, day 6 and day 9 of gastric organoid development, and their ability to form ENS cell types without exogenous mesenchyme was assessed (FIG. 5A). The rationale for recombining ENCCs at day 9 was to avoid exposing ENCCs to retinoic acid (RA) and noggin (NOG) that are in the hAGO cultures between days 6-9. Surprisingly, at either time point, ENCCs incorporated well into hAGOs and formed a 3D network of TUJ1+ neurons and S100b+ glial cells adjacent to gastric epithelium (FIG. 11A-E). ENCCs differentiated into a diverse array of neuroglial subtypes, including inhibitory (nNOS), interneurons (Synaptophysin), dopaminergic (TH), and sensory (Calbindin) neurons (FIG. 11F, Table 2). ENCCs did not alter gross hAGOs growth or morphology after four weeks of development in vitro (FIG. 11B). However, ENS development was abnormal. Neurons were found immediately adjacent to the gastric epithelium and were disorganized as compared to mouse E13.5 embryonic stomach (FIG. 11G-H). There were, however, a comparable number of nNOS+ inhibitory neurons present in hAGOs +ENS compared to mouse E13.5 stomach (FIG. 11I-J). These data show that ENCCs incorporated into hAGOs differentiated into neuroglial subtypes without the addition of exogenous mesenchyme, but that proper spatial orientation and ENS plexus development likely requires a robust population of mesenchyme.

TABLE 2
List of all neural markers assessed within in vitro and in vivo organoid cultures.

| Protein | | | | | |
|---|---|---|---|---|---|
| name used | Protein | Neuroglia cell | | Organoid | Quantification |
| herein | name/type | type | Functionality | expression | for hAGO |
| TUJ1 | Neuron-specific | Differentiated | Microtubule | 4 wk in vitro | ~20-25% in |
| | class III β- | human neural | stability in | hAGOs + ENS; 4 | vitro |
| | tubulin protein | progenitor | neuronal cell | wk in vitro | |
| | | cells | bodies and axons; | hEOs + ENS; 10- | |
| | | | axonal transport | 12 wk in vivo | |
| | | | | hAGOs and | |
| | | | | hFGOs + SM + | |
| | | | | ENS | |
| S100β | Calcium-binding | Pan-glial; | Regulates | 4 wk in vitro | ~5% in vitro |
| | protein B | astrocytes | proliferation and | hAGOs + ENS; | |
| | | | differentiation; | 10-14 wk in vivo | |
| | | | microtubule | hAGOs + ENS | |
| | | | assembly and | | |
| | | | neurite extension; | | |
| | | | calcium efflux | | |
| nNOS | Nitric oxide | Nitrergic | Relaxation of | 4 wk in vitro | ~3% in vitro |
| | synthase | inhibitory | smooth muscle | hAGOs + ENS; | |
| | enzyme | motoneurons | | 10-14 wk in vivo | |
| | | | | hAGOs + ENS | |
| SYP | Synaptophysin, | Interneurons | Neurotransmitter | 4 wk in vitro | N/A |
| | major synaptic | | | hAGOs + ENS | |
| | vesicle protein | | | | |
| CHAT | Choline | Cholinergic | Neurotransmitter | 10 wk in vivo | N/A |
| | acetyltransferase | neurons, | | hAGO + SM + | |
| | | motoneurons | | ENS | |
| TH | Tyrosine | Dopaminergic | Neurotransmitter | 4 wk in vitro | N/A |
| | hydroxylase | peripheral | | hAGOs + ENS; | |
| | enzyme | sympathetic | | 10 wk in vivo | |
| | | neurons | | hAGO + SM + | |
| | | | | ENS | |
| CALB1 | Calbindin 1, | Excitatory | Mediates calcium | 4 wk in vitro | N/A |
| | calcium-binding | motoneurons, | absorption | hAGOs + ENS | |
| | protein | intrinsic | | | |
| | | sensory | | | |
| | | neurons | | | |
| PRPH | Peripherin, type | Peripheral | Neurite | 10-14 wk in vivo | N/A |
| | III intermediate | neurons | elongation and | hAGOs + ENS | |
| | filament | | axonal guidance | | |
| | | | during | | |
| | | | development | | |

### Example 7. ENS Cells Promote the Growth and Gastric Identity of Mesenchyme

Previous studies in developing chicken embryos suggest that ENCCs are involved in gastric mesenchyme development. Therefore, impact of added ENCCs on the development of the small amount of endogenous mesenchyme present in hAGOs was analyzed. Addition of ENCCs at day 6 of hAGO development had little effect on the number of FOXF1+ mesenchyme cells; in contrast, addition of ENCCs at day 9 resulted in 2-4 times more FOXF1+ mesenchyme surrounding the epithelium (FIG. 5A-D). Addition of ENCCs at day 6 or day 9 also correlated with increased levels of gastric mesenchymal genes BARX1, BAPX1, FGF10, ISL1, and SIX2 (FIG. 5E). This suggested that the enteric neurons not only encourage the growth of mesenchyme in vitro, but also support its proper regional patterning into gastric-specific mesenchyme. It is interesting that addition of ENCCs at day 9 promotes the expansion of mesenchyme whereas addition at day 6 does not. The main difference is that ENCCs recombined with hAGOs at day 6 are exposed to the BMP inhibitor NOG and RA from day 6-9 as part of the normal hAGO protocol.

### Example 8. ENCC Cells Support the Growth and Morphogenesis of Organoid Epithelium In Vivo

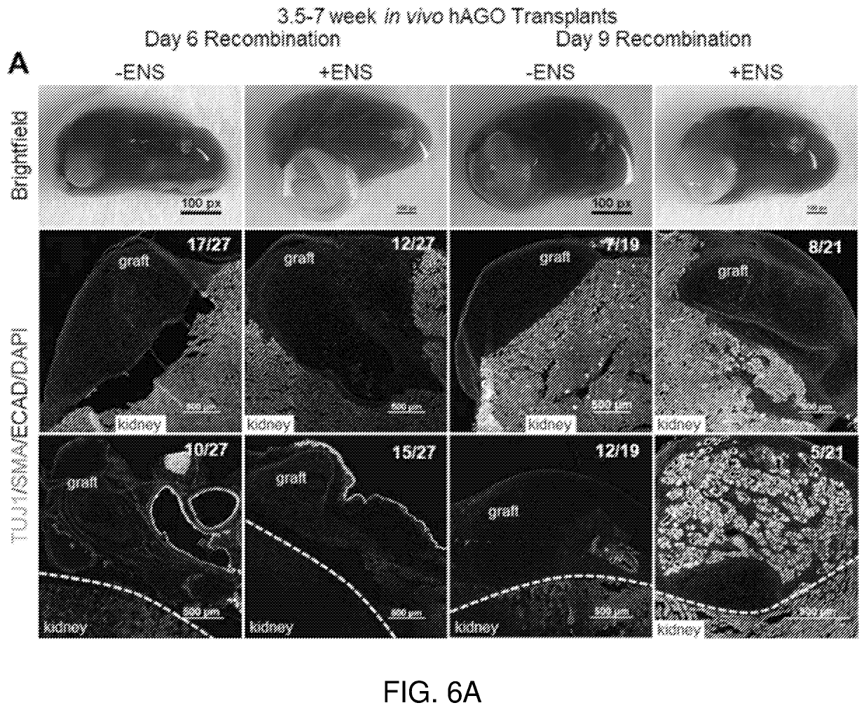

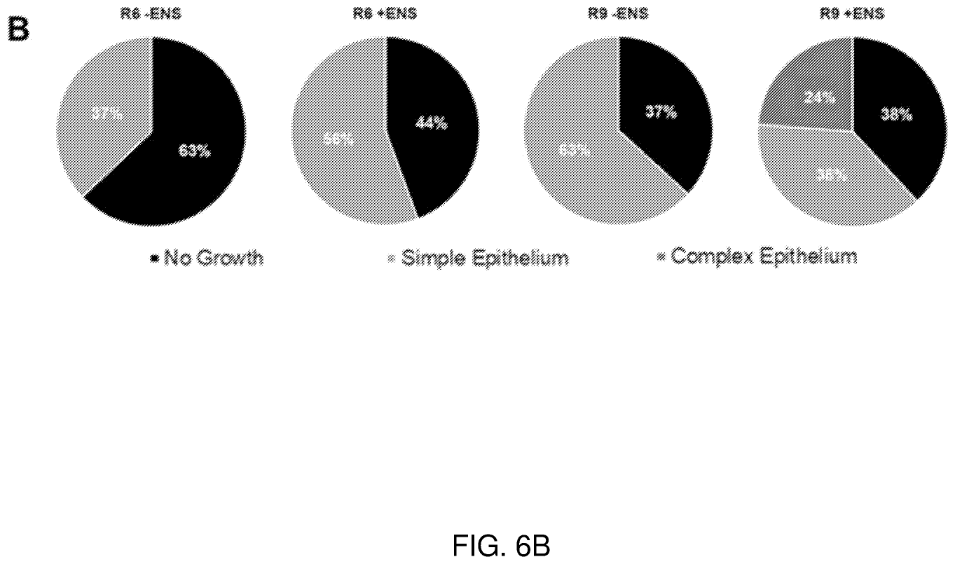

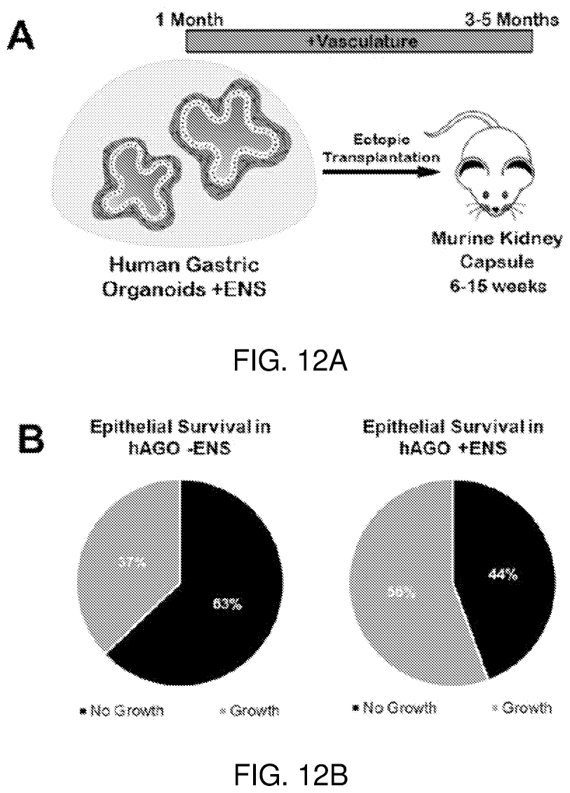

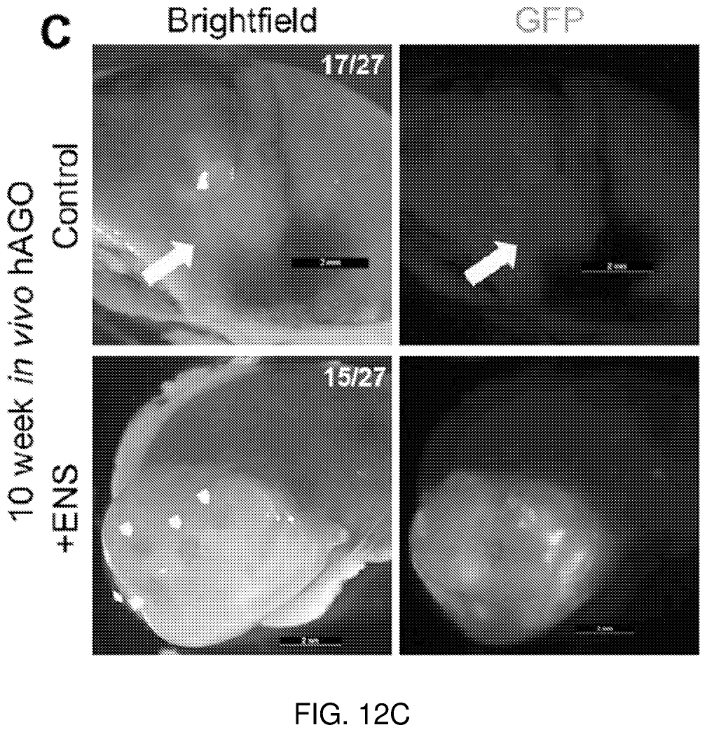

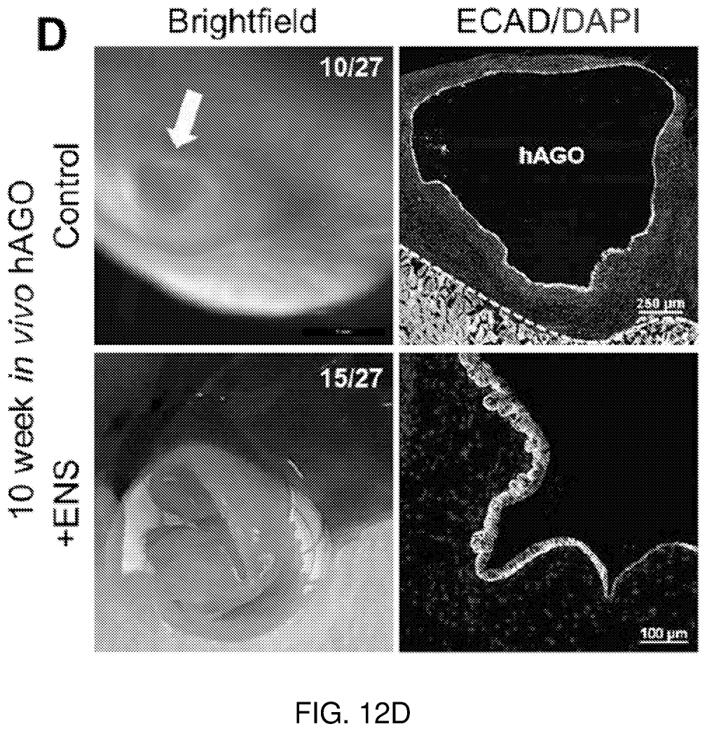

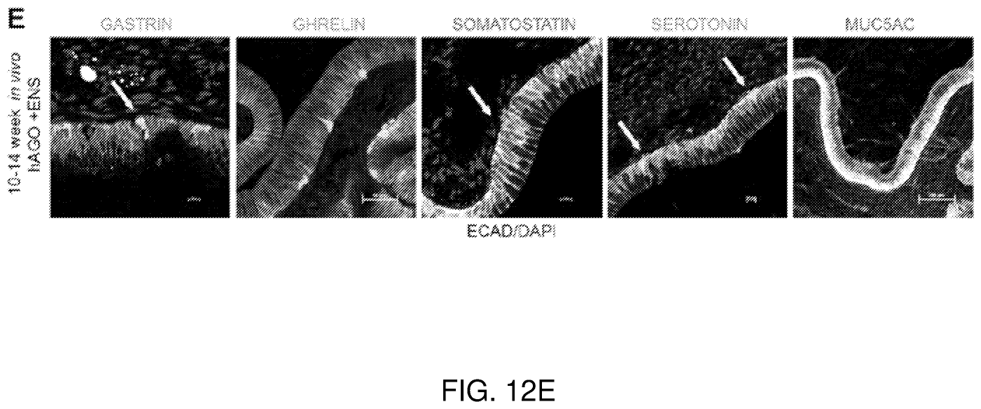

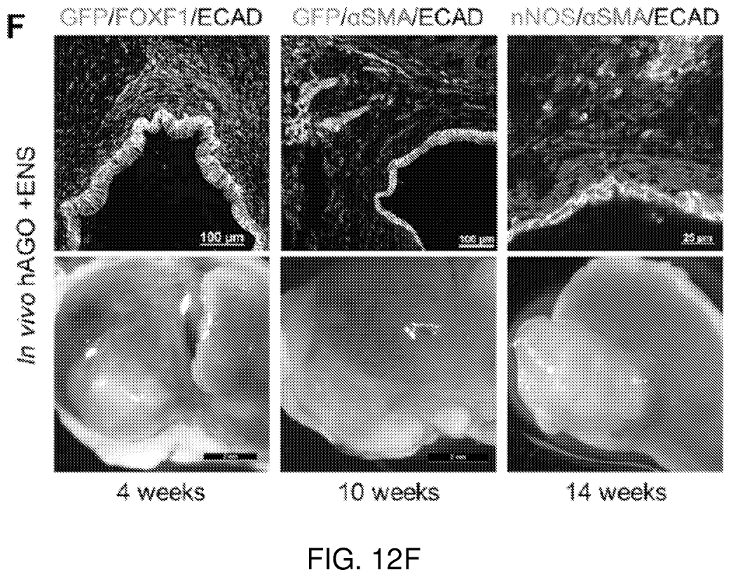

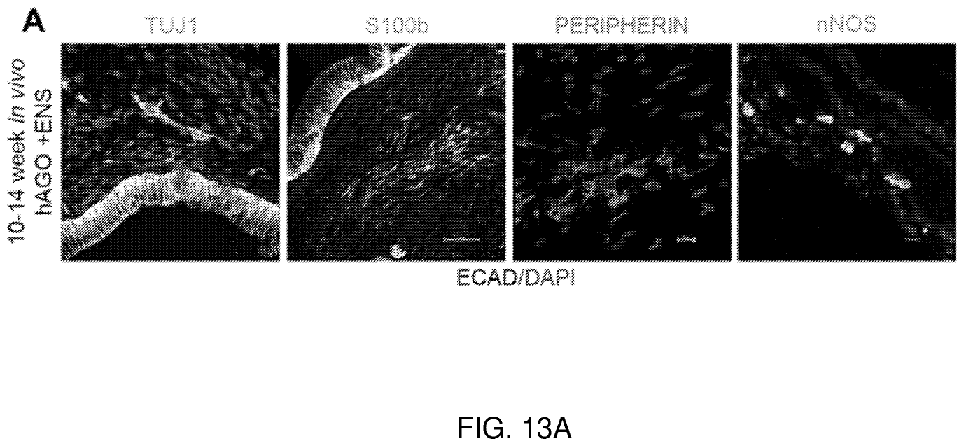

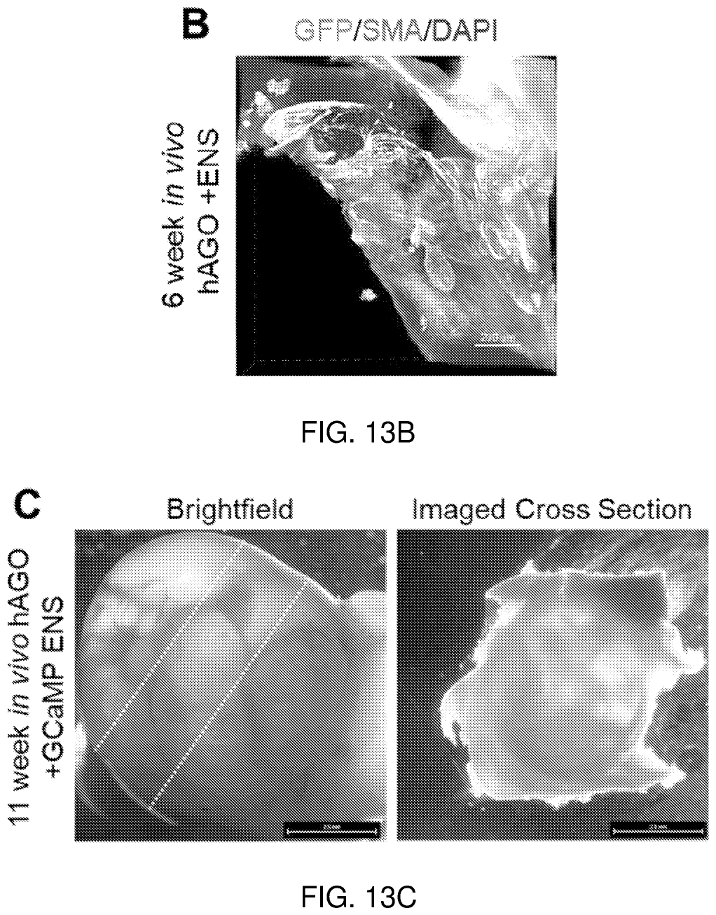

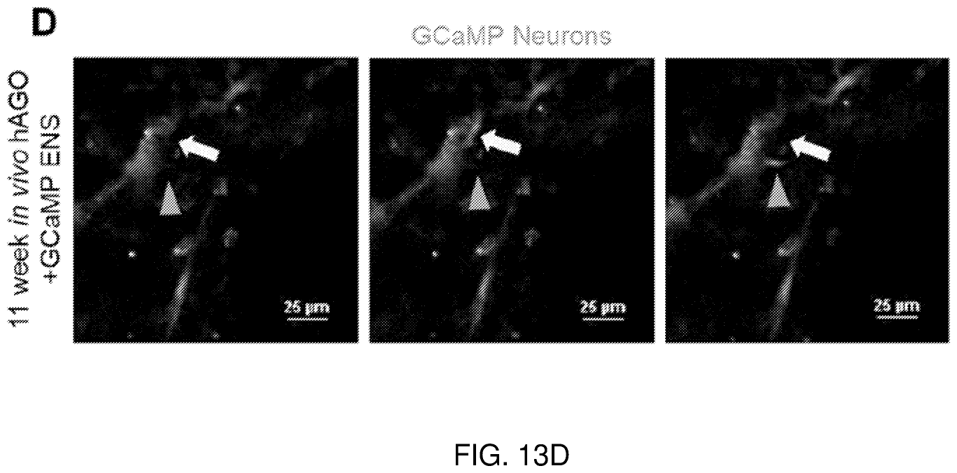

It was shown in FIG. 2 that addition of exogenous mesenchyme alone was not sufficient to promote growth and morphogenesis of organoid epithelium. However, this did not examine how the addition of ENCCs alone without exogenous mesenchyme might impact epithelial development. Therefore, hAGOs with ENCCs recombined at day 6 and 9 were transplanted into mice and grown for 6-15 weeks at which time they were scored for graft survival, overall growth, and epithelial morphogenesis (FIG. 12A). The presence of an ENS, even in the absence of exogenous mesenchyme, improved both number and epithelial growth of hAGO +ENS grafts (FIG. 6A-B, FIG. 12B-D). In most cases, the epithelium of the grafts was a simple gastric epithelium with gastric hormonal cells, such as gastrin, ghrelin, somatostatin, and serotonin, as well as surface mucous cells marked by MUC5AC (FIG. 12E). However, in 5 out of 21 hAGO +ENS grafts, pronounced glandular epithelial morphogenesis was observed as compared to 0 out of 19 hAGO-ENS grafts (FIG. 6A-B). A time course analysis of grafts 4, 10, and 14 weeks following transplantation showed rare examples of differentiated smooth muscle (FIG. 12F) and neurons capable of effluxing calcium as measured using a GCaMP reporter (FIG. 13C-D). Together, these data indicate that ENCCs promote survival and engraftment of hAGOs and the development of glandular tissue in a subset of grafts. However, without a sufficient amount of mesenchyme, addition of ENCCs alone will not result in the development of normal gastric tissue.

### Example 9. The Epithelium of hAGOs +ENCCs is Morphologically and Molecularly Similar to Brunner's Glands

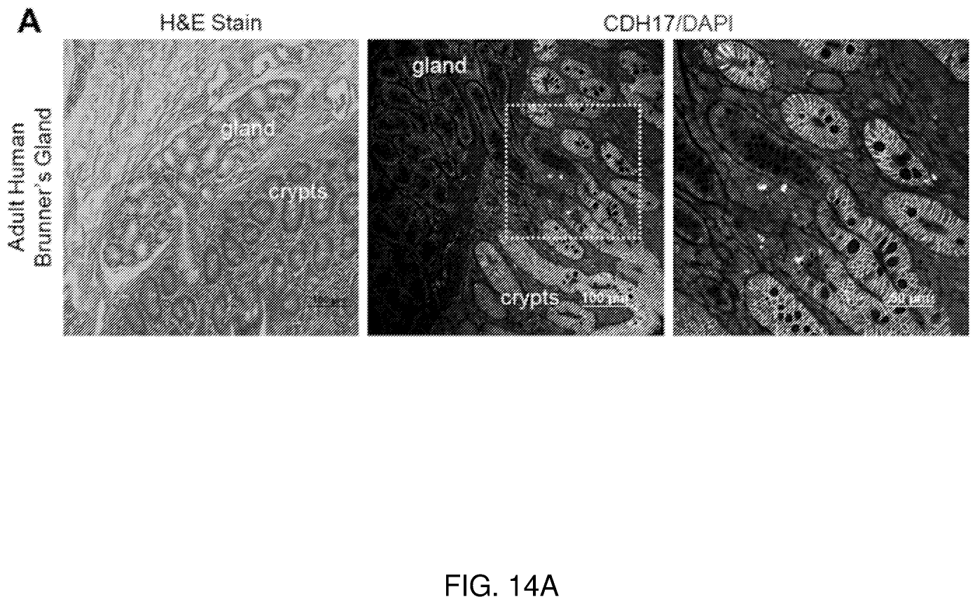

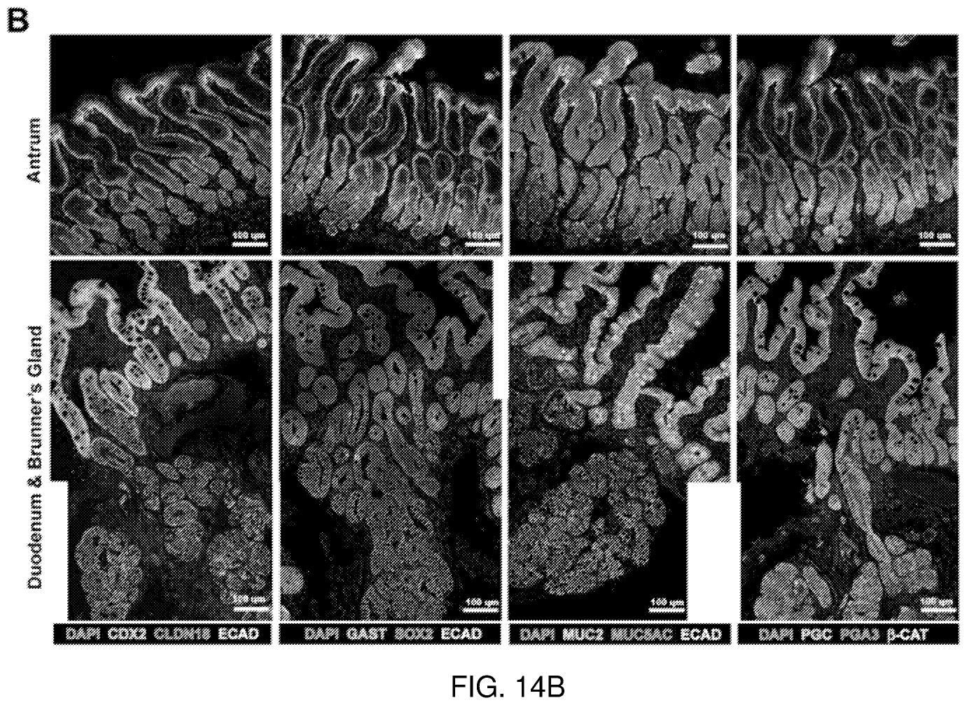

A number of hAGO +ENS grafts displayed a complex glandular epithelial morphology (FIG. 6A-B, FIG. 14A), expressed PDX1 and GATA4 indicative of a gastrointestinal regional identity, and had hormone-expressing cells such as serotonin, ghrelin, histamine, and somatostatin (FIG. 7A). However they did not express key gastric-specific epithelial markers CLDN18 or SOX2 (FIG. 7A-B) or have characteristic gastric cell types MUC5AC-expressing mucous cells (FIG. 7D). The glandular epithelium was also negative for intestinal epithelial markers CDX2 and CDH17 (FIG. 7A-B). It was confirmed that these were human tissue and not a contaminant of mouse tissues from the host. Given that the glandular epithelium of the grafts was neither gastric nor intestinal, the possibility that these were Brunner's glands was explored. Brunner's glands are glandular structures found within the submucosa of the proximal part of the duodenum, near the pyloric junction. They serve to secrete sodium bicarbonate to neutralize any escaping gastric acids. Given the lack of definitive markers for human Brunner's glands, a combinatorial marker profile for Brunner's glands was established using patient biopsies (FIG. 14B) and published reports (FIG. 7C). Human Brunner's glands are negative for gastric markers CLDN18, SOX2, and MUC5AC and intestinal markers CDH17, MUC2 and have low levels of CDX2 compared to adjacent duodenal epithelium (FIG. 14A-B). Human Brunner's glands are positive for glucagon-like peptide-1 receptor (GLP-1R) and MUC6 and co-expression of these markers occurs only in Brunner's glands. The combinatorial expression profile of 9 different markers supports the conclusion that the glandular epithelium of hAGO +ENS grafts is most similar to Brunner's glands (FIG. 7C, FIG. 14B).

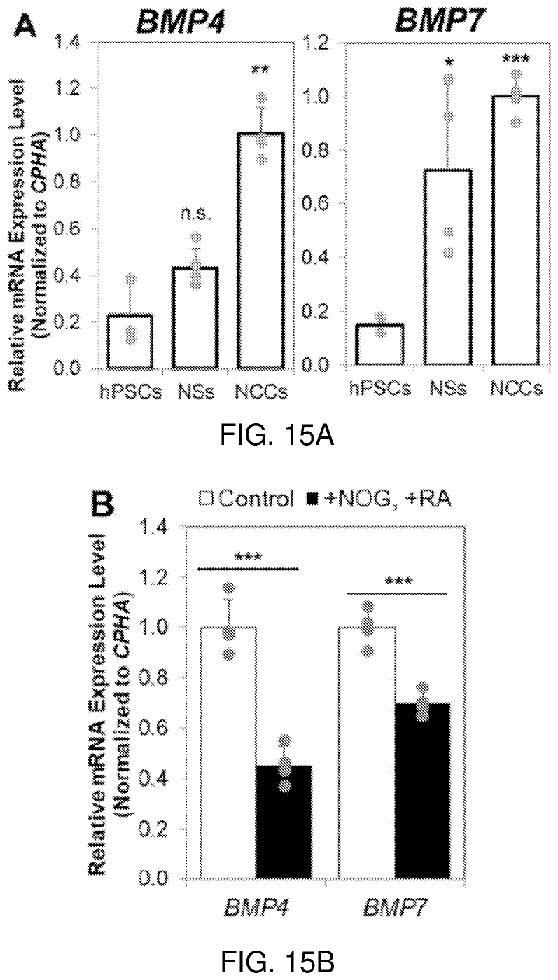

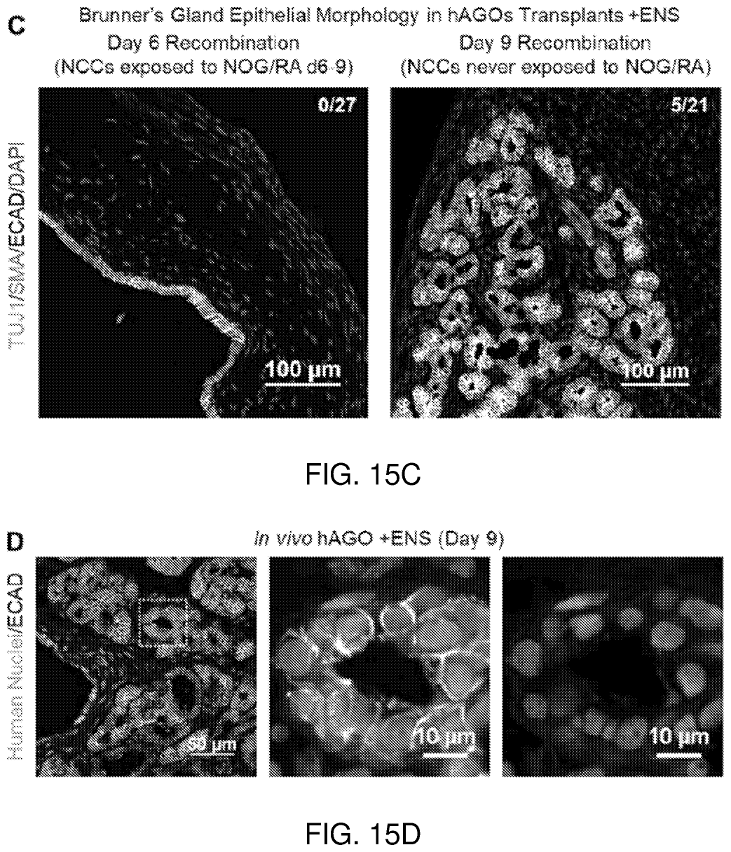

This shift in hAGO epithelium from gastric identity to the more posterior Brunner's gland identity suggest that the added ENCCs were driving this more posterior fate, suggesting that ENCCs, in the absence of mesodermal contribution, may produce a factor(s) that posteriorize gastric epithelium. One candidate pathway was BMP signaling, which is known to promote posterior fate in the gastrointestinal junction. Analysis of ENCCs show high levels of expression of both BMP4 and BMP7 (FIG. 15A). To functionally investigate if BMP activity might mediate the ability of ENCCs to promote a Brunner's gland fate, ENCCs were recombined with hAGO at day 6 and then organoids were cultured with the BMP inhibitor Noggin (NOG) from day 6-9, along with RA, which is a component of the normal hAGO protocol. None of the grafts (0 out of 27) had Brunner's gland epithelium following 3 days of Noggin treatment as compared to hAGOs +ENCCs added at day 9 not treated with NOG (5 out of 21 grafts). To investigate if NOG-treated ENCCs might have reduced posteriorizing activity, ENCC culture were treated with NOG and significantly reduced levels of both BMP4 and 7 were observed (FIG. 15B). Therefore, posteriorizing factors like BMP4 and 7 are produced by ENCCs and that in the presence of BMP inhibitors, ENCC lose their ability to posteriorize gastric epithelium.

### Example 10. Materials and Methods

Animals: All mice used in kidney capsule transplantation experiments were housed in the animal facility at Cincinnati Children's Hospital Medical Center (CCHMC) in accordance with NIH Guidelines for the Care and Use of Laboratory Animals. Animals were maintained on a 12 hour light-dark cycle with access to water and standard chow ad libitum. Healthy male and female immune-deficient NSG (NOD.Cg-Prkdc^{scid}Il2rg^{tm1Wj1}/SzJ) mice, aged between 8 and 16 weeks old, were used in all experiments. These mice were obtained from the Comprehensive Mouse and Cancer Core Facility. All experiments were performed with the approval of the Institutional Animal Care and Use Committee (IACUC) of CCHMC.

Timed matings of wildtype mice were used to generate e13.5 embryos for immunohistological analysis. The morning that the vaginal plug was observed was denoted as e0.5.

Human biopsy tissue: The use of human tissues was approved by an Institutional Review Board (IRB) at CCHMC. Informed consent for the collection and use of tissues was obtained from all donors, parents or legal guardians. Full-thickness fundic and antrum stomach tissue samples obtained from bariatric procedures came from the Helmrath Lab at CCHMC under IRB approval. Human surgical samples were collected from patients between the ages of 15 and 17, and included both males and female of Caucasian and African American backgrounds. Healthy human full-thickness stomach and duodenal tissue samples were obtained from the CCHMC Pathology Core.

Human ESC/iPSC lines and maintenance: Human embryonic stem cell (hESC) lines H1 (WA-01) and H9 (WA-09) were purchased from WiCell. The HI line is male and the H9 line is female. H9-GAPDH-GFP and H9-GAPDH-mCherry hESCs along with human induced pluripotent stem cell (iPSC) line 77.3-GFP were all generated and obtained from the CCHMC Pluripotent Stem Cell Facility (PSCF) and approved by the institutional review board (IRB) at CCHMC. Human iPSC line WTC11 AAVSI-CAG-GCaMP6f was obtained from Bruce Conklin's laboratory at UCSF. All hPSC lines were analyzed for pluripotency and the absence of karyotypic abnormalities and Mycoplasma contamination by the CCHMC PSCF. Human iPSC line WTC11 was analyzed for karyotype by Cell Line Genetics.

All human hPSCs were maintained in an undifferentiated state as colonies in feeder-free conditions. They were plated on human-ES-cell-qualified Matrigel (BD Biosciences) and maintained at 37° C. with 5% CO2 with daily replacement of mTeSR1 media (STEMCELL Technologies). Cells were routinely passaged every 4 days with Dispase (STEMCELL Technologies).

Differentiations of the following lineages for construction of three-germ layer organoids are not dependent or variable based on starting hPSC line.

Differentiation of hPSCs into splanchnic mesenchyme: Partially confluent hPSCs colonies were dissociated into single cells using Accutase (Thermo Fisher Scientific), resuspended in mTesR1 with thiazovivin (1 μM, Tocris) and passaged 1:20 onto new Geltrex-coated 24-well plates (Sigma Aldrich). The directed differentiation of hPSCs into lateral plate mesoderm has been previously described. Briefly, hPSCs were exposed to Activin A (30 ng/ml, Cell Guidance Systems), BMP4 (40 ng/ml, R&D Systems), CHIR99021 (CHIR, 6 μM, ReproCell), FGF2 (20 ng/ml, ThermoFisher Scientific), and PIK90 (100 nM, EMD Millipore) for 24 hours. A basal media composed of Advanced DMEM/F12 (ThermoFisher Scientific) supplemented with B27 supplement (1×, ThermoFisher Scientific), N2 supplement (1×, ThermoFisher Scientific), HEPES (13 mM, ThermoFisher Scientific), L-Glutamine (2 mM ThermoFisher Scientific), and penicillin-streptomycin (1×, ThermoFisher Scientific) was used for this and all subsequent differentiation steps. Cells were then exposed to A8301 (1 μM, Tocris), BMP4 (30 ng/ml), and C59 (1 μM, Cellagen Technology) for 24 hours. For splanchnic mesoderm generation, cells were cultured in A8301 (1 μM), BMP4 (30 ng/ml), C59 (1 μM), FGF2 (20 ng/ml), and RA (2 μM, Sigma-Aldrich) from Day 2 to Day 4. To further direct regional splanchnic mesoderm, RA (2 μM), PMA (2 μM, Tocris) was used for 2 days, and then RA (2 μM), PMA (2 μM), NOG (100 ng/ml, R&D Systems) was used at the last 1 day to promote esophageal/gastric mesenchyme fate. Medium was changed every day throughout protocol. Confluent cells were resuspended using an Accutase treatment (2-3 min) and immediately combined with hAGOs, hFGOs, and hEOs.

Differentiation of hPSCs into ENCCs: The generation of hPSC-derived ENCCs has been previously published. Briefly for ENCC generation, confluent hPSCs were treated with collagenase IV (500 U/ml, Gibco) in mTeSR1 for 60-90 mins to detach colonies. Cells were diluted and washed with DMEM/F-12 (Gibco) and then gently triturated and resuspended in neural induction media, 1:1 ratio of DMEM/F12-GlutaMAX (Gibco) and Neurobasal Medium (Gibco) with B27 supplement (0.5×, Gibco), N2 supplement (0.5×, Gibco), pen-strep (1×, Gibco), insulin (5 μg/mL, Sigma Aldrich), FGF2 (20 ng/mL, R&D Systems), and EGF (20 ng/mL, R&D Systems), on non-TC-treated petri dishes (6 cm, Fisherbrand). Neural induction media was changed daily and all-trans RA (2 μM) was added on days 4 and 5 for posteriorization. Day 6 free-floating neurospheres were plated on human fibronectin (HFN, 3 μg/cm², Corning) and fed neural induction media without RA for 4 days. Migrated cells were collected using a 90 see Accutase treatment and passaged onto HFN. Passaged cells were allowed to grow to confluency for an additional 4 days and fed neural induction media without RA every day. Confluent cells were then collected using a 2-3 min Accutase treatment and immediately combined with hAGOs, hFGOs, and hEOs.

Differentiation of hPSCs into hAGOs, hFGOs, and hEOs: Previously published protocols were slightly modified to generate hAGOs, hFGOs and hEOs. For hAGO and hFGO generation, confluent hPSC cultures were treated with Accutase to resuspend as single cells in mTeSR1 with ROCK inhibitor Y-27632 (10 μM; Tocris) and plated onto a Matrigel-coated 24-well dish (Sigma Aldrich). To direct the differentiation into definitive endoderm (DE), the hPSCs were exposed to Activin A (100 ng/ml) and BMP4 (50 ng/ml) in RPMI 1640 media (Life Technologies). For the following two days, cells were exposed to only Activin A (100 ng/ml) in RPMI 1640 media containing increasing concentrations (0.2% and 2.0%, respectfully) of defined fetal bovine serum (dFBS; HyClone). To then pattern DE into posterior foregut endoderm spheroids, cells were treated with FGF4 (500 ng/ml, R&D systems), NOG (200 ng/ml), and CHIR (2 μM) for 3 days, with media changed daily, in RPMI 1640 with 2% dFBS. RA (2 μM) was added on the third day of FGF4/NOG/CHIR treatment.

Recombination and additional spheroid patterning: Single cell suspensions of mesenchymal cells and ENCCs were counted and added to foregut spheroids at an approximate ratio of 1,000 ENCCs and 2,500 mesenchyme cells per spheroid. Cell mixtures were mixed via gentle pipetting, centrifuged at 300 g for 3-5 minutes, and embedded into 50 μL of basement membrane Matrigel to allow three-dimensional in vitro culture. Organoids were fed with a base media of Advanced DMEM/F12 supplemented with B27 supplement (1×), N2 supplement (1×), HEPES (13 mM), L-Glutamine (2 mM), penicillin-streptomycin (1×), and EGF (100 ng/mL). In addition to this base media, the first three days were supplemented with NOG (200 ng/ml) and RA (2 μM). In addition to EGF, hFGOs were supplemented with CHIR (2 μM) throughout the organoid outgrowth and also received a 48 hr pulse of BMP4 (50 ng/mL) and PD0325901 (2 μM, Stem Cell Technologies) 96 hours prior to collection for parietal cell differentiation in vitro. Media was replaced every 3-4 days. Two weeks following spheroid embedding in Matrigel, the organoids were collected and re-plated in fresh Matrigel at a dilution of ˜1:12.

In vivo transplantation of hAGOs and hFGOs: hAGO, hFGO, hAGO +ENS, hAGO +SM +ENS, hAGO +SM +ENS, and hFGO +SM +ENS were all ectopically transplanted into the kidney capsule of NSG mice. Briefly, four week old hAGOs or hFGOs were removed from Matrigel and transplanted into the kidney subcapsular space. Engrafted organoids were harvested 6-15 weeks after transplantation and analyzed for neuroglial, epithelial, and mesenchymal maturation.

Ex vivo muscle contraction and ENS function: Muscle contraction was assayed as previously described and ENS function and motility were assayed as previously described with slight modifications. Briefly, strips of tissue approximately 2×6 mm in size were dissected and the epithelium mechanically removed in a method similar to seromuscular stripping. No chelation buffer was used. Resulting strips of muscle from hAGO +SM +ENS were mounted within an organ bath chamber system (Radnoti) to isometric force transducers (ADInstruments) and contractile activity continuously recorded using LabChart software (ADInstruments). After an equilibrium period, a logarithmic dose response to Carbamyl-β-methylcholine chloride (Bethanechol; Sigma-Aldrich) was obtained through the administration of exponential doses with concentrations of 1 nM to 10 mM at 2 min intervals before the administration of 10 μM scopolamine (Tocris Bioscience). Data are normalized to muscle strip mass. After another equilibrium period, muscle preparations were then stimulated with a control EFS pulse. NG-nitro-L-arginine methyl ester (L-NAME; 50 μM; Sigma) was applied 10 min before EFS stimulation to observe the effects of NOS inhibition. Without washing, Atropine (atropine sulfate salt monohydrate; 1 μM; Sigma) was the applied 10 min prior to a final EFS stimulation to observe the cumulative effect of NOS and Ach receptor inhibition. After several washes and an additional equilibrium period, another control EFS pulse was administered. Neurotoxin tetrodotoxin (TTX; 4 μM; Tocris) was administered 5 min before a final EFS stimulation. Analysis was performed by calculating the integral (expressed as area under the curve, AUC) immediately before and after stimulation for 60 s. Data are normalized to muscle strip mass.

Ex vivo GCamP6f calcium imaging: Detection of calcium transients was performed using the above-mentioned human iPSC line WTC11 AAVSI-CAG-GCaMP6f. Transplanted hAGOs +ENS were harvested and then cultured on 8-well micro-slide (Ibidi) for 24 hours prior to imaging. They were then imaged every 4-15 sec for 3-10 min using either a 10× or 20× objective on a Nikon Ti-E inverted A1 confocal microscope with NIS elements software to obtain background fluorescence level. Transplanted hAGOs +ENS were then treated with 30 mM KCl. Experiments were carried out at RT.

Tissue processing, immunohistochemistry, and microscopy: Cell monolayers, ENCCs, and day 0 spheroids were washed with 1× phosphate-buffered saline (PBS), fixed with 4% paraformaldehyde (PFA) at room temperature (RT) for 15 min, washed, and stored in PBS at 4° C. Four week old in vitro organoids and in vivo transplants were washed with PBS, fixed in 4% PFA at 4° C. overnight, washed, and then placed in either PBS, 30% sucrose in PBS, or 70% ethanol at 4° C. overnight for downstream whole mount, cryogenic, or paraffin processing, respectively. Prior to fixation, whole mount tissues were extracted from Matrigel using manual pipetting in cold PBS and Cell Recovery Solution (Corning). Tissues were then embedded in either O.C.T. Compound (Tissue-Tek) or paraffin and were serially sectioned at a thickness of 7-8 μm onto Superfrost Plus glass slides (Fisherbrand). Cryosection slides and paraffin slides were stored at −80° C. and RT, respectively. Routine Hematoxylin & Eosin (H&E) staining was performed by the Research Pathology Core at CCHMC.

Frozen slides were thawed to room temperature (RT) and rehydrated in PBS, while paraffin slides were deparaffinized, rehydrated, and subjected to heat- and pressure-induced antigen retrieval in citrate buffer (0.192% citric acid and 0.0005% Tween-20 in dH₂0 of pH 6.0 with NaOH) for 30 minutes and brought to RT on ice. All slides and cells were washed with PBS, permeabilized with 0.5% Triton X-100 in PBS (PBST) for 15 min at RT and then blocked with 5% normal donkey serum (NDS, Jackson ImmunoResearch) in PBS for one hour at RT. Tissue was incubated at 4° C. overnight in primary antibodies diluted in 5% NDS in PBS. The following day, tissue was washed and incubated with secondary antibodies at RT for one hour, thoroughly washed, and cover slipped with Fluoromount-G (Southern Biotech).

For wholemount staining, organoids were washed at RT and then permeabilized with PBST at 4° C. overnight. The next day, organoids were blocked in 5% NDS in PBST for 6-8 hours at RT and then incubated in primary antibodies at 4° C. overnight on a rocking platform. Organoids were extensively washed in PBST and then incubated in secondary antibodies at 4° C. overnight. Finally, organoids were washed with PBST, PBS and then serially dehydrated to 100% methanol. Organoids were then optically cleared with Murray's Clear (2:1 benzyl benzoate:benzyl alcohol, Sigma) for at least 15 minutes prior to imaging.

Brightfield and GFP fluorescence images of live tissue samples were captured using either a Leica DMC5400 or DFC310 FX camera attached to a stereomicroscope. Whole mount and all immunofluorescent images were captured using a Nikon Ti-E inverted A1 confocal microscope. Images were processed and quantified using Nikon NIS Elements, Bitplane Imaris, Adobe Illustrator, and Microsoft PowerPoint software.

RNA isolation and quantitative real-time PCR (qRT-PCR): Spheroids and organoids were harvested in RAI Lysis Buffer and β-mercapethanol and stored at −80° C. until total RNA was isolated using NucleoSpin RNA Isolation Kit (Macherey-Nagel) according to manufacturers' instructions. Complementary DNA (cDNA) was reverse transcribed from 116 ng of RNA using a SuperScript VILO cDNA Synthesis Kit (Invitrogen). qRT-PCR was performed using a QuantiTect SYBR Green PCR Kit (Qiagen) in MicroAmp EnduraPlate Optical 96-Well Fast Reaction Plates (Applied Biosystems) and run on a QuantStudio 6 Real-Time PCR Detection System (Applied Biosystems). Analysis was performed using the ΔΔCt method by first normalizing all cycle threshold (Ct) values to a base housekeeping gene (GAPDH, PPIA, or FOXF1) and then to the control hAGO samples. Statistical analysis was performed using Student's 1-test.

Statistical analyses: For analysis of organoid patterning, “n” represents the number of replicates performed in each experiment and each replicate is defined as 1 well of approx. 3-5 organoids in Matrigel culture. All data are represented as mean±s.d. Student's t-tests with 2-tailed distribution and un-equal variance was completed using Microsoft Excel, where p≤0.05 is symbolized by *, p≤0.01 is symbolized by **, and p≤0.001 is symbolized by ***. The determined significance cutoff was p≤0.05. No statistical method was used to predetermine sample size. The investigators were not blinded to allocation during experiments and outcome assessment. No randomization was made.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Bajpai, R., Chen, D. A., Rada-Iglesias, A., Zhang, J., Xiong, Y., Helms, J., Chang, C. P., Zhao, Y., Swigut, T. and Wysocka, J. (2010) ‘CHD7 cooperates with PBAF to control multipotent neural crest formation’, Nature, 463(7283), pp. 958-62.
Baker, C., Ahmed, M., Cheng, K., Arciero, E., Bhave, S., Ho, W. L. N., Goldstein, A. M. and Hotta, R. (2020) ‘Hypoganglionosis in the gastric antrum causes delayed gastric emptying’, Neurogastroenterol Motil, 32(5), pp. e13766.
Balbinot, C., Vanier, M., Armant, O., Nair, A., Penichon, J., Soret, C., Martin, E., Saandi, T., Reimund, J. M., Deschamps, J., Beck, F., Domon-Dell, C., Gross, I., Duluc, I. and Freund, J. N. (2017) ‘Fine-tuning and autoregulation of the intestinal determinant and tumor suppressor homeobox gene CDX2 by alternative splicing’, Cell Death Differ, 24(12), pp. 2173-2186.
Barber, K., Studer, L. and Fattahi, F. (2019) ‘Derivation of enteric neuron lineages from human pluripotent stem cells’, Nat Protoc, 14(4), pp. 1261-1279.
Beckett, E. A., Sanders, K. M. and Ward, S. M. (2017) ‘Inhibitory responses mediated by vagal nerve stimulation are diminished in stomachs of mice with reduced intramuscular interstitial cells of Cajal’, Sci Rep, 7, pp. 44759.
Bohorquez, D. V., Shahid, R. A., Erdmann, A., Kreger, A. M., Wang, Y., Calakos, N., Wang, F. and Liddle, R. A. (2015) ‘Neuroepithelial circuit formed by innervation of sensory enteroendocrine cells’, J Clin Invest, 125(2), pp. 782-6.
Breit, S., Kupferberg, A., Rogler, G. and Hasler, G. (2018) ‘Vagus Nerve as Modulator of the Brain-Gut Axis in Psychiatric and Inflammatory Disorders’, Front Psychiatry, 9, pp. 44.
Brookes, S. J., Spencer, N. J., Costa, M. and Zagorodnyuk, V. P. (2013) ‘Extrinsic primary afferent signalling in the gut’, Nat Rev Gastroenterol Hepatol, 10(5), pp. 286-96.
Chen, T. W., Wardill, T. J., Sun, Y., Pulver, S. R., Renninger, S. L., Baohan, A., Schreiter, E. R., Kerr, R. A., Orger, M. B., Jayaraman, V., Looger, L. L., Svoboda, K. and Kim, D. S. (2013) ‘Ultrasensitive fluorescent proteins for imaging neuronal activity’, Nature, 499(7458), pp. 295-300.
Choi, E., Roland, J. T., Barlow, B. J., O'Neal, R., Rich, A. E., Nam, K. T., Shi, C. and Goldenring, J. R. (2014) ‘Cell lineage distribution atlas of the human stomach reveals heterogeneous gland populations in the gastric antrum’, Gut, 63(11), pp. 1711-20.
Davenport, C., Diekmann, U., Budde, I., Detering, N. and Naujok, O. (2016) ‘Anterior-Posterior Patterning of Definitive Endoderm Generated from Human Embryonic Stem Cells Depends on the Differential Signaling of Retinoic Acid, Wnt-, and BMP-Signaling’, Stem Cells, 34(11), pp. 2635-2647.
de Santa Barbara, P., van den Brink, G. R. and Roberts, D. J. (2002) ‘Molecular etiology of gut malformations and diseases’, Am J Med Genet, 115(4), pp. 221-30.
De Santa Barbara, P., Williams, J., Goldstein, A. M., Doyle, A. M., Nielsen, C., Winfield,
S., Faure, S. and Roberts, D. J. (2005) ‘Bone morphogenetic protein signaling pathway plays multiple roles during gastrointestinal tract development’, Dev Dyn, 234(2), pp. 312-22.
Eicher, A. K., Berns, H. M. and Wells, J. M. (2018) ‘Translating Developmental Principles to Generate Human Gastric Organoids’, Cell Mol Gastroenterol Hepatol, 5(3), pp. 353-363.
Faure, S., de Santa Barbara, P., Roberts, D. J. and Whitman, M. (2002) ‘Endogenous patterns of BMP signaling during early chick development’, Dev Biol, 244(1), pp. 44-65.
Faure, S., Georges, M., McKey, J., Sagnol, S. and de Santa Barbara, P. (2013) ‘Expression pattern of the homeotic gene Bapx1 during early chick gastrointestinal tract development’, Gene Expr Patterns, 13(8), pp. 287-92.
Faure, S., McKey, J., Sagnol, S. and de Santa Barbara, P. (2015) ‘Enteric neural crest cells regulate vertebrate stomach patterning and differentiation’, Development, 142(2), pp. 331-42.
Freddo, A. M., Shoffner, S. K., Shao, Y., Taniguchi, K., Grosse, A. S., Guysinger, M. N., Wang, S., Rudraraju, S., Margolis, B., Garikipati, K., Schnell, S. and Gumucio, D. L. (2016) ‘Coordination of signaling and tissue mechanics during morphogenesis of murine intestinal villi: a role for mitotic cell rounding’, Integr Biol (Camb), 8(9), pp. 918-28.
Furness, J. B., Di Natale, M., Hunne, B., Oparija-Rogenmozere, L., Ward, S. M., Sasse, K. C., Powley, T. L., Stebbing, M. J., Jaffey, D. and Fothergill, L. J. (2020) ‘The identification of neuronal control pathways supplying effector tissues in the stomach’, Cell Tissue Res, 382(3), pp. 433-445.
Gilbert, M. A., Schultz-Rogers, L., Rajagopalan, R., Grochowski, C. M., Wilkins, B. J., Biswas, S., Conlin, L. K., Fiorino, K. N., Dhamija, R., Pack, M. A., Klee, E. W., Piccoli, D. A. and Spinner, N. B. (2020) ‘Protein-elongating mutations in MYH11 are implicated in a dominantly inherited smooth muscle dysmotility syndrome with severe esophageal, gastric, and intestinal disease’, Hum Mutat, 41(5), pp. 973-982.
Goodwin, K., Mao, S., Guyomar, T., Miller, E., Radisky, D. C., Košmrlj, A. and Nelson, C. M. (2019) ‘Smooth muscle differentiation shapes domain branches during mouse lung development’, Development, 146(22).
Han, L., Chaturvedi, P., Kishimoto, K., Koike, H., Nasr, T., Iwasawa, K., Giesbrecht, K., Witcher, P. C., Eicher, A., Haines, L., Lee, Y., Shannon, J. M., Morimoto, M., Wells, J. M., Takebe, T. and Zorn, A. M. (2020) ‘Single cell transcriptomics identifies a signaling network coordinating endoderm and mesoderm diversification during foregut organogenesis’, Nature Communications, 11.
Huycke, T. R., Miller, B. M., Gill, H. K., Nerurkar, N. L., Sprinzak, D., Mahadevan, L. and Tabin, C. J. (2019) ‘Genetic and Mechanical Regulation of Intestinal Smooth Muscle Development’, Cell, 179(1), pp. 90-105.e21.
Iino, S. and Horiguchi, K. (2006) ‘Interstitial cells of cajal are involved in neurotransmission in the gastrointestinal tract’, Acta Histochem Cytochem, 39(6), pp. 145-53.
Kaelberer, M. M., Buchanan, K. L., Klein, M. E., Barth, B. B., Montoya, M. M., Shen, X. and Bohorquez, D. V. (2018) ‘A gut-brain neural circuit for nutrient sensory transduction’, Science, 361(6408).
Kim, B. M., Buchner, G., Miletich, I., Sharpe, P. T. and Shivdasani, R. A. (2005) ‘The stomach mesenchymal transcription factor Barx1 specifies gastric epithelial identity through inhibition of transient Wnt signaling’, Dev Cell, 8(4), pp. 611-22.
Kim, T. H. and Shivdasani, R. A. (2016) ‘Stomach development, stem cells and disease’, Development, 143(4), pp. 554-65.
Knox, S. M., Lombaert, I. M., Reed, X., Vitale-Cross, L., Gutkind, J. S. and Hoffman, M. P. (2010) ‘Parasympathetic innervation maintains epithelial progenitor cells during salivary organogenesis’, Science, 329(5999), pp. 1645-7.
Lasrado, R., Boesmans, W., Kleinjung, J., Pin, C., Bell, D., Bhaw, L., McCallum, S., Zong, H., Luo, L., Clevers, H., Vanden Berghe, P. and Pachnis, V. (2017) ‘Lineage-dependent spatial and functional organization of the mammalian enteric nervous system’, Science, 356(6339), pp. 722-726.
Le Guen, L., Marchal, S., Faure, S. and de Santa Barbara, P. (2015) ‘Mesenchymal-epithelial interactions during digestive tract development and epithelial stem cell regeneration’, Cell Mol Life Sci, 72(20), pp. 3883-96.
Li, Z., Chalazonitis, A., Huang, Y. Y., Mann, J. J., Margolis, K. G., Yang, Q. M., Kim, D. O., Côté, F., Mallet, J. and Gershon, M. D. (2011) ‘Essential roles of enteric neuronal serotonin in gastrointestinal motility and the development/survival of enteric dopaminergic neurons’, J Neurosci, 31(24), pp. 8998-9009.
Loh, K. M., Chen, A., Koh, P. W., Deng, T. Z., Sinha, R., Tsai, J. M., Barkal, A. A., Shen, K. Y., Jain, R., Morganti, R. M., Shyh-Chang, N., Fernhoff, N. B., George, B. M., Wernig, G., Salomon, R. E. A., Chen, Z., Vogel, H., Epstein, J. A., Kundaje, A., Talbot, W. S., Beachy, P. A., Ang, L. T. and Weissman, I. L. (2016) ‘Mapping the Pairwise Choices Leading from Pluripotency to Human Bone, Heart, and Other Mesoderm Cell Types’, Cell, 166(2), pp. 451-467.
McCauley, H. A., Matthis, A. L., Enriquez, J. R., Nichol, J. T., Sanchez, J. G., Stone, W. J., Sundaram, N., Helmrath, M. A., Montrose, M. H., Aihara, E. and Wells, J. M. (2020) ‘Enteroendocrine cells couple nutrient sensing to nutrient absorption by regulating ion transport’, Nat Commun, 11(1), pp. 4791.
McCracken, K. W., Aihara, E., Martin, B., Crawford, C. M., Broda, T., Treguier, J., Zhang, X., Shannon, J. M., Montrose, M. H. and Wells, J. M. (2017) ‘Wnt/beta-catenin promotes gastric fundus specification in mice and humans’, Nature, 541(7636), pp. 182-187.
McCracken, K. W., Cata, E. M., Crawford, C. M., Sinagoga, K. L., Schumacher, M., Rockich, B. E., Tsai, Y. H., Mayhew, C. N., Spence, J. R., Zavros, Y. and Wells, J. M. (2014) ‘Modelling human development and disease in pluripotent stem-cell-derived gastric organoids’, Nature, 516(7531), pp. 400-4.
Moniot, B., Biau, S., Faure, S., Nielsen, C. M., Berta, P., Roberts, D. J. and de Santa Barbara, P. (2004) ‘SOX9 specifies the pyloric sphincter epithelium through mesenchymal-epithelial signals’, Development, 131(15), pp. 3795-804.
Munera, J. O. and Wells, J. M. (2017) ‘Generation of Gastrointestinal Organoids from Human Pluripotent Stem Cells’, Methods Mol Biol, 1597, pp. 167-177.
Múnera, J. O., Sundaram, N., Rankin, S. A., Hill, D., Watson, C., Mahe, M., Vallance, J. E., Shroyer, N. F., Sinagoga, K. L., Zarzoso-Lacoste, A., Hudson, J. R., Howell, J. C., Chatuvedi, P., Spence, J. R., Shannon, J. M., Zorn, A. M., Helmrath, M. A. and Wells, J. M. (2017) ‘Differentiation of Human Pluripotent Stem Cells into Colonic Organoids via Transient Activation of BMP Signaling’, Cell Stem Cell, 21(1), pp. 51-64.e6.
Nagy, N., Barad, C., Graham, H. K., Hotta, R., Cheng, L. S., Fejszak, N. and Goldstein, A. M. (2016) ‘Sonic hedgehog controls enteric nervous system development by patterning the extracellular matrix’, Development, 143(2), pp. 264-75.
Nagy, N. and Goldstein, A. M. (2017) ‘Enteric nervous system development: A crest cell's journey from neural tube to colon’, Semin Cell Dev Biol, 66, pp. 94-106.
Nedvetsky, P. I., Emmerson, E., Finley, J. K., Ettinger, A., Cruz-Pacheco, N., Prochazka, J., Haddox, C. L., Northrup, E., Hodges, C., Mostov, K. E., Hoffman, M. P. and Knox, S. M. (2014) ‘Parasympathetic innervation regulates tubulogenesis in the developing salivary gland’, Dev Cell, 30(4), pp. 449-62.
Norlen, P., Ericsson, P., Kitano, M., Ekelund, M. and Hakanson, R. (2005) ‘The vagus regulates histamine mobilization from rat stomach ECL cells by controlling their sensitivity to gastrin’, J Physiol, 564(Pt 3), pp. 895-905.
Poling, H. M., Wu, D., Brown, N., Baker, M., Hausfeld, T. A., Huynh, N., Chaffron, S., Dunn, J. C. Y., Hogan, S. P., Wells, J. M., Helmrath, M. A. and Mahe, M. M. (2018) ‘Mechanically induced development and maturation of human intestinal organoids in vivo’, Nat Biomed Eng, 2(6), pp. 429-442.
Rakhilin, N., Barth, B., Choi, J., Munoz, N. L., Kulkarni, S., Jones, J. S., Small, D. M., Cheng, Y. T., Cao, Y., La Vinka, C., Kan, E., Dong, X., Spencer, M., Pasricha, P., Nishimura, N. and Shen, X. (2016) ‘Simultaneous optical and electrical in vivo analysis of the enteric nervous system’, Nat Commun, 7, pp. 11800.
Roberts, D. J., Johnson, R. L., Burke, A. C., Nelson, C. E., Morgan, B. A. and Tabin, C. (1995) ‘Sonic hedgehog is an endodermal signal inducing Bmp-4 and Hox genes during induction and regionalization of the chick hindgut’, Development, 121(10), pp. 3163-74.
Roberts, D. J., Smith, D. M., Goff, D. J. and Tabin, C. J. (1998) ‘Epithelial-mesenchymal signaling during the regionalization of the chick gut’, Development, 125(15), pp. 2791-801.
Rydning, A., Lyng, O., Falkmer, S. and Grønbech, J. E. (2002) ‘Histamine is involved in gastric vasodilation during acid back diffusion via activation of sensory neurons’, Am J Physiol Gastrointest Liver Physiol, 283(3), pp. G603-11.
Shaylor, L. A., Hwang, S. J., Sanders, K. M. and Ward, S. M. (2016) ‘Convergence of inhibitory neural inputs regulate motor activity in the murine and monkey stomach’, Am J Physiol Gastrointest Liver Physiol, 311(5), pp. G838-g851.
Shyer, A. E., Tallinen, T., Nerurkar, N. L., Wei, Z., Gil, E. S., Kaplan, D. L., Tabin, C. J. and Mahadevan, L. (2013) ‘Villification: how the gut gets its villi’, Science, 342(6155), pp. 212-8.
Smith, D. M., Nielsen, C., Tabin, C. J. and Roberts, D. J. (2000) ‘Roles of BMP signaling and Nkx2.5 in patterning at the chick midgut-foregut boundary’, Development, 127(17), pp. 3671-81.
Smith, D. M. and Tabin, C. J. (1999) ‘BMP signalling specifies the pyloric sphincter’, Nature, 402(6763), pp. 748-9.
Stevens, M. L., Chaturvedi, P., Rankin, S. A., Macdonald, M., Jagannathan, S., Yukawa, M., Barski, A. and Zorn, A. M. (2017) ‘Genomic integration of Wnt/beta-catenin and BMP/Smad1 signaling coordinates foregut and hindgut transcriptional programs’, Development, 144(7), pp. 1283-1295.
Sung, T. S., Hwang, S. J., Koh, S. D., Bayguinov, Y., Peri, L. E., Blair, P. J., Webb, T. I., Pardo, D. M., Rock, J. R., Sanders, K. M. and Ward, S. M. (2018) ‘The cells and conductance mediating cholinergic neurotransmission in the murine proximal stomach’, J Physiol, 596(9), pp. 1549-1574.
Tan, S. H., Swathi, Y., Tan, S., Goh, J., Seishima, R., Murakami, K., Oshima, M., Tsuji, T., Phuah, P., Tan, L. T., Wong, E., Fatehullah, A., Sheng, T., Ho, S. W. T., Grabsch, H. I., Srivastava, S., Teh, M., Denil, S., Mustafah, S., Tan, P., Shabbir, A., So, J., Yeoh, K. G. and Barker, N. (2020) ‘AQP5 enriches for stem cells and cancer origins in the distal stomach’, Nature, 578(7795), pp. 437-443.
Theodosiou, N. A. and Tabin, C. J. (2005) ‘Sox9 and Nkx2.5 determine the pyloric sphincter epithelium under the control of BMP signaling’, Dev Biol, 279(2), pp. 481-90.
Tiso, N., Filippi, A., Pauls, S., Bortolussi, M. and Argenton, F. (2002) ‘BMP signalling regulates anteroposterior endoderm patterning in zebrafish’, Mech Dev, 118(1-2), pp. 29-37.
Walsh, K. T. and Zemper, A. E. (2019) ‘The Enteric Nervous System for Epithelial Researchers: Basic Anatomy, Techniques, and Interactions With the Epithelium’, Cell Mol Gastroenterol Hepatol, 8(3), pp. 369-378.
Walton, K. D., Kolterud, A., Czerwinski, M. J., Bell, M. J., Prakash, A., Kushwaha, J., Grosse, A. S., Schnell, S. and Gumucio, D. L. (2012) ‘Hedgehog-responsive mesenchymal clusters direct patterning and emergence of intestinal villi’, Proc Natl Acad Sci USA, 109(39), pp. 15817-22.
Wang, Y., Shi, C., Lu, Y., Poulin, E. J., Franklin, J. L. and Coffey, R. J. (2015) ‘Loss of Lrig1 leads to expansion of Brunner glands followed by duodenal adenomas with gastric metaplasia’, Am J Pathol, 185(4), pp. 1123-34.
Ward, S. M. and Sanders, K. M. (2006) ‘Involvement of intramuscular interstitial cells of Cajal in neuroeffector transmission in the gastrointestinal tract’, J Physiol, 576(Pt 3), pp. 675-82.
Watson, C. L., Mahe, M. M., Munera, J., Howell, J. C., Sundaram, N., Poling, H. M., Schweitzer, J. I., Vallance, J. E., Mayhew, C. N., Sun, Y., Grabowski, G., Finkbeiner, S. R., Spence, J. R., Shroyer, N. F., Wells, J. M. and Helmrath, M. A. (2014) ‘An in vivo model of human small intestine using pluripotent stem cells’, Nat Med, 20(11), pp. 1310-4.
Westfal, M. L. and Goldstein, A. M. (2017) ‘Pediatric enteric neuropathies: diagnosis and current management’, Curr Opin Pediatr.
Workman, M. J., Mahe, M. M., Trisno, S., Poling, H. M., Watson, C. L., Sundaram, N., Chang, C. F., Schiesser, J., Aubert, P., Stanley, E. G., Elefanty, A. G., Miyaoka, Y., Mandegar, M. A., Conklin, B. R., Neunlist, M., Brugmann, S. A., Helmrath, M. A. and Wells, J. M. (2017) ‘Engineered human pluripotent-stem-cell-derived intestinal tissues with a functional enteric nervous system’, Nat Med, 23(1), pp. 49-59.
Zhang, X., McGrath, P. S., Salomone, J., Rahal, M., McCauley, H. A., Schweitzer, J., Kovall, R., Gebelein, B. and Wells, J. M. (2019) ‘A Comprehensive Structure-Function Study of Neurogenin3 Disease-Causing Alleles during Human Pancreas and Intestinal Organoid Development’, Dev Cell, 50(3), pp. 367-380.e7.
Zhao, C. M., Martinez, V., Piqueras, L., Wang, L., Taché, Y. and Chen, D. (2008) ‘Control of gastric acid secretion in somatostatin receptor 2 deficient mice: shift from endocrine/paracrine to neurocrine pathways’, Endocrinology, 149(2), pp. 498-505.

1. A method for preparing a gastrointestinal organoid from the three primary germ layers, comprising:
contacting gut endoderm spheroids with splanchnic mesoderm cells (SM) and enteric neural crest cells (ENCCs) to form a cell mixture; and
culturing the cell mixture under conditions sufficient to differentiate the cell mixture into a gastrointestinal organoid comprising epithelium, mesenchyme, and a functional enteric nervous system (ENS).

2. The method of claim 1, wherein one or more of the gut endoderm spheroids, the SM, or the ENCCs have been derived from pluripotent stem cells.

3. The method of claim 1, wherein the gut endoderm spheroids have been derived from definitive endoderm cells; and/or wherein the gut endoderm spheroids are spontaneously formed gut endoderm spheroids that develop during differentiation of definitive endoderm cells into gut endoderm.

4. The method of claim 3, wherein the definitive endoderm cells have been derived from pluripotent stem cells.

5. (canceled)

6. The method of claim 1, wherein the SM and ENCCs are not contacted with a suspension of single gut endoderm cells or aggregated gut endoderm spheroids that are produced by aggregating a suspension of single gut endoderm cells, and/or wherein the gut endoderm spheroids and ENCCs are not contacted with cardiac mesenchyme, septum transversum, or gastric/esophageal mesenchyme cells.

7. (canceled)

8. The method of claim 1, wherein the SM have been derived from pluripotent stem cells according to a method comprising:
a) activating a TGF-b pathway, a Wnt pathway, an FGF pathway, and a BMP pathway, and inhibiting a PI3K pathway in the pluripotent stem cells for a first period to differentiate the pluripotent stem cells to middle primitive streak cells;
b) activating a TGF-b pathway, a Wnt pathway, an FGF pathway, and a BMP pathway in the middle primitive streak cells for a second period to differentiate the middle primitive streak cells to lateral plate mesoderm cells; and
c) activating a TGF-b pathway, a Wnt pathway, an FGF pathway, and a BMP pathway in the lateral plate mesoderm cells, in combination with retinoic acid, for a third period to differentiate the lateral plate mesoderm cells to SM; and/or
wherein the ENCCs have been derived from pluripotent stem cells according to a method comprising:
a) activating an FGF pathway and an EGF pathway in the pluripotent stem cells for a fourth period, and activating an FGF pathway and an EGF pathway in combination with retinoic acid for a fifth period, to differentiate the pluripotent stem cells to neurospheres comprising the ENCCs;
b) culturing the neurospheres on an extracellular matrix under conditions to allow the ENCCs to migrate from the neurospheres as single cells; and
c) collecting the ENCCs that have migrated from the neurospheres as the single cells,
thereby producing the ENCCs.

9. The method of claim 8, wherein the first period is at least about 1, 2, or 3 days, the second period is at least about 1, 2, or 3 days, and the third period is at least about 1, 2, 3, 4, or 5 days; and/or wherein the fourth period is at least about 3, 4, 5, 6, 7, or 8 days, and the fifth period is at least about 1, 2, 3, or 4 days.

10. (canceled)

11. (canceled)

12. The method of claim 1, wherein the gut endoderm spheroids are contacted with the SM at a ratio of about 250, about 500, about 1000, about 1500, about 2000, about 2500, about 3000, about 3500, about 4000, about 4500, or about 5000 SM per gut endoderm spheroid, or any ratio within a range defined by any two of the aforementioned ratios of SM to gut endoderm spheroid, or wherein the gut endoderm spheroids are contacted with the SM at a ratio of about 1 to 1, 1.5 to 1, 2 to 1, 2.5 to 1, or 3 to 1 SM to the total number of gut endoderm cells in the gut endoderm spheroid, or any ratio within a range defined by any two of the aforementioned ratios of SM to the total number of gut endoderm cells in the gut endoderm spheroid; and/or
wherein the gut endoderm spheroids are contacted with the ENCCs at a ratio of about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 ENCCs per gut endoderm spheroid, or any ratio within a range defined by any two of the aforementioned ratios of ENCCs to gut endoderm spheroid, or wherein the gut endoderm spheroids are contacted with the ENCCs at a ratio of about 1 to 1, 1 to 1.25, 1 to 1.5, 1 to 2, 1 to 2.5, or 1 to 3 ENCCs to the total number of gut endoderm cells in the gut endoderm spheroid, or any ratio within a range defined by any two of the aforementioned ratios of ENCCs to the total number of gut endoderm cells in the gut endoderm spheroid.

13. (canceled)

14. (canceled)

15. (canceled)

16. The method of claim 1, wherein the SM or ENCCs, or both, are in suspension of single cells; and/or wherein the gut endoderm spheroids are contacted with the SM and the ENCCs by low speed centrifugation; and/or wherein the cell mixture is cultured in an extracellular matrix or a derivative or mimic thereof.

17. (canceled)

18. (canceled)

19. The method of claim 1, wherein the gut endoderm spheroids are foregut endoderm spheroids.

20. The method of claim 19, wherein the foregut endoderm spheroids are posterior foregut endoderm spheroids, and the gastrointestinal organoid is a gastric organoid.

21. The method of claim 20, wherein the posterior foregut endoderm spheroids have been derived from definitive endoderm cells according to a method comprising activating an FGF pathway, a BMP pathway inhibitor, and a Wnt pathway in the definitive endoderm cells for a first period and activating an FGF pathway, a BMP pathway inhibitor, and a Wnt pathway, in combination with retinoic acid, for a second period, thereby differentiating the definitive endoderm cells into the posterior foregut endoderm spheroids; and/or
wherein the gastric organoid is an antral gastric organoid and the conditions sufficient to differentiate the cell mixture to the antral gastric organoid comprises inhibiting a BMP pathway and activating an EGF pathway, in combination with retinoic acid, in the cell mixture for a third period, and activating an EGF pathway activator for a fourth period; and/or
wherein the gastric organoid is a fundic gastric organoid and the conditions sufficient to differentiate the cell mixture to the fundic gastric organoid comprises inhibiting a BMP pathway and activating Wnt pathway and an EGF pathway, in combination with retinoic acid, in the cell mixture for a fifth period, and activating a Wnt pathway and an EGF pathway for a sixth period, and/or wherein, in the fundic gastric organoid, a BMP pathway is further activated and a MEK pathway is inhibited, to induce parietal cell differentiation.

22. The method of claim 21, wherein the first period is 1, 2, 3, 4, or 5 days, preferably 3 days, and the second period is 1, 2, or 3 days; and/or wherein the third period is 1, 2, 3, 4, or 5 days, and the fourth period is 1-16 days; and/or wherein the fifth period is 1, 2, 3, 4, or 5 days, and the sixth period is 1-16 days.

23. (canceled)

24. (canceled)

25. The method of claim 23, wherein the antral gastric organoid comprises PDX1 expression, surface mucous cells expressing MUC5AC, gland mucous cells expressing MUC6, or endocrine cells expressing ghrelin, serotonin, histamine, and gastrin, or any combination thereof; and/or wherein the antral gastric organoid comprises a neural plexus comprising choline acetyltransferase+ (CHAT+) and dopaminergic (TH+) neurons in close proximity to the epithelium and/or endocrine cells.

26. (canceled)

27. (canceled)

28. (canceled)

29. (canceled)

30. The method of claim 21, wherein the fundic gastric organoid comprises ATP4B+GIF+ parietal cells, PGA3 expression, and lacks PDX1 and gastrin.

31. The method of claim 20, wherein the gastric organoid comprises about 50% or at least 50% mesenchyme; and/or
wherein the mesenchyme of the gastric organoid is capable of differentiating into αSMA+ smooth muscle cell; and/or
wherein the gastric organoid comprises the gastric epithelial marker CLDN18 and lacks the intestinal epithelial marker CDH17; and/or
wherein the gastric organoid exhibits spontaneous contractile oscillations.

32. (canceled)

33. (canceled)

34. (canceled)

35. The method of claim 19, wherein the foregut endoderm spheroids are anterior foregut endoderm spheroids, and the gastrointestinal organoid is an esophageal organoid.

36. The method of claim 35, wherein the anterior foregut endoderm spheroids have been derived from definitive endoderm cells according to a method comprising activating an FGF pathway and inhibiting a BMP pathway in the definitive endoderm cells for a first period, thereby differentiating the definitive endoderm cells into the anterior foregut endoderm spheroids; and/or
wherein the conditions sufficient to differentiate the cell mixture to the esophageal organoid comprises activating an FGF pathway and an EGF pathway and inhibiting a BMP pathway for a second period, and activating an EGF pathway for a third period.

37. The method of claim 36, wherein the first period is 1, 2, 3, 4, or 5 days; and/or wherein the second period is 1, 2, 3, 4, 5, 6, 7, 8, 9, 10, 11, 12, 13, or 14 days, and the third period is 3-58 days.

38. (canceled)

39. (canceled)

40. The method of claim 35, wherein the esophageal organoid comprises about 25% or at least 25% mesenchymal cells.

41. The method of claim 35, wherein the esophageal organoid comprises about 4% or at least 4% mesenchymal cells expressing FOXF1, and/or wherein the esophageal organoid comprises a TUJ1+ neuronal plexus associated within a FOXF1+ mesenchymal layer.

42. (canceled)

43. (canceled)

44. (canceled)

45. The method of claim 1, further comprising transplanting the gastrointestinal organoid into a mammal, such as a mouse, such as an immunocompromised mouse.

46. (canceled)

47. The method of claim 45, wherein the transplanted gastrointestinal organoid grows about 50×, 150×, 200×, 250×, 300×, 400×, 500×, 600×, 700×, 800×, 900×, 1000×, 1100×, 1200×, 1300×, 1400×, or 1500×, or at least 50×, 150×, 200×, 250×, 300×, 400×, 500×, 600×, 700×, 800×, 900×, 1000×, 1100×, 1200×, 1300×, 1400×, or 1500× in volume following transplantation and/or comprises αSMA+ smooth muscle cells, enteric neurons and epithelium.

48. (canceled)

49. (canceled)

50. (canceled)

51. A gastrointestinal organoid produced by the method of claim 1.

52. The gastrointestinal organoid of claim 51, wherein the gastrointestinal organoid comprises a muscularis mucosa, submucosa, and muscularis externa, and/or wherein the gastrointestinal organoid comprises plexi of enteric neurons within the submucosa and muscularis externa.

53.-75. (canceled)

76. A method of screening, comprising contacting the gastrointestinal organoid of claim 51 with a compound of interest and assessing a change in phenotype in the gastrointestinal organoid.

77. The method of claim 76, wherein the gastrointestinal organoid is derived from stem cells obtained from a subject, and wherein the subject comprises a disease and the change in phenotype is associated with an improvement of the disease.

78. (canceled)
